SEQUENCE LISTING (61 sequences)

SEQ ID NO: 1 - protein, 243 aa, Escherichia coli
MSTAITRQIVLDTETTGMNQIGAHYEGHKIIEIGAVEVVNRRLTGNNFHVYLKPDRLVDPEAFGVHGIADEFLLDKPTFAEVADEFMDYIRGAELVIHNAAFDIGFMDYEFSLLKRDIPKTNTFCKVTDSLAVARKMFPGKRNSLDALCARYEIDNSKRTLHGALLDAQILAEVYLAMTGGQTSMAFAMEGETQQQQGEATIQRIVRQASKLRVVFATDEEIAAHEARLDLVQKKGGSCLWRA

SEQ ID NO: 2 - DNA, 750 bp, Escherichia coli
cacaggtatttatgctcgccagaggcaacttccgcctttcttctgcaccagatcgagacgggcttcatgagctgcaatctcttcatctgtcgcaaaaacaacgcgtaacttacttgcctgacgtacaatgcgctgaattgttgcttcaccttgttgctgttgtgtctctccttccatcgcaaaagccatcgacgtttgaccaccggtcatcgccagataaacttccgcaaggatctgggcatcgagtaatgccccgtgcagcgttcgtttactgttatctatttcgtagcgagcacataacgcatcgaggctgttgcgcttaccgggaaacattttcctcgccaccgcaaggctatcggtgaccttacagaaagtattggtcttcggaatatcgcgcttaagcaacgaaaactcgtagtccataaagccgatatcgaacgctgcgttatggatcaccaactccgcgccgcgaatatagtccatgaactcatcggctacttcggcaaacgtgggcttatcgagcaaaaattcatcggcaataccatgtacgccaaaggcttccggatccaccagccgatcgggtttgagataaacatggaagttattgcccgtcaggcgacggttcaccacttcaacggcaccaatctcaatgatcttgtggccttcatagtgcgcaccaatctggttcataccggtggtttcggtatcgagaacgatctggcgtgtaattgcagtgctcatagcggtcat

SEQ ID NO: 3 - protein, 243 aa, Escherichia coli; identical to SEQ ID NO: 1

SEQ ID NO: 4 - protein, 243 aa, Escherichia coli; identical to SEQ ID NO: 1

SEQ ID NO: 5 - protein, 243 aa, Shigella flexneri
MSTAITRQIVLDTETTGMNQIGAHYEGHKIIEIGAVEVVNRRLTGNNFHVYLKPDRLVDPEAFGVHGIADEFLLDKPTFAEVADEFMDYIRGAELVIHNAAFDIGFMDYEFSLLKRDIPKTNTFCKVTDSLAVARKMFPGKRNSLDALCARYEIDNSKRTLHGALLDAQILAEVYLAMTGGQTSMAFAMEGETQQQQGEATIQRIVRQASKLRVVFATDEELAAHEARLDLVQKKGGSCLWRA

SEQ ID NO: 6 - protein, 243 aa, Shigella flexneri; identical to SEQ ID NO: 5

SEQ ID NO: 7 - protein, 243 aa, Escherichia coli
MSTAITRQIVLDTETTGMNQIGAHYEGHKIIEIGAVEVVNRRLTGNNFHVYLKPDRLVDPEAFGVHGIADEFLLDKPTFAEVADEFMDYIRGAELVIHNAAFDIGFMDYEFSLLKRDIPKTNTFCKVTDSLAVARKMFPGKRNSLDALCARYEIDNSKRTLHGALLDAQILAEVYLAMTGGQTSMAFAMEGETQQQQGEATIQRLVRQASKLRVVFATDEELAAHEARLDLVQKKGGSCLWRA

SEQ ID NO: 8 - protein, 243 aa, Escherichia coli
MSTAITRQIVLDTETTGMNQIGAHYEGHKIIEIGAVEVVNRRLTGNNFHVYLKPDRLVDPEAFGVHGIADEFLLDKPTFAEVADEFMDYIRGAELVIHNAAFDIGFMDYEFSLLKRDIPKTNTFCKVTDSLAVARKMFPGKRNSLDALCARYEIDNSKRTLHGALLDAQILAEVYLAMTGGQTSMAFAMEGETQQQQGETTIQRIVRQASKLRVVFATDEELAAHEARLDLVQKKGGSCLWRA

SEQ ID NO: 9 - protein, 243 aa, Salmonella enterica
MSTAITRQIVLDTETTGMNQIGAHYEGHKIIEIGAVEVINRRLTGNNFHVYLKPDRLVDPEAFGVHGIADEFLLDKPVFADVVDEFLDYIRGAELVIHNASFDIGFMDYEFGLLKRDIPKTNTFCKVTDSLALARKMFPGKRNSLDALCSRYEIDNSKRTLHGALLDAQILAEVYLAMTGGQTSMTFAMEGETQRQQGEATIQRIVRQASRLRVVFASEEELAAHESRLDLVQKKGGSCLWRA

SEQ ID NO: 10 - protein, 242 aa, Photorhabdus luminescens
MSTAITRQVVLDTETTGMNKLGVHYEGHKIIEIGAVEVVNRRLTGRHFHVYIQPDRLVDPEAFEVHGISDEFLQDKPLFADVADEFVEFIRGAELIIHNAPFDIGFIDYEFGKLDRDIPPTADFCKITDSLQLARGLFPGKRNNLDALCDRYDIDNSKRTLHGALLDAEILADVYLIMTGGQTSLAFSMEGEIAGGANVSEIQRVTRSQTALKVVYATDEELAAHESRLDLVEKKGGSCLWR

SEQ ID NO: 11 - protein, 237 aa, Yersinia pestis
TRQIVLDTETTGMNKLGVHYEGHRIIEIGAVEVINRRLTGRNFHVYVKPDRLVDPEAYGVHGISDEFLADKPTFADITPEFLDFIRGAELVIHNAAFDIGFMDYEFRMLQQDIPKTETFCTITDSLLMARRLFPGKRNNLDALCDRYQIDNTKRTLHGALLDAEILAEVYLAMTGGQTSLTFSMEGEVSQNNASEDIQRITRPASALKIIYATEDELANHESRLDFVMKKGGSCLWR

SEQ ID NO: 12 - protein, 242 aa, Photorhabdus luminescens
MSTAITRQVVLDTETTGMNKLGVHYEGHKIIEIGAVEVINRRLTGRHFHVYIQPDRLVDPEAFEVHGISDEFLQDKPLFADIADEFIEFIRGAELIIHNAPFDIGFIDYEFGKLNRDIPPTADFCKITDSLQLARGLFPGKRNNLDALCDRYDIDNSKRTLHGALLDAEILSDVYLIMTGGQTSLAFSMEGEIASGANVSEIQRITRPQMALKVIYATDEELAAHESRLDLVEKKGGSCLWR

SEQ ID NO: 13 - protein, 186 aa, Escherichia coli
MSTAITRQIVLDTETTGMNQIGAHYEGHKIIEIGAVEVVNRRLTGNNFHVYLKPDRLVDPEAFGVHGIADEFLLDKPTFAEVADEFMDYIRGAELVIHNAAFDIGFMDYEFSLLKRDIPKTNTFCKVTDSLAVARKMFPGKRNSLDALCARYEIDNSKRTLHGALLDAQILAEVYLAMTGGQTSMA

SEQ ID NO: 14 - protein, 238 aa, Anopheles gambiae
LNRQIILDTETTGMNTAGGPVYLGHRIIEIGCVEVINRKLTGNHFHVYIKPDRLVDPEAIQVHGITDEFLRDKPSFSQIADEFIEFIRGAELIAHNAPFDVSFMDYEFGKLGLNFKTADICGITDTLAMARDLFPGKRNNLDVLCDRYGIDNSHRTLHGALLDAEILADVYLLMTGGQTKLNLATESSENESNQDTSIRRLESNRPPLKVIRASAEIEAVHEARLDLVQKKGGACLWR

SEQ ID NO: 15 - protein, 236 aa, Pasteurella multocida
TRQIVLDTETTGMNQFGAHYEGHCIIEIGAVEMINRRLTGNNFHIYIKPNRPVDPDAIKVHGITDEMLADKPMFNEVAQQFIDYIQGAELLIHNAPFDVGFMDYEFKKLNLNINTDAICMVTDTLQMARQMYPGKRNSLDALCDRLGIDNSKRTLHGALLDAEILADVYLTMTGGQTSLFDENEPEIAVVAVQEQIQSAVAFSQDLKRLQPNADELQAHLDYLLLLNKKSKGNCLW

SEQ ID NO: 16 - protein, 237 aa, Vibrio cholerae
RIVVLDTETTGMNREGGPHYEGHRIIEIGAVEIINRKLTGRHFHVYLKPDRDIQLEAIEVHGITDEFLKDKPEYKDVHEEFVDFIKGAELVAHNAPFDVGFMDYEFAKLGGAIGKTSDFCKVTDTLAMAKRIFPGKRNNLDILCERYGIDNSHRTLHGALLDAEILADVYLLMTGGQTSLQFSSVTQNSGELSAESLKRARSERKALKVLAASADELQAHQDRLDIVAKSGTCLWRS

SEQ ID NO: 17 - protein, 240 aa, Haemophilus influenzae
RQIVLDTETTGMSQLGAHYEGHCIIEIGAVELINRRYTGNNFHIYIKPDRPVDPDAIKVHGITDEMLADKPEFKDVAQDFLDYINGAELLIHNAPFDVGFMDYEFRKLNLNVKTDDICLVTDTLQMARQMYPGKRNNLDALCDRLGIDNSKRTLHGALLDAEILADVYLMMTGGQTNLFDEESVESEVIRVVQEKTAEEIKSAVDFSHNLKLIQPTNDELQAHLEFLKMMNKKSGNNCLW

SEQ ID NO: 18 - protein, 232 aa, Vibrio vulnificus
RIVVLDTETTGMNREGGPHYMGHRIIEIGAVEIINRKLTGRHFHVYLKPDREIQPDAIDVHGITDQFLVDKPEYRQVHQEFLEFIKGAELVAHNAPFDVGFMDYEFGKLDAAIGKTDDYCKVTDTLAMAKKIFPGKRNNLDVLCERYGIDNSHRTLHGALLDAEILADVYLLMTGGQTSLEFNANSQEGGGEDIRRVAGRKSLKVLRATADELEAHQSRLDIVEKSGTCLWR

SEQ ID NO: 19 - protein, 241 aa, Haemophilus influenzae
  misc_feature (42)..(42): Xaa can be any naturally occurring amino acid
RQIVLDTETTGMNQLGAHYEGHCIIEIGAVELINRRYTGNNXHIYIKPDRPXDPDAIKVHGITDEMLADKPEFKEVAQDFLDYINGAELLIHNAPFDVGFMDYEFRKLNLNVKTDDICLVTDTLQMARQMYPGKRNNLDALCDRLGIDNSKRTLHGALLDAEILADVYLMMTGGQTNLFDEEESVESGVIRVMQEKTAEEIKSAVDFSHNLKLLQPTNDELQAHLEFLKMMNKKSGNNCLW

SEQ ID NO: 20 - protein, 242 aa, Haemophilus somnus
MTLEITQNRQIILDTETTGMNEFGAHYEGHCIIEIGAVEMINRRYTGRKLHLYIKPDRLVDPEAIKVHGITDEMLADKPDFSAIAQEFIDFIKGAELIIHNAPFDIGFMDYEFKKHNFNINTADICLITDTLQMARQMYPGKRNSLDALCDRLNIDNSKRTLHGALLDAEILGDVYLAMTGGQTSLFGDEEHTPIITLEENIHQHTTNTHNFKLLLPTEEEKLAHQDYLKLLNQKSKENCLW

SEQ ID NO: 21 - protein, 228 aa, Vibrio parahaemolyticus
RIVVLDTETTGMNQEGGPHYLGHRIIEIGAVEIINRKLTGRHFHVYIKPDREIQPEAIQVHGITDEFLVDKPEYASIHQEFLDFIKGAELVAHNAPFDTGFMDYEFEKLDPTIGKTDDYCKVTDTLAMAKKIFPGKRNNLDVLCERYGIDNSHRTLHGALLDAEILADVYLLMTGGQTSLEFNANKQEGGVETIRRIEGRKALKVLRATADELEAHQKRLELVNDCIW

SEQ ID NO: 22 - protein, 238 aa, Actinobacillus pleuropneumoniae
IVRQVVLDTETTGMSFSGPPQIGHNIIEIGAVEVINRRLTGRTFHVYIKPPREVDEEAIKVHGITNEFLQDKPVFAEVADEFIEFIKGAELIIHNAPFDVAFMDQEFSYLGNPPPKTAEMCSVTDSLAVARKMYPGKRNNLDALCDRLGIDNSKRVLHGALLDAEILADVFLMMTGGQLALLGEEDATATHENVADLGLGTITKFETSGLIVLSLSEEEQTAHEEYLKLIDKKSKGNC

SEQ ID NO: 23 - protein, 255 aa, Candidatus Blochmannia
MNINSNRYVVLDTETTGMNKFGVHYEGHRIIEIGAVEIINRRLTNNQFHVYLNPNRSVDSEAFAIHGISDQFLVDQPCFLDIANDFLQFIRGSTLVIHNASFDLGFLNFELHNIYLNSRTVESYCTVIDSLKLARKIFPGQRNSLDALCERYCIKNSKRILHNALIDAQLLAYVFLVMTGGQTRIQFMDMLDNSDTNILNNTITHDNKFELCLNNSVCTEKKSLKILYATSIEKLEHEKYLDFVMKASNNQCLWR

SEQ ID NO: 24 - protein, 235 aa, Shewanella sp.
SRQVILDTETTGMNQGSGAVYLGHRIIEIGCVEVINRRLTGRYYHQYINPGQAIDPEAIAVHGITDERVANEPRFHQIAQEFIDFISGAEIVAHNANFDVSFMDHEFSLLQPLGPKTRDICEILDSLDIAKFLHPGQKNNLDALCKRYGIDNSRRHYHGALLDAEILADVYLSMTGGQTKFNLSNEEVGQEGGGIQRFDPTSLNLKVICASADELVMHEKRLDLVAKSGKCLWRG

SEQ ID NO: 25 - protein, 239 aa, Haemophilus ducreyi
IIRQVVLDTETTGMNFNGPPQIGHNIIEIGAVELINRRLTGRTFHVYIKPPREVEEEAIKVHGITNAFLQDKPTFAEIAHEFLAFIQGAELIIHNAPFDVAFIDQEFSSLVNPPSKTAEMCTVTDTLQMARKMYPGKRNNLDALCDRLGIDNSKRVLHGALLDAEILADVFLMMTGGQLALLTEEEHSHTQQQRETSLAVKEHFDTSGLIVLQLSQEECQAHQEYLALLDKKSKGNCLW

SEQ ID NO: 26 - protein, 231 aa, Burkholderia sp.
RQLILDTETTGLNARTGDRILELGCVELVNRRLTGNNLHFYINPERDSDPGALAVHGLTTEFLSDKPKFGEIADQFRDFIQGADLIIHNAPFDIGFLDVEFALLGLPPVSTYCGEIIDTLARAKQMFPGKRNSLDALCDRFGISNAHRTLHGALLDSELLAEVYLAMTRGQESLVIDMLGESHAGGDARAPRVAIDSLDLVVITASDDELAAHQALLDGLDKAIKGTSVWR

SEQ ID NO: 27 - protein, 188 aa, Wigglesworthia glossinidia
MKINTERQIVLDTETTGMNKNGPHYYGHRIIEIGAIEMINRRLTGRCFHTYLKPDRLVEIEAFKIHGISDEFLFFQPTFEEIMEKFINFIKGSELIIHNSVFDIGFINNEIQLCNKNLNNINYYCSVIDTLKLARNIFPGKRNNLDALSDRYGIDTTKRILHGALLDAEILSNVYLLMTGGQIPINFS

SEQ ID NO: 28 - protein, 236 aa, Pseudomonas aeruginosa
RSVVLDTETTGMPVTDGHRIIEIGCVELEGRRLTGRHFHVYLQPDREVDEGAIAVHGITNEYLKDKPRFREVANDFFEFIRGAQLIIHNAAFDIGFINNEFALLGQQDRSDVSEYCSVLDTLLMARERHPGQRNNLDALCKRYGVDNSGRDLHGALLDAEILADVYLAMTGGQTSLSLAGSGAEGDGSGRPMVSPIRRLDPARVATPVLRANAEELAAHAARLAVIEKSAGGPSLW

SEQ ID NO: 29 - protein, 230 aa, Azotobacter vinelandii
RSVVLDTETTGMPVTEGHRIIEIGCVELQGRRLTGRHFHVYLQPDRTVDEGAVAVHGITDDFLADKPRFADIAEEFFEFIKGAQLIIHNAAFDIGFIEDEFSRLGQTERADVNAHCTVLDTLLMARERHPGQRNSLDALCKRYDVDNSNRDLHGALLDAEILADVWLAMTGGQTHLSLSGEGSENGGRAQASAIRRLSPERQRTRVIRAGEQELAAHAERLAAIEKAAGA

SEQ ID NO: 30 - protein, 236 aa, Pseudomonas aeruginosa
RSVVLDTETTGMPVTDGHRIIEIGCVELEGRRLTGRHFHVYLQPDREVDEGAIAVHGITNEYLKDKPRFREVANDFFEFIRGAQLIIHNAAFDIGFINNEFALLGQQDRSDVTEYCSVLDTLLMARERHPGQRNNLDALCKRYGVDNSGRDLHGALLDAEILADVYLAMTGGQTSLSLAGSGAEGDGSGRPMVSPIRRLDPARVATPVLRANAEELAAHAARLAVIEKSAGGPSLW

SEQ ID NO: 31 - protein, 233 aa, Microbulbifer degradans
RQIVLDTETTGLEPSQGHRIIEIGCVELINRKLTGRHYHQYIKPEREIDEGAIEVHGITNEFLADKPVFKDIADEFMAFVDGAELVIHNAPFDVGFLNHEFNLLGRGSTVINDRCSILDTLALARNKHPGQKNNLDALCKRYGADNSARDLHGALLDAEILADVYLLMTGGQTNLALGGAGSSSGMDDGGEELVRVSADRKPLPIIRASAEELALHEKKLAEIDKASGGECLW

SEQ ID NO: 32 - protein, 237 aa, Pseudomonas putida
FVILDTETTGMPVGEGHRIIEIGCVEVIGRRLTGRHFHVYLQPDRESDEGAINVHGITDAFLVGKPRFGDVAEEFFQFIQGATLVIHNAAFDVGFINNEFALLGQQDRADISQHCTILDTLLLARSRHPGQRNSLDALCKRYDIDNSGRELHGALLDSELLADVYLAMTGGQTSLSLAGNGADTEEDGQGAGGSEIRRIVGRAPGRVIMASAEELEAHAERLAAIAKSAGGPSLWQA

SEQ ID NO: 33 - protein, 245 aa, Pseudomonas syringae
MQNLDNRSIVLDTETTGMPVTDGHRIVEIGCVELIGRRLTGRHFHVYLQPDRESDEGAIGVHGITNEFLVGKPRFAEVADEFFEFIKGAQLIIHNAAFDVGFINNEFALMGAQDKADITRHCKILDTLMMARERHPGQRNSLDALCKRYGVDNSGRELHGALLDSEILADVYLAMTGGQTSLSLAGNASDGNGSAEGSGNRGSEIRRLPADRKPCRVIRASESELAEHEVRMTTIAKATGAPALW

SEQ ID NO: 34 - protein, 240 aa, Pseudomonas fluorescens
TRSVVLDTETTGMPVTDGHRIIEIGCVELIGRRLTGRHFHVYLQPDRESDEGAIAVHGITNEFLVGKPRFAEVADEFFEFINGAQLIIHNAAFDVGFINNEFALMGQHDRADITQHCTILDTLMMARERHPGQRNSLDALCKRYGVDNSGRELHGALLDSEILADVYLTMTGGQTSLSLAGNASDGNGTGEGADNSATEIRRLPADRQPGRIIRATEAELAEHQVRLEIIAKSAGAPALW

SEQ ID NO: 35 - protein, 239 aa, Pseudomonas syringae
RSIVLDTETTGMPVTDGHRIIEIGCVELIGRRLTGRHFHVYLQPDRESDEGAIGVHGITNEFLVGKPRFAEVADEFFEFIKGAQLIIHNAAFDVGFINNEFALMGSQDRADITQHCSILDTLMMARERHPGQRNSLDALCKRYGVDNSGRELHGALLDSEILADVYLAMTGGQTSLSLAGNASDGNGSGEGSGNRGSEIRRLPADRKPCRIIRASESELAEHEVRMSTIAKACGAPPLW

SEQ ID NO: 36 - protein, 234 aa, Buchnera aphidicola
RKIVLDIETTGMNPAGCFYKNHKIIEIGAVEMINNVFTGNNFHSYIQPNRLIDKQSFKIHGITDNFLLDKPKFHEISVKFLEYITNSDLIIHNAKFDVGFINYELNMINSDKRKISDYCNVVDTLPLARQLFPGKKNSLDALCNRYKINVSHRDFHSALIDAKLLAKVYTFMTSFQQSISIFDKNSNLNSIQKNAKLDSRVPFRSTLLLATKDELQQHMKYLKYVKQETGNCVW

SEQ ID NO: 37 - protein, 231 aa, Bordetella pertussis
RQIIFDTETTGLDPAQGHRIVEIGCVEIVNRMVTGNNLHLYLNPDRDSDPEALAVHGLTTEFLADKPRFAEVAEQFLAFIADAELIAHNAAFDVKFFNAELQRIGRDPLNTHCENIVDSLLHARSLHPGKRNSLDALCDRYGISNAHRTLHGALLDSQLLAEVWLAMTRGQDALLIDVDDQGANANGALVLGKFDASVLTVLAASEAELAEHAAYLQALDKAVGGACAWRA

SEQ ID NO: 38 - protein, 232 aa, Buchnera aphidicola
RTIILDTETTGINQTSLPHINHRIIEIGAVEIIDRCFTGNNFHVYIQPGRSIESGALKVHGITNKFLLDKPIFKDIADSFLNYIKNSILVIHNASFDVGFINQELEILNKKIKINTFCSIIDTLKIARELFPGKKNTLDALCTRYKINKSHRNLHSAIVDSYLLGKLYLLMTGGQDSLFSDNTINYKENFKKLKKNIQLKNNTLRILHPTLKENDLHEKYLQYMKDKSTCLW

SEQ ID NO: 39 - protein, 228 aa, Coxiella burnetii
RQIVLDTETTGLVPEEGHRIIEIGALEMVNRRLTGNHLHFYINPERSIERDAIEIHGITDSFLIDKPLFKDIATELISFLKGAELIIHNAPFDVGFLNHELKLTGQSFKTLTHYCQVLDTLTIARQKHPGQHNNLDALCRRYHVDNSNRDYHGALLDAELLAQVYLLMTGGQTVLFEQQGFAVASRSVSVRPLGTDRDSLSVIRANAAETEAHRAFLQLLTENGLCLW

SEQ ID NO: 40 - protein, 234 aa, Xanthomonas campestris
RQIILDTETTGLEWRKGNRVVEIGAVELLERRPSGNNFHRYLRPDCDFEPGAQEVTGLTLEFLADKPVFAEVVEEFLAYIDGAELIIHNAAFDLGFLDNELSLLGDQFGRIIDRATVVDTLMMARERYPGQRNSLDALCKRLGVDNSHRQLHGALLDAQILADVYIALTSGQEEIGFGAMDAGQHAEGGEGMIAFDPSLLLPRPRVVVTPSELQAHEARLERLRKKAGRALWDA

SEQ ID NO: 41 - protein, 234 aa, Buchnera aphidicola
MMNNTQRIIVLDTETTGMNSVGPPYLNHRIIEIGAIEIINRRFTGKKFHTYIKPNRLIESDASKIHGITDDFLSDKPSFKDIAKDFFNYIKNSELIIHNASFDVGFINQEFSMLTKKIQDISNFCNIIDTLKIARKLFPGKKNTLDALCMRYKIKNSHRVLHGAILDAFLLGKLYLLMTSGQESIIFNKNIQNERNFRYIKKSITKKHRFLKIIKANKTELKLHNEYLKFLKEK

SEQ ID NO: 42 - protein, 228 aa, Buchnera aphidicola
RIIVLDTETTGMNSVGPPYLNHRIIEIGAIEIINRRFTGKKFHTYIKPNRLIESDASKIHGITDDFLSDKPSFKDIAKDFFNYIKNSELIIHNASFDVGFINQEFSMLTKKIQDISNFCNIIDTLKIARKLFPGKKNTLDALCMRYKIKNSHRVLHGAILDAFLLGKLYLLMTSGQESIIFNKNIQNERNFRYIKKSITKKHRFLKIIKANKTELKLHNEYLKFLKEK

SEQ ID NO: 43 - protein, 233 aa, Xylella fastidiosa
RQIVLDTETTGLEWSKGNRIVEIGAVELLDRRLSGDKFHRYLKPDVSFESGAQEVTGLTMEFLADKPEFSMIADEFLAYINGAELIIHNAAFDLGFLDYELSRLGSQYGKITDRASVLDTLVMARERYPGQRNSLDALCKRLGVDNAHRQLHGALLDAQILADVYIALTSGQEEIGFALPESSRGGVDAASVAFMPDVLLTRPCVVVSQSELEAHEARLAKLRKIAGHVLWDA

SEQ ID NO: 44 - protein, 233 aa, Xylella fastidiosa
RQIVLDTETTGLEWSKGNRIVEIGAVELLDRRLSGDKFHRYLKPDVSFESGAQEVTGLTMEFLADKPEFSMIADKFLAYINGAELIIHNAAFDLGFLDYELSRLGSQYGKITDRASVLDTLVMARERYPGQRNSLDALCKRLGVDNAHRQLHGALLDAQILADVYIALTSGQEEIGFALPESSRGGVDAASVAFMPDVLLTRPCVVASQSELEAHEARLAKLRKIAGHVLWDA

SEQ ID NO: 45 - protein, 233 aa, Xylella fastidiosa
RQIVLDTETTGLEWSKGNRIVEIGAVELLDRRLSGDKFHRYLKPDVSFESGAQEVTGLTMEFLADKPEFSMIADEFLAYINGAELIIHNAAFDLGFLDYELSRLGSQYGKITDRASVLDTLVMARERYPGQRNSLDALCKRLGVDNAHRQLHGALLDAQILADVYIALTCGQEEIGFALPESSCGGVDAASAAFMPDVLLTRPCVVVSQSELEAHEARLAKLRKIAGHVLWDA

SEQ ID NO: 46 - protein, 229 aa, Chromobacterium violaceum
RQIILDTETTGLDPQQGHRIIEFAGLEMVGRKLTGKHLHLYIHPEREIDPEAQRVHGISLEFLAGKPVFAKVAHEIADFLRDAELIIHNAPFDVGFLNAEFAKAGIEPVGKLCASVIDTLAEARDMFPGKRNSLDALCDRFEIDRSNRTLHGALVDCELLSEVYLWMTRGQESLAMDIEVELPGGDAGAIQFERKPLKVLAASEAEEAEHQAYLDVLDKAVKGICVWRG

SEQ ID NO: 47 - protein, 132 aa, Salmonella typhimurium
  misc_feature (90)..(91): Xaa can be any naturally occurring amino acid
MSTAITRQIVLDTETTGMNQIGAHYEGHKIIEIGAVEVINRRLTGNNFHVYLKPDRLVDPEAFGVHGIADEFLLDKPVFADVVDEFLDYXXGAELVIHNAXXXXXXXXXXXXXXXXXXPKTNTFCKVTDSLA

SEQ ID NO: 48 - protein, 230 aa, Ralstonia sp.
RQIVLDTVTTGLNHATGDRLIEIGCVELVNRRLTGRHLHFYVNPERDIHEDAIAVHGITLDFLADKPKFAEIVNDVRDFVQDAELIIHNAPFDLGFLDMEFQRLDLPPFRQHASNVIDTLREARQMFPGKRNSLDALCDRLGVSNSHRTLHGALLDAELLAEVYLAMTRGQNSLVIDMLDGAATDGETRSTADLSAMTLPVLLASEAEISAHMGVLKELDKASGGKTVWQ

SEQ ID NO: 49 - protein, 232 aa, Xanthomonas axonopodis
RQIILDTETTGLEWRKGNRVVEIGAVELLERRPSGNNFHRYLKPDCDFEPGAQEVTGLTLEFLADKPLFGEVVDEFLAYIDGAELIIHNAAFDLGFLDNELALLGDHYGRIVERATVVDTLMMARERYPGQRNSLDALCKRLGVDNSHRQLHGALLDAQILADVYIALTSGQEEIGFASADAGQQADAASGMIAFDPALLLPRPRVAVTASESQAHEARLAQLRKKAGRALW

SEQ ID NO: 50 - protein, 173 aa, Buchnera aphidicola
RIIVLDTETTGMNSVGPPYLNHRIIEIGAIEIINRRFTGKKFHTYIKPNRLIESDASKIHGITDDFLSDKPSFKDIAKDFFNYIKNSELIIHNASFDVGFINQEFSMLTKKIQDISNFCNIIDTLKIARKLFPGKKNTLDALCMRYKIKNSHRVLHGAILDAFLLGKLYLLMT

SEQ ID NO: 51 - protein, 184 aa, Ralstonia solanacearum
RQIVLDTETTGLNAATGDRVIEIGCVELVNRRLTGRNLHFYLNPEREIDAGAMAVHGITNEFVADKPKFAEVVDEIRDYVQGAEAIIHNAAFDLGFLDMEFKRLGLPPFREHLAGHIDTLLDARRMFPGKRNSLDALCDRLGVSNAHRTLHGALLDAELLAEVYLAMTRGQNTLVIDMLESGET

SEQ ID NO: 52 - protein, 221 aa, Rickettsia prowazekii
REIILDTETTGLDPQQGHRIVEIGAIEMVNKVLTGKHFHFYINPERDMPFEAYKIHGISGEFLKDKPLFKTIANDFLKFIADSTLIIHNAPFDIKFLNHELSLLKRTEIKFLELTNTIDTLVMARNMFPGARYSLDALCKRFKVDNSGRQLHGALKDAALLAEVYVALTGGRQSTFKMINKPDEINNLAVKCVDVQQIKRGIVVKPTKEELQKHKEFIDKI

SEQ ID NO: 53 - DNA, 147 bp, Artificial
  T7 Promoter
gcattagcggccaaattaatacgactcactatagggccgtcgttttacaacgtcgtgactgggaaaaccctggcgttacccaacttaatcgccttgcagcacatccccctttcgccagctggcgtaatagcgaagaggcccgcaccg

SEQ ID NO: 54 - DNA, 15 bp, Artificial
  Primer
cggtgcgggcctctt

SEQ ID NO: 55 - DNA, 30 bp, Artificial
  Primer
aagaagtagaggactgttatgaaagagaag

SEQ ID NO: 56 - DNA, 21 bp, Artificial
  Primer
catccatgactccgccatctg

SEQ ID NO: 57 - DNA, 20 bp, Artificial
  Primer
aatttgtgcaaagttgagtc

SEQ ID NO: 58 - DNA, 732 bp, Escherichia coli
atgagcactgcaattacacgccagatcgttctcgataccgaaaccaccggtatgaaccagattggtgcgcactatgaaggccacaagatcattgagattggtgccgttgaagtggtgaaccgtcgcctgacgggcaataacttccatgtttatctcaaacccgatcggctggtggatccggaagcctttggcgtacatggtattgccgatgaatttttgctcgataagcccacgtttgccgaagtagccgatgagttcatggactatattcgcggcgcggagttggtgatccataacgcagcgttcgatatcggctttatggactacgagttttcgttgcttaagcgcgatattccgaagaccaatactttctgtaaggtcaccgatagccttgcggtggcgaggaaaatgtttcccggtaagcgcaacagcctcgatgcgttatgtgctcgctacgaaatagataacagtaaacgaacgctgcacggggcattactcgatgcccagatccttgcggaagtttatctggcgatgaccggtggtcaaacgtcgatggcttttgcgatggaaggagagacacaacagcaacaaggtgaagcaacaattcagcgcattgtacgtcaggcaagtaagttacgcgttgtttttgcgacagatgaagagattgcagctcatgaagcccgtctcgatctggtgcagaagaaaggcggaagttgcctctggcgagcataa

SEQ ID NO: 59 - protein, 243 aa, Escherichia coli; identical to SEQ ID NO: 1

SEQ ID NO: 60 - DNA, 570 bp, Thermotoga maritima
gtgctcgccatgatatggaacgacaccgttttttgcgtcgtagacacagaaaccacgggaaccgatccctttgccggagaccggatagttgaaatagccgctgttcctgtcttcaaggggaagatctacagaaacaaagcgtttcactctctcgtgaatcccagaataagaatccctgcgctgattcagaaagttcacggtatcagcaacatggacatcgtggaagcgccagacatggacacagtttacgatcttttcagggattacgtgaagggaacggtgctcgtgtttcacaacgccaacttcgacctcacttttctggatatgatggcaaaggaaacgggaaactttccaataacgaatccctacatcgacacactcgatctttcagaagagatctttggaaggcctcattctctcaaatggctctccgaaagacttggaataaaaaccacgatacggcaccgtgctcttccagatgccctggtgaccgcaagagtttttgtgaagcttgttgaatttcttggtgaaaacagggtcaacgaattcatacgtggaaaacgggggtaa

SEQ ID NO: 61 - protein, 189 aa, Thermotoga maritima
VLAMIWNDTVFCVVDTETTGTDPFAGDRIVEIAAVPVFKGKIYRNKAFHSLVNPRIRIPALIQKVHGISNMDIVEAPDMDTVYDLFRDYVKGTVLVFHNANFDLTFLDMMAKETGNFPITNPYIDTLDLSEEIFGRPHSLKWLSERLGIKTTIRHRALPDALVTARVFVKLVEFLGENRVNEFIRGKRG

(19) United States
(12) Patent Application Publication
(10) Pub. No.: US 2005/0272074 A1
(43) Pub. Date: Dec. 8, 2005

(54) Compositions and methods for reverse transcription

(75) Inventor: Bahram Arezi, Carlsbad, CA (US)

Correspondence Address: PALMER & DODGE, LLP;KATHLEEN M. WILLIAMS / STR, 111 HUNTINGTON AVENUE, BOSTON, MA, 02199, US

(73) Assignee: Stratagene California

(21) Appl. No.: 11/100,183

(22) Filed: Apr. 6, 2005

Related U.S. Application Data
(60) Provisional application No. 60/559,810, filed on Apr. 6, 2004.

(51) Int. Cl. C12Q 1/68; C12N 9/22

(52) U.S. Cl. 435/6; 435/199

(57) ABSTRACT

The present invention provides compositions and methods for high fidelity cDNA synthesis. In particular, the composition of the present invention contains a first enzyme exhibiting a reverse transcriptase activity and a second enzyme comprising a 3′-5′ exonuclease activity.

### RELATED APPLICATION(S)

This application claims the benefit of U.S. Provisional Application No. 60/559,810, filed on Apr. 6, 2004. The entire teachings of the above application(s) are incorporated herein by reference.

### BACKGROUND

One common approach to the study of gene expression is the production of complementary DNA (cDNA). Discovery of an RNA-dependent DNA polymerase, so-called a reverse transcriptase (RT), from a retrovirus has enabled a reverse transcription reaction in which a cDNA is synthesized using an RNA as a template. As a result, methods for analyzing mRNA molecules have made rapid progress. The methods for analyzing MRNA molecules using a reverse transcriptase have now become indispensable experimental methods for studying gene expression and function. Subsequently, these methods, which have been applied to cloning and PCR techniques, have also become indispensable techniques in a wide variety of fields including biology, medicine and agriculture.

Three prototypical forms of retroviral RT have been studied thoroughly. Moloney Murine Leukemia Virus (M-MLV) RT contains a single subunit of 78 kDa with RNA-dependent DNA polymerase and RNase H activity. This enzyme has been cloned and expressed in a fully active form in E. coli (reviewed in Prasad, V. R., Reverse Transcriptase, Cold Spring Harbor, N.Y.: Cold Spring Harbor Laboratory Press, p. 135 (1993)). Human Immunodeficiency Virus (HIV) RT is a heterodimer of p66 and p51 subunits in which the smaller subunit is derived from the larger by proteolytic cleavage. The p66 subunit has both a RNA-dependent DNA polymerase and an RNase H domain, while the p51 subunit has only a DNA polymerase domain. Active HIV p66/p51 RT has been cloned and expressed successfully in a number of expression hosts, including E. coli (reviewed in Le Grice, S. F. J., Reverse Transcriptase, Cold Spring Harbor, N.Y.: Cold Spring Harbor Laboratory press, p. 163 (1993)). Within the HIV p66/p51 heterodimer, the 51-kD subunit is catalytically inactive, and the 66-kD subunit has both DNA polymerase and RNase H activity (Le Grice, S. F. J., et al., EMBO Journal 10:3905 (1991); Hostomsky, Z., et al., J. Virol. 66:3179 (1992)). Avian Sarcoma-Leukosis Virus (ASLV) RT, which includes but is not limited to Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus UR2AV RT, Avian Sarcoma Virus Y73 Helper Virus YAV RT, Rous Associated Virus (RAV) RT, and Myeloblastosis Associated Virus (MAV) RT, is also a heterodimer of two subunits, alpha (approximately 62 kDa) and beta (approximately 94 kDa), in which alpha is derived from beta by proteolytic cleavage (reviewed in Prasad, V. R., Reverse Transcriptase, Cold Spring Harbor, N.Y.: Cold Spring Harbor Laboratory Press (1993), p. 135). ASLV RT can exist in two additional catalytically active structural forms, beta beta and alpha (Hizi, A. and Joklik, W. K., J. Biol. Chem. 252: 2281 (1977)). Sedimentation analysis suggests alpha beta and beta beta are dimers and that the alpha form exists in an equilibrium between monomeric and dimeric forms (Grandgenett, D. P., et al., Proc. Nat. Acad. Sci. USA 70: 230 (1973); Hizi, A. and Joklik, W. K., J. Biol. Chem. 252: 2281 (1977); and Soltis, D. A. and Skalka, A. M., Proc. Nat. Acad. Sci. USA 85: 3372 (1988)). The ASLV alpha beta and beta beta RTs are the only known examples of retroviral RT that include three different activities in the same protein complex: DNA polymerase, RNase H, and DNA endonuclease (integrase) activities (reviewed in Skalka, A. M., Reverse Transcriptase, Cold Spring Harbor, N.Y.: Cold Spring Harbor Laboratory Press (1993), p. 193). The alpha form lacks the integrase domain and activity.

As noted above, the conversion of mRNA into cDNA by RT-mediated reverse transcription is an essential step in many gene expression studies. However, the use of unmodified RT to catalyze reverse transcription is inefficient for at least two reasons. First, RT sometimes renders an RNA template unable to be copied before reverse transcription is initiated or completed, primarily due to the intrinsic RNase H activity present in RT. Second, RTs generally have low fidelity. That is, RTs incorporate mismatched bases during cDNA synthesis thus producing cDNA products having sequence errors. RTs have in fact been shown to incorporate one base error per 3000-6000 nucleotides for HIV RT, and 1/10,000 nucleotide for AMV RT during cDNA synthesis (Berger, S. L., et al., Biochemistry 22:2365-2372 (1983); Krug, M. S., and Berger, S. L., Meth. Enzymol. 152:316 (1987); Berger et al. Meth. Enzymol. 275: 523 (1996)).

Scientists in the field have tried different enzyme compositions and methods for increasing the fidelity of polymerization on DNA or RNA templates. For example, Shevelev et al., Nature Rev. Mol. Cell Biol. 3:364 (2002) provides a review on 3′-5′ exonucleases. Perrino et al., PNAS, 86:3085 (1989) reports the use of epsilon subunit of E. coli DNA polymerase III to increase the fidelity of calf thymus DNA polymerase α. Bakhanashvili, Eur. J. Biochem. 268:2047 (2001) describes the proofreading activity of p53 protein and Huang et al., Oncogene, 17:261 (1998) describes the ability of p53 to enhance DNA replication fidelity. Bakhanashvili, Oncogene, 20:7635 (2001) later reports that p53 enhances the fidelity of DNA synthesis by HIV type I reverse transcriptase. Hawkins et al. describes the synthesis of full length cDNA from long mRNA transcript (2002, Biotechniques, 34:768).

U.S. patent application 2003/0198944A1 and U.S. Pat. No. 6,518,019 provide an enzyme mixture containing two or more reverse transcriptases (e.g., each reverse transcriptase having a different transcription pause site) and optionally one or more DNA polymerases. U.S. patent application 2002/0119465A1 discloses a composition that includes a mutant thermostable DNA polymerase and a mutant reverse transcriptase (e.g., a mutant Taq DNA polymerase and a mutant MMLV-RT). U.S. Pat. No. 6,485,917B1 and U.S. patent application 2003/0077762 and EP patent application EP1132470 provide a method for synthesizing cDNA in the presence of an enzyme having a reverse transcriptional activity and an α-type DNA polymerase having a 3′-5′ exonuclease activity.

Removal of the RNase H activity of RT can improve the efficiency of reverse transcription (Gerard, G. F., et al., FOCUS 11(4):60 (1989); Gerard, G. F., et al., FOCUS 14(3):91 (1992)). However such RTs (“RNase H⁻” forms) do not improve the fidelity of reverse transcription.

There is a need in the art for a composition and method to synthesize a cDNA with high efficiency and high fidelity.

### SUMMARY OF INVENTION

The present invention provides a composition comprising a first enzyme exhibiting a reverse transcriptase activity and a second enzyme exhibiting a 3′-5′ exonuclease activity, where the second enzyme exhibiting a 3′-5′ exonuclease activity comprises an epsilon subunit from an eubacteria.

In one embodiment, the second enzyme is thermostable.

In another embodiment, the second enzyme is thermolabile.

Preferably, the epsilon subunit is from E. coli.

In one embodiment, the first enzyme exhibiting a reverse transcriptase activity is a DNA polymerase.

Preferably, the DNA polymerase is a mutant DNA polymerase with an increased reverse transcriptase activity.

In another embodiment, the first enzyme exhibiting a reverse transcriptase activity is a reverse transcriptase (RT).

Preferably, the reverse transcriptase (RT) is a virus reverse transcriptase selected from the group consisting of: Moloney Murine Leukemia Virus (M-MLV) RT, Human Immunodeficiency Virus (HIV) RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus UR2AV RT, Avian Sarcoma Virus Y73 Helper Virus YAV RT, Rous Associated Virus (RAV) RT, and Myeloblastosis Associated Virus (MAV) RT.

More preferably, the reverse transcriptase is M-MLV reverse transcriptase or AMV reverse transcriptase.

Still more preferably, the reverse transcriptase is a reverse transcriptase with reduced RNase H activity.

In one embodiment, the reverse transcriptase with reduced RNase H activity is an M-MLV reverse transcriptase with reduced RNase H activity or an AMV reverse transcriptase with reduced RNase H activity.

In one embodiment, the first enzyme of the subject composition comprises an M-MLV reverse transcriptase with reduced RNase H activity and the second enzyme comprises E. coli DNA polymerase III epsilon subunit.

Preferably, the M-MLV reverse transcriptase with reduced RNase H activity is added at a working amount of 0.1-500 units per 20 μl reaction.

More preferably, the M-MLV reverse transcriptase with reduced RNase H activity is added at a working amount of 10-50 units per 20 μl reaction.

More preferably, the M-MLV reverse transcriptase with reduced RNase H activity is added at a working amount of 20-40 units per 20 μl reaction.

Preferably, the E. coli DNA polymerase III epsilon subunit is added at a working amount of 0.001-50 units per 20 μl reaction.

More preferably, the E. coli DNA polymerase III epsilon subunit is added at a working amount of 0.01-25 units units per 20 μl reaction.

More preferably, the E. coli DNA polymerase III epsilon subunit is added at a working amount of 0.01-10 units per 20 μl reaction.

The invention provides a kit for cDNA synthesis comprising a first enzyme exhibiting a reverse transcriptase activity with reduced RNase H activity and a second enzyme exhibiting a 3′-5′ exonuclease activity and packaging materials therefor, where the second enzyme exhibiting a 3′-5′ exonuclease activity comprises an epsilon subunit from an eubacteria.

Preferably, the epsilon subunit of the subject kit is from E. coli.

In a preferred embodiment, the first enzyme of the subject kit comprises M-MLV reverse transcriptase with reduced RNase H activity and the second enzyme comprises E. coli DNA polymerase III epsilon subunit.

Preferably, the M-MLV reverse transcriptase with reduced RNase H activity is added at a working amount of 20-40 units per 20 μl reaction.

Preferably, the E. coli DNA polymerase III epsilon subunit is added at a working amount of 0.01-10 units per 20 μl reaction.

The subject kit may further comprise one or more of components selected from the group consisting of: one or more oligonucleotide primers, one or more nucleotides, a suitable buffer, one or more PCR accessory factors, and one or more terminating agents.

The present invention provides a method for cDNA synthesis comprising: (a) contacting one or more nucleic acid templates with an enzyme composition comprising a first enzyme exhibiting a reverse transcriptase activity with reduced RNase H activity and a second enzyme exhibiting a 3′-5′ exonuclease activity, where the second enzyme exhibiting a 3′-5′ exonuclease activity comprises an epsilon subunit from an eubacteria; and (b) incubating the templates and the enzyme composition under conditions sufficient to permit cDNA synthesis.

The subject method of the present invention may further comprise (c) incubating the synthesized cDNA under conditions sufficient to make one or more nucleic acid molecules complementary the cDNA.

The invention also provides a method for amplifying one or more nucleic acid molecules, the method comprising: (a) contacting one or more nucleic acid templates with an enzyme composition comprising a first enzyme exhibiting a reverse transcriptase activity with reduced RNase H activity and a second enzyme exhibiting a 3′-5′ exonuclease activity, where the second enzyme exhibiting a 3′-5′ exonuclease activity comprises an epsilon subunit from an eubacteria; and (b) incubating the templates and the enzyme composition under conditions sufficient to permit amplification of one or more nucleic acid molecules.

Preferably, the nucleic acid template is a messenger RNA molecule or a population of MRNA molecules.

In one embodiment, the second enzyme used in the subject method is the epsilon subunit from E. coli.

### BRIEF DESCRIPTION OF FIGURES

FIG. 1 shows the nucleotide sequences of regulatory sequence and primer sequences used in a reverse transcription reaction according to one embodiment of the invention.

FIG. 2 shows the result of RT-PCR using M-MLV RT with reduced RNase H activity in combination with different enzymes exhibiting 3′-5′ exonuclease activity according to one embodiment of the invention.

FIG. 3 shows the RT-PCR result using M-MLV RT with reduced RNase H activity and various amount of epsilon subunit of E. coli DNA polymerase III in amplifying a 4 kb and a 6 kb template polynucleotide according to one embodiment of the present invention.

FIG. 4 shows the RT-PCR result using AMV RT and various amount of epsilon subunit of E. coli DNA polymerase III in amplifying a 4 kb template polynucleotide according to one embodiment of the present invention.

FIGS. 5A-ZZ show nucleotide and amino acid sequences for various useful epsilon subunits according to one embodiment of the invention. The Figures also show the amino acid sequence alignments for various epsilon subunits.

FIG. 6 shows the nucleotide and amino acid sequences for epsilon subunit from E. coli DNA polymerase III and Thermatoga maritima DNA polymerase III according to one embodiment of the invention.

### DETAILED DESCRIPTION OF THE INVENTION

Definitions

As used herein, “polynucleotide polymerase” refers to an enzyme that catalyzes the polymerization of nucleotides, e.g., to synthesize polynucleotide strands from ribonucleoside triphosphates or deoxynucleoside triphosphates. Generally, the enzyme will initiate synthesis at the 3′-end of a primer annealed to a polynucleotide template sequence, and will proceed toward the 5′ end of the template strand. “DNA polymerase” catalyzes the polymerization of deoxynucleotides to synthesize DNA, while “RNA polymerase” catalyzes the polymerization of ribonucleotides to synthesize RNA.

The term “DNA polymerase” refers to a DNA polymerase which synthesizes new DNA strands by the incorporation of deoxynucleoside triphosphates in a template dependent manner (i.e., having a DNA polymerase activity). One unit of DNA polymerase activity of a DNA polymerase, according to the subject invention, is defined as the amount of the enzyme which catalyzes the incorporation of 10 nmoles of total deoxynucleotides (dNTPs) into polymeric form in 30 minutes at optimal temperature. The measurement of DNA polymerase activity may be performed according to assays known in the art, for example, as described by a previously published method (Hogrefe, H. H., et al (01) Methods in Enzymology, 343:91-116) and as described in DNA Replication 2nd Ed., Komberg and Baker, supra; Enzymes, Dixon and Webb, Academic Press, San Diego, Calif. (1979). A “DNA polymerase” may be DNA-dependent (i.e., using a DNA template) or RNA-dependent (i.e., using a RNA template). It is intended that the term encompass any DNA polymerases known in the art, e.g., as described herein below. Both thermostable and thermnolabile are encompassed by this definition.

As used herein, the term “reverse transcriptase (RT)” is used in its broadest sense to refer to any enzyme that exhibits reverse transcription activity as measured by methods disclosed here or known in the art. A “reverse transcriptase” of the present invention, therefore, includes reverse transcriptases from retroviruses, other viruses, and bacteria, as well as a DNA polyrnerase exhibiting reverse transcriptase activity, such as Tth DNA polymerase, Taq DNA polymerase, Tne DNA polymerase, Tma DNA polymerase, etc. RT from retroviruses include, but are not limited to, Moloney Murine Leukemia Virus (M-MLV) RT, Human Immunodeficiency Virus (HIV) RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus UR2AV RT, Avian Sarcoma Virus Y73 Helper Virus YAV RT, Rous Associated Virus (RAV) RT, and Myeloblastosis Associated Virus (MAV) RT, and as described in U.S. patent application 2003/0198944 (hereby incorporated by reference in its entirety). For review, see e.g. Levin, 1997, Cell, 88:5-8; Brosius et al., 1995, Virus Genes 11:163-79. Known reverse transcriptases from viruses require a primer to synthesize a DNA transcript from an RNA template. Reverse transcriptase has been used primarily to transcribe RNA into cDNA, which can then be cloned into a vector for further manipulation or used in various amplification methods such as polymerase chain reaction (PCR), nucleic acid sequence-based amplification (NASBA), transcription mediated amplification (TMA), or self-sustained sequence replication (3SR).

As used herein, the terms “reverse transcription activity” and “reverse transcriptase activity” are used interchangeably to refer to the ability of an enzyme (e.g., a reverse transcriptase or a DNA polymerase) to synthesize a DNA strand (i.e., cDNA) utilizing an RNA strand as a template. Methods for measuring RT activity are provided herein below and also are well known in the art. For example, the Quan-T-RT assay system is commercially available from Amersham (Arlington Heights, Ill.) and is described in Bosworth, et al., Nature 1989, 341:167-168. A “first enzyme,” according to the present invention, is a purified or isolated enzyme containing a detectable reverse transcriptase activity using methods known in the art. The “first enzyme,” of the present invention, therefore, may be a reverse transcriptase from a retrovirus or a DNA polymerase exhibiting a reverse trsnacriptase activity.

As used herein, the term “increased” reverse transcriptase activity refers to the level of reverse transcriptase activity of a mutant enzyme (e.g., a DNA polymerase) as compared to its wild-type form. A mutant enzyme is said to have an “increased” reverse transcriptase activity if the level of its reverse transcriptase activity (as measured by methods described herein or known in the art) is at least 20% or more than its wild-type form, for example, at least 25%, 30%, 40%, 50%, 60%, 70%, 80%, 90%, 100% more or at least 2-fold, 3-fold, 4-fold, 5-fold, or 10-fold or more.

As used herein, “exonuclease” refers to an enzyme that cleaves bonds, preferably phosphodiester bonds, between nucleotides one at a time from the end of a DNA molecule. An exonuclease can be specific for the 5′ or 3′ end of a DNA molecule, and is referred to herein as a 5′ to 3′ exonuclease or a 3′ to 5′ exonuclease. The 3′ to 5′ exonuclease degrades DNA by cleaving successive nucleotides from the 3′ end of the polynucleotide while the 5′ to 3′ exonuclease degrades DNA by cleaving successive nucleotides from the 5′ end of the polynucleotide. During the synthesis or amplification of a polynucleotide template, a DNA polymerase with 3′ to 5′ exonuclease activity (3′ to 5′ exo⁺) has the capacity of removing mispaired base (proofreading activity), therefore is less error-prone (i.e., with higher fidelity) than a DNA polymerase without 3′ to 5′ exonuclease activity (3′ to 5′ exo⁻). A “second enzyme,” according to the present invention, is a purified or isolated enzyme comprising a detectable 3′-5′ exonuclease activity using methods known in the art, e.g., as described herein. The “second enzyme,” of the present invention may be a holoenzyme containing 3′-5′ exonuclease activity or it may be an enzyme containing one or more subunits of the holoenzyme which possesses 3′-5′ exonuclease activity. A non-limiting example of holoenzymes is E. coli DNA polymerase III, and a non-limiting example of an enzyme containing a subunit posessing 3′-5′ exonuclease activity is the epsilon subunit of E. coli DNA polymerase III. The exonuclease activity can be measured by methods well known in the art, and as described below. For example, one unit of exonuclease activity may refer to the amount of enzyme that hydrolyze 1 nmole of pNP-TMP per minute at pH8 and 25° C., or as described in Hamdan, S. et al. (Biochemistry 2002, 41:5266-5275, hereby incorporated in its entirety).

The term “E. coli DNA polymerase III holoenzyme” refers to a E. coli polymerase III holoenzyme composed of ten subunits assembled in two catalytic cores, two sliding clamps and a clamp loader, e.g., as described in Kelman, Z. & O'Donnell, M. (1995). Annu. Rev. Biochem. 64, 171200 (the entirety is hereby incorporated by reference).

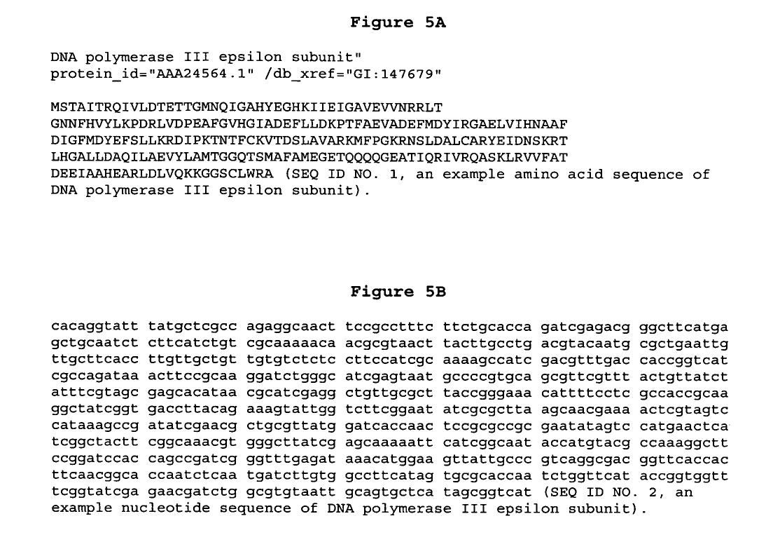

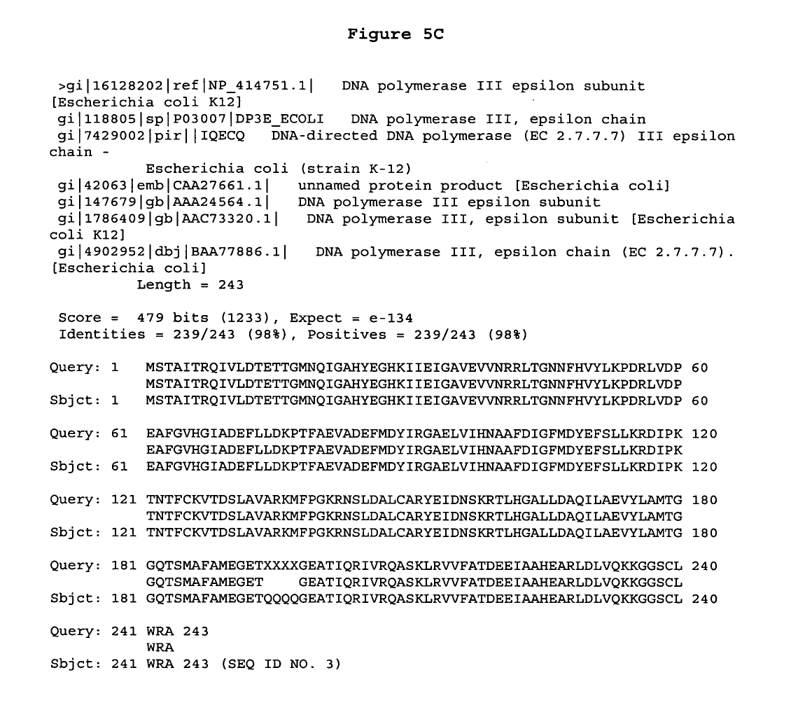

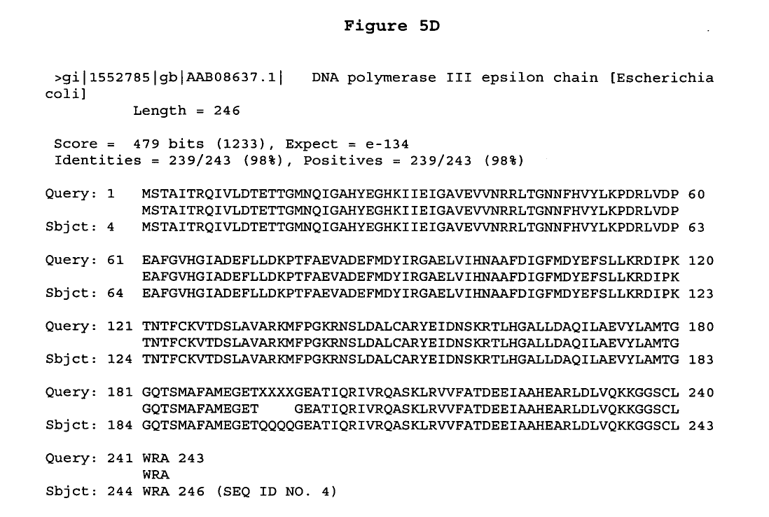

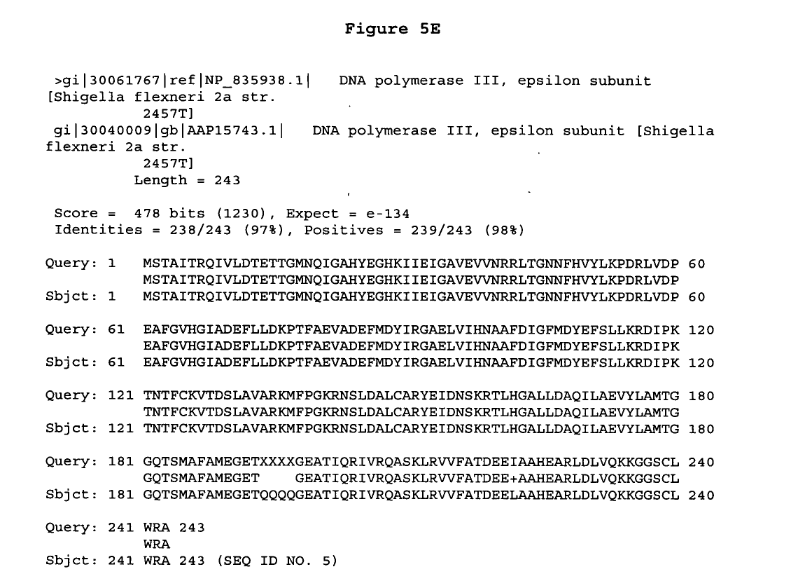

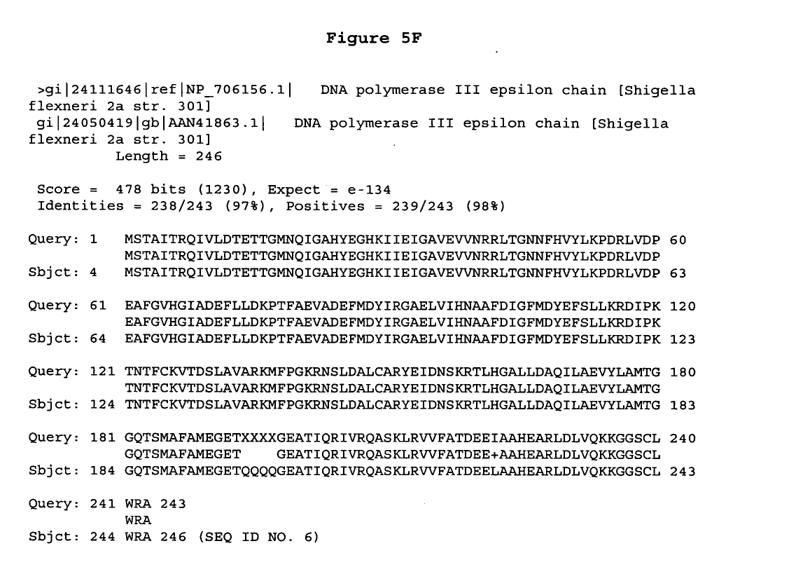

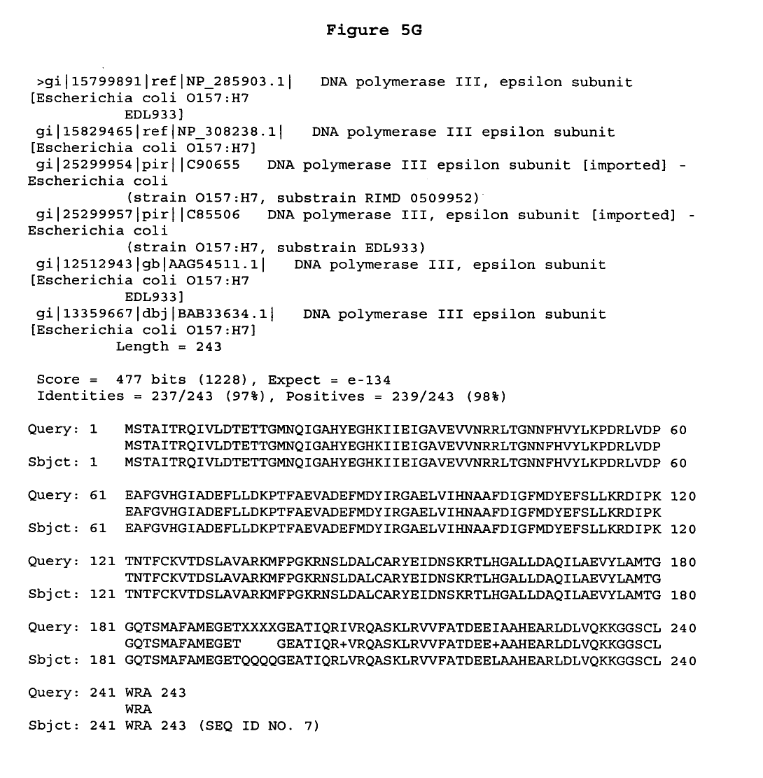

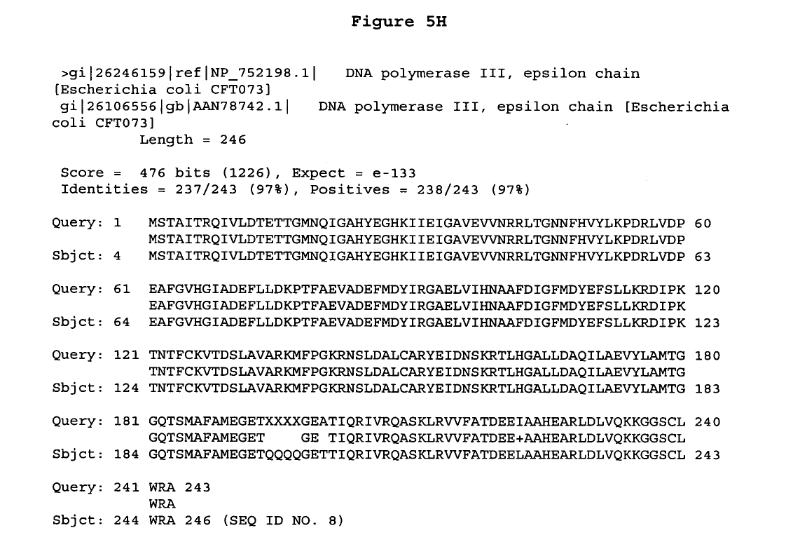

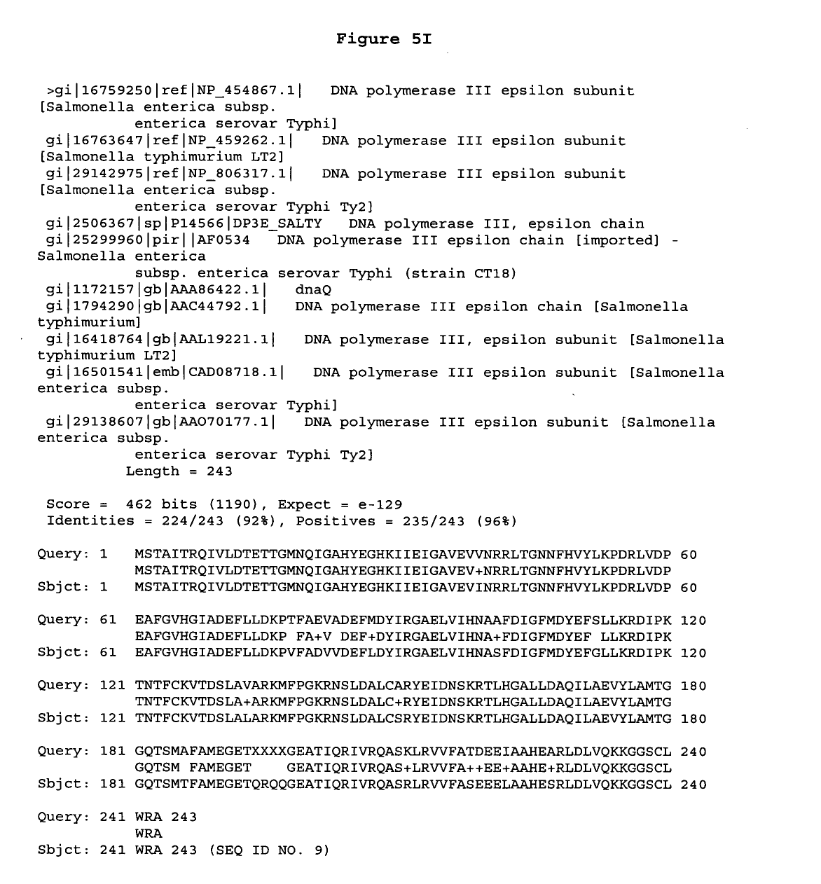

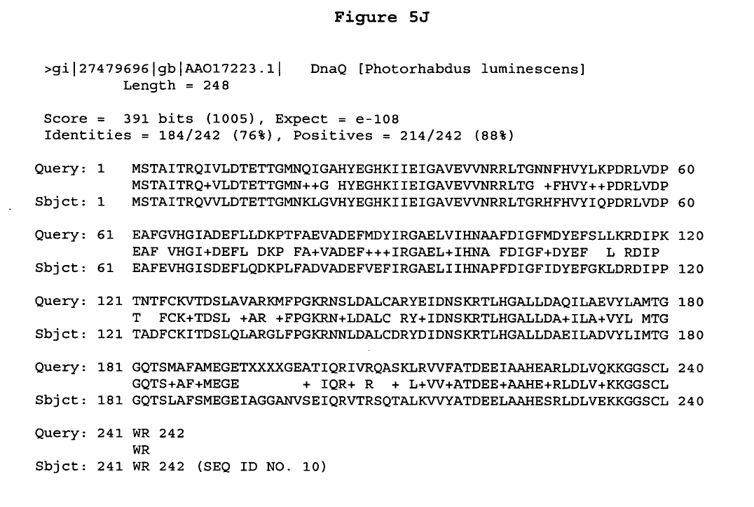

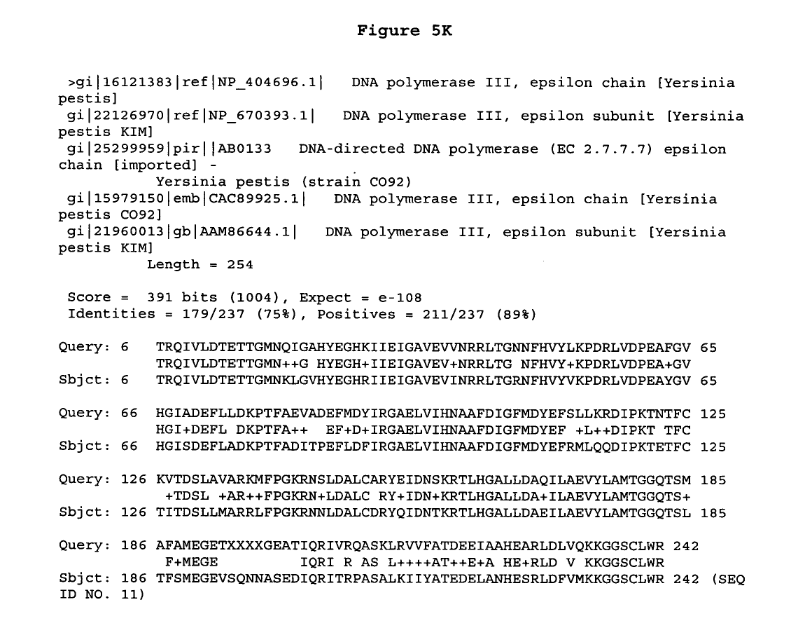

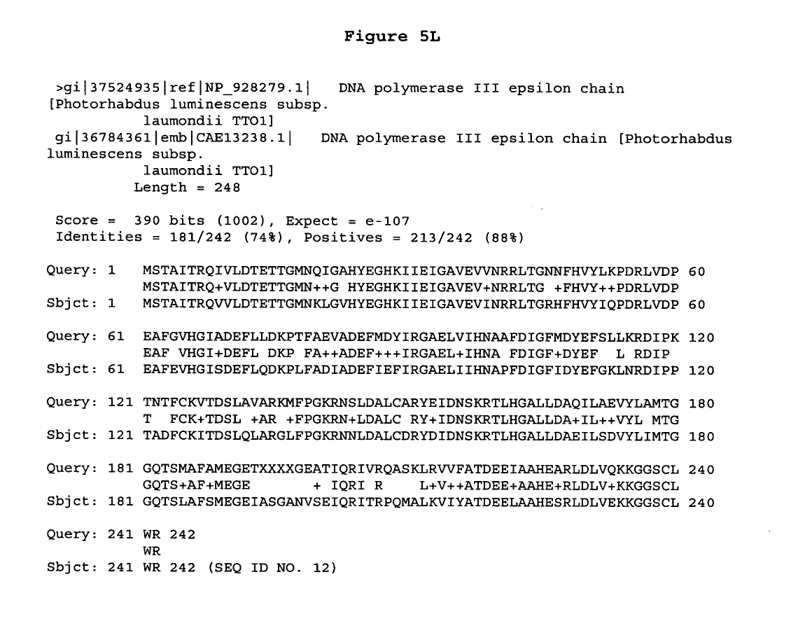

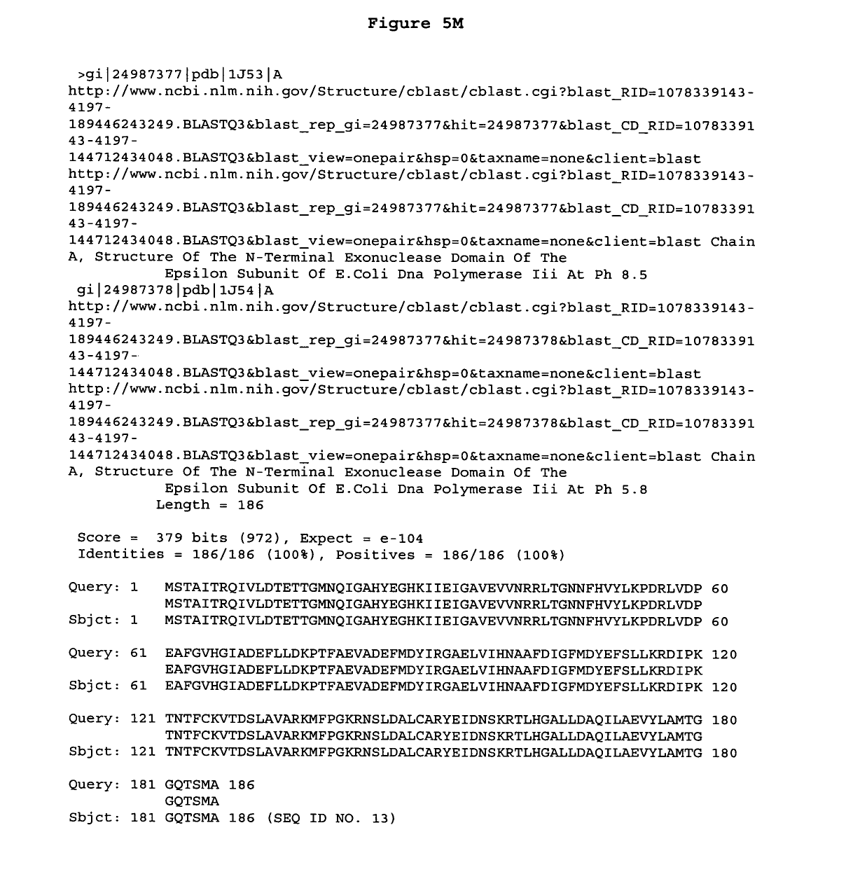

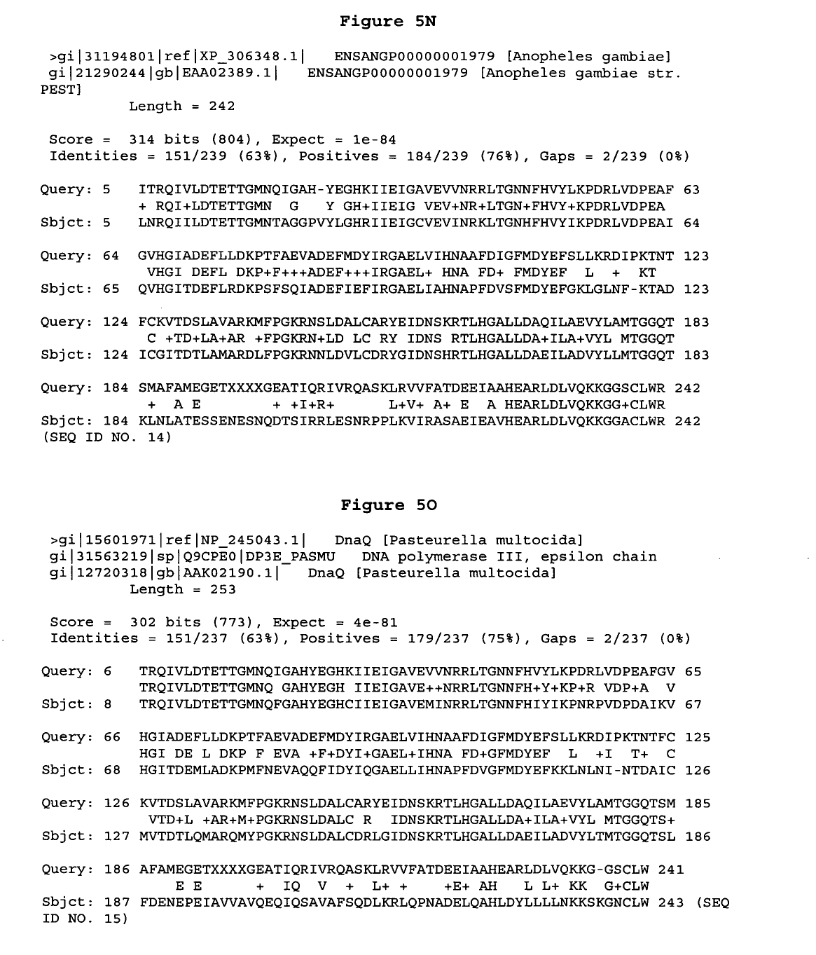

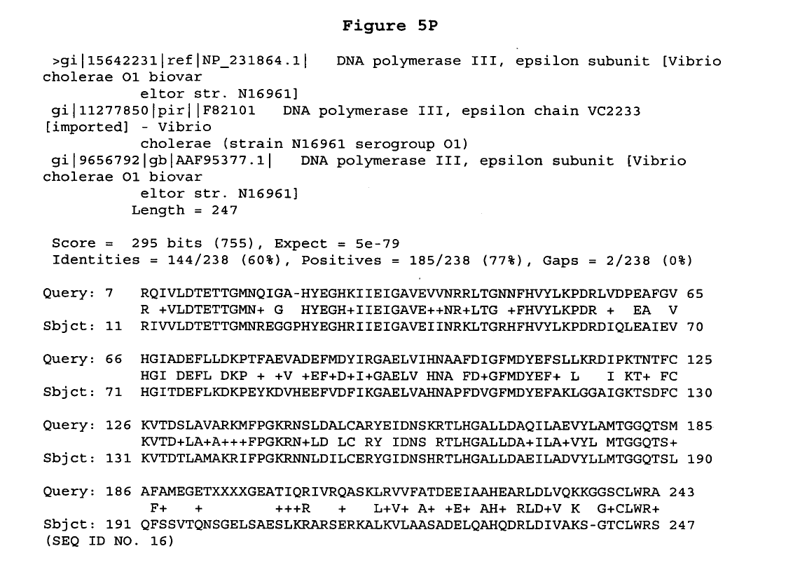

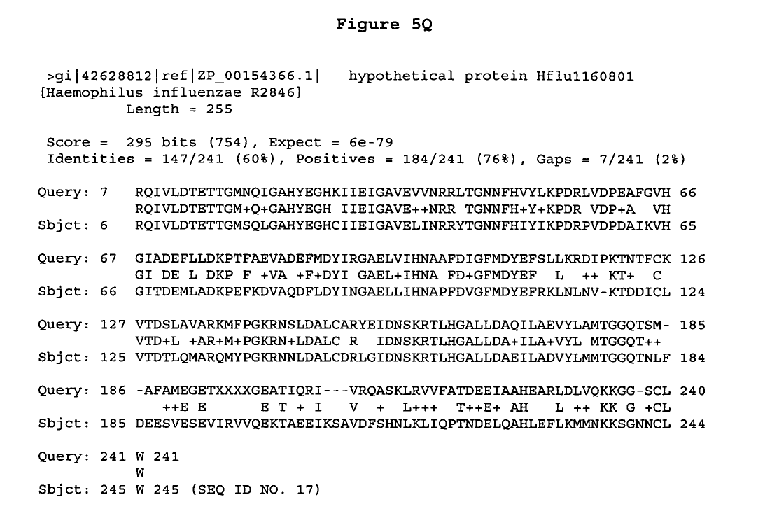

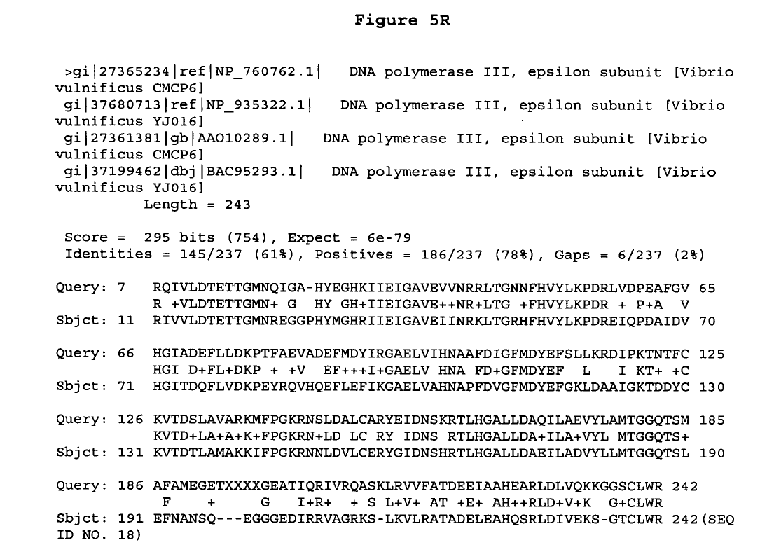

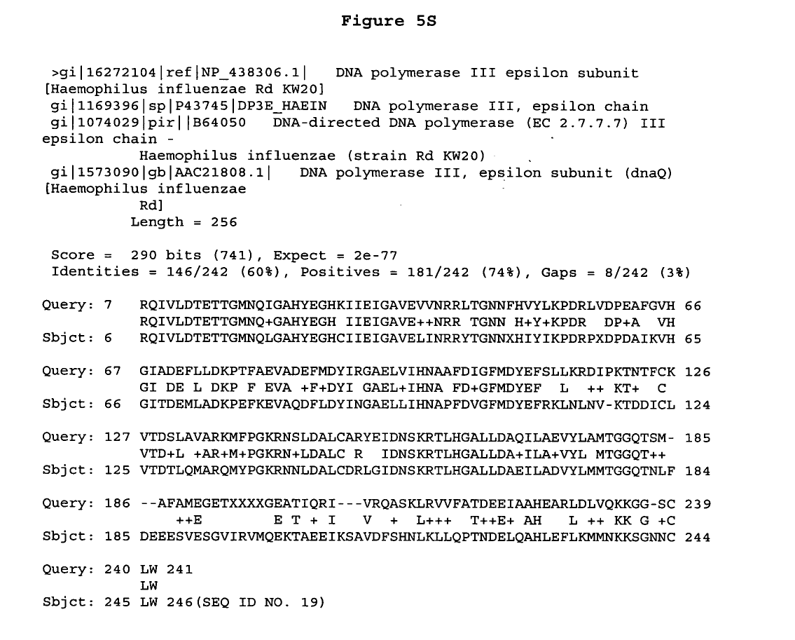

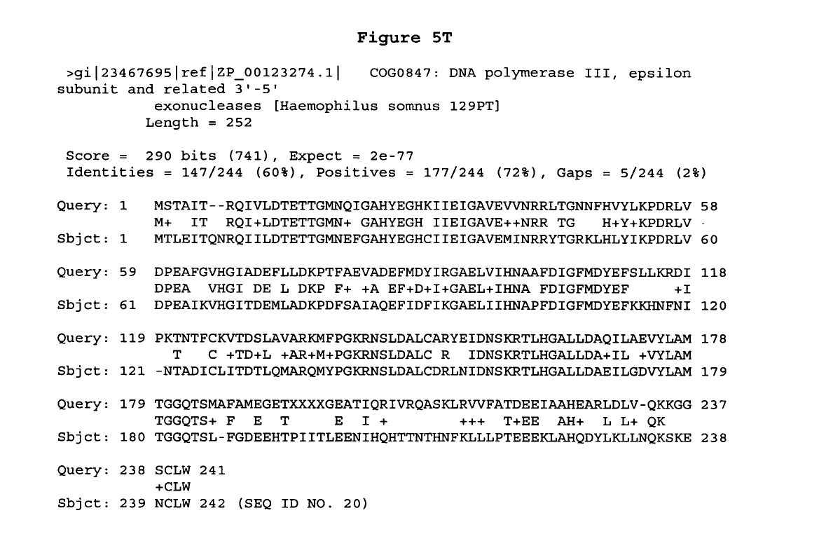

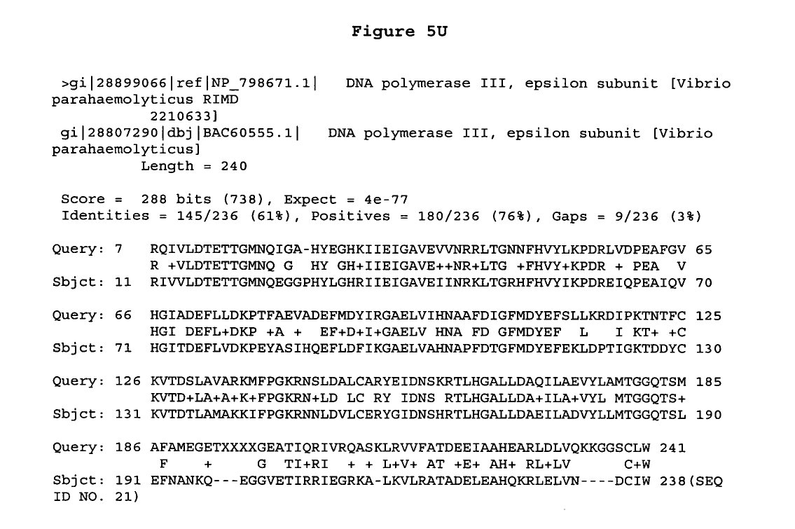

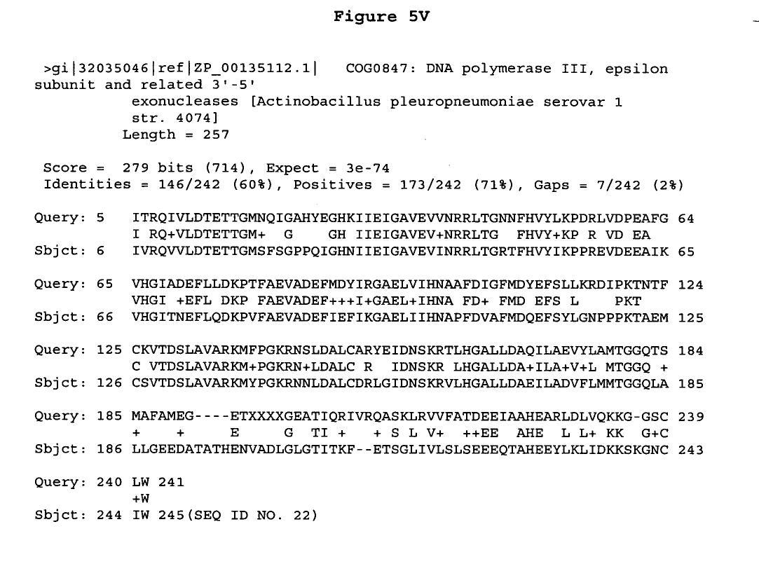

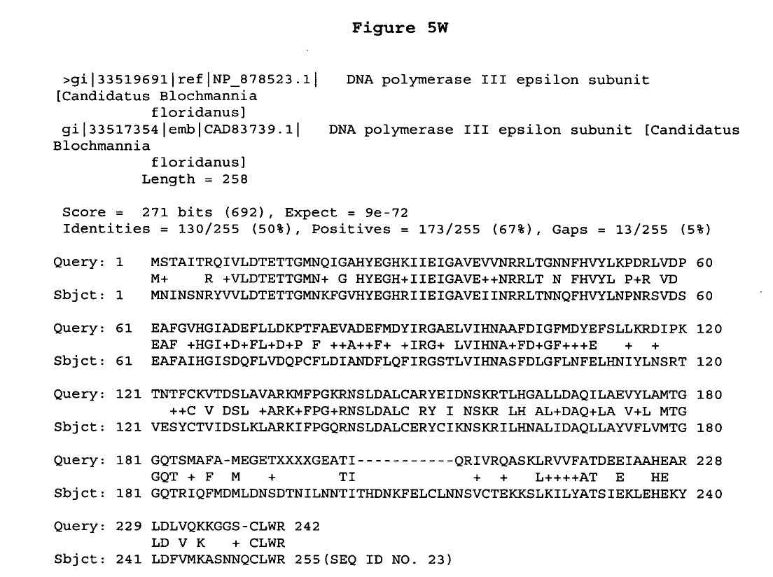

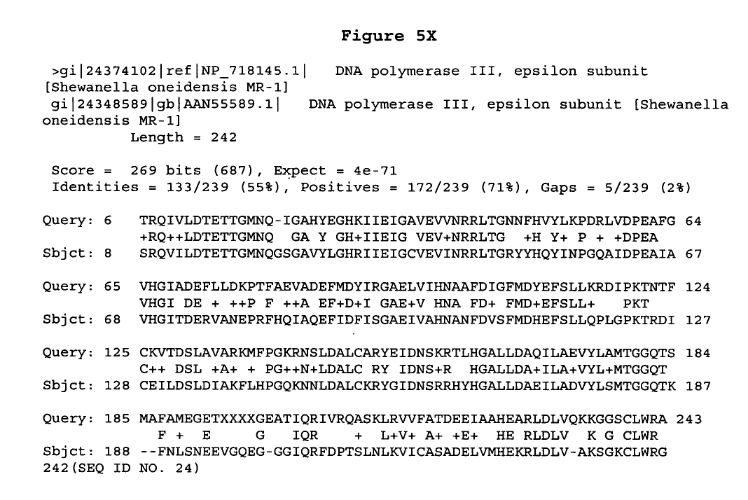

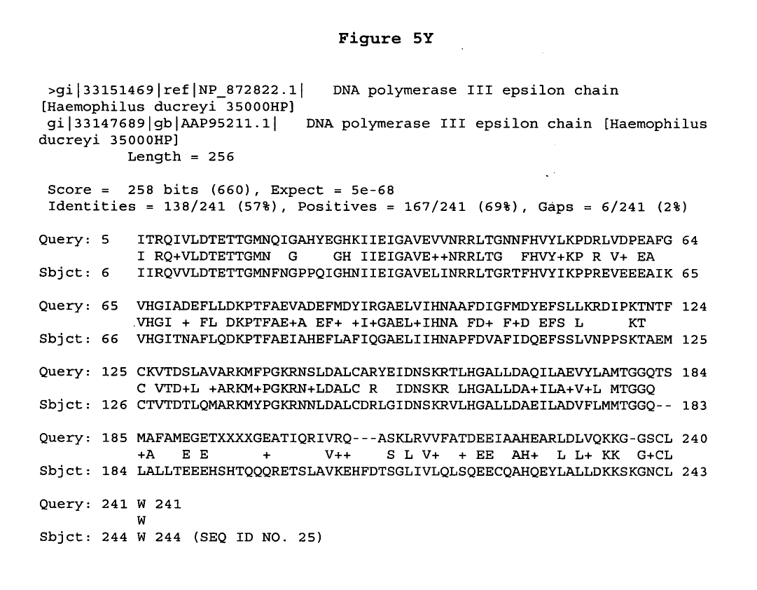

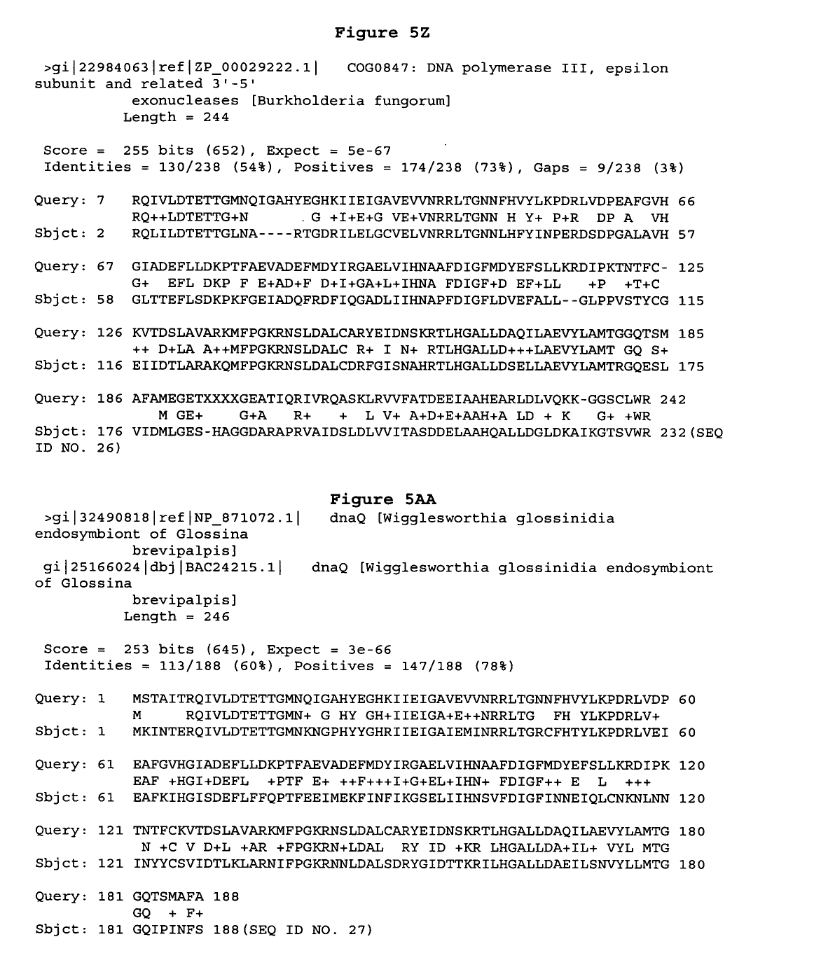

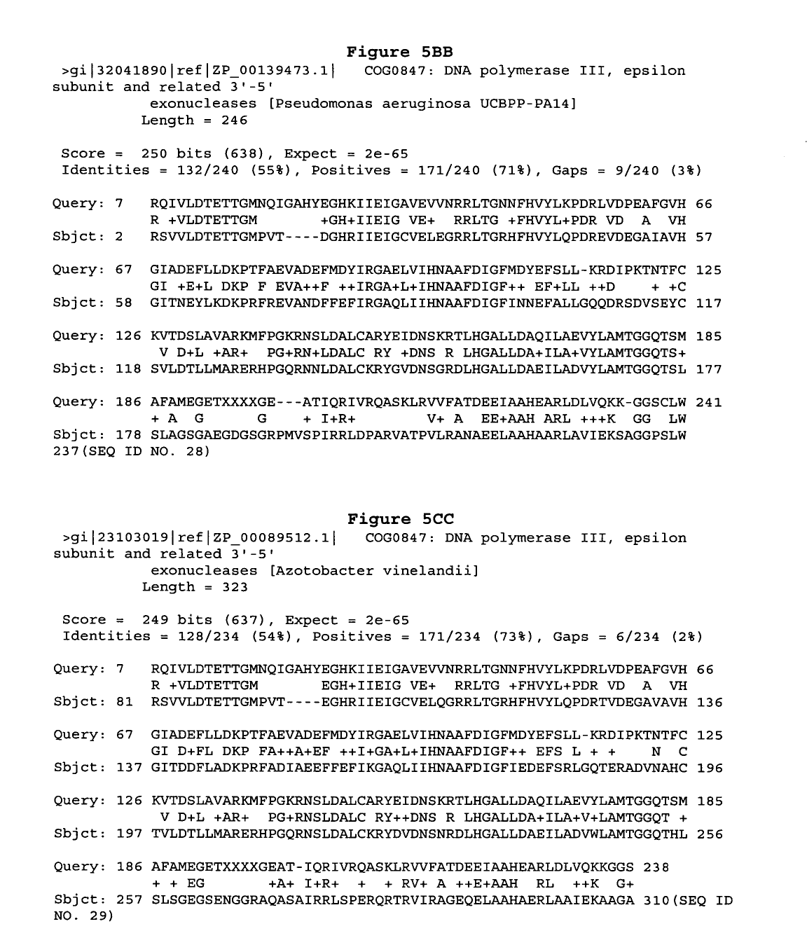

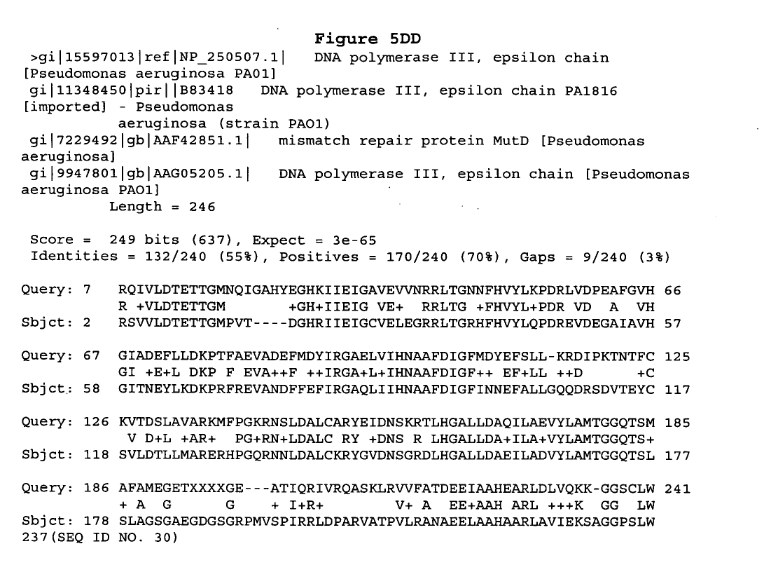

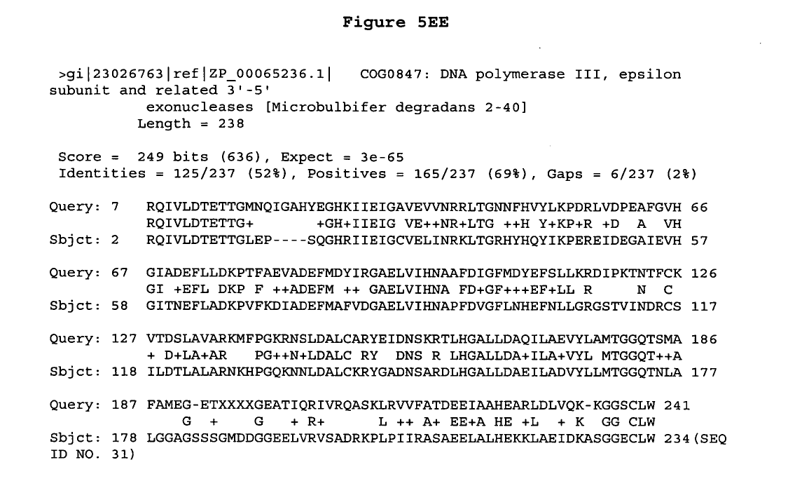

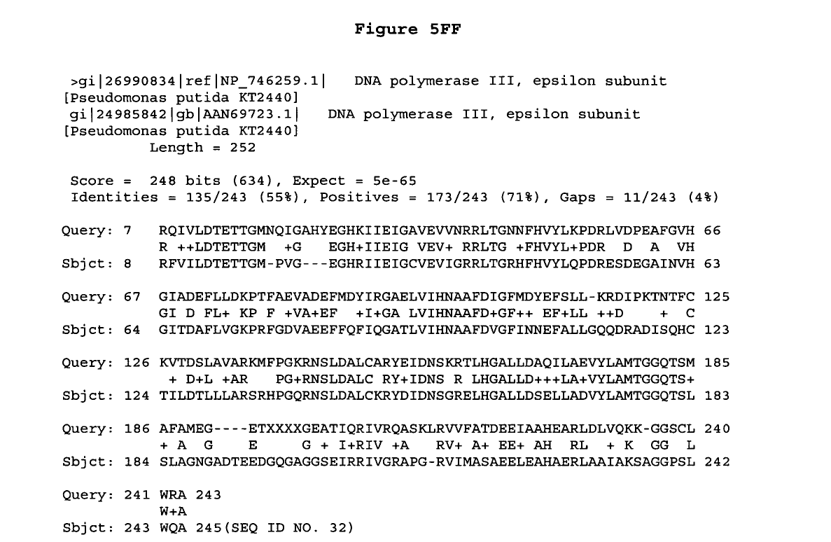

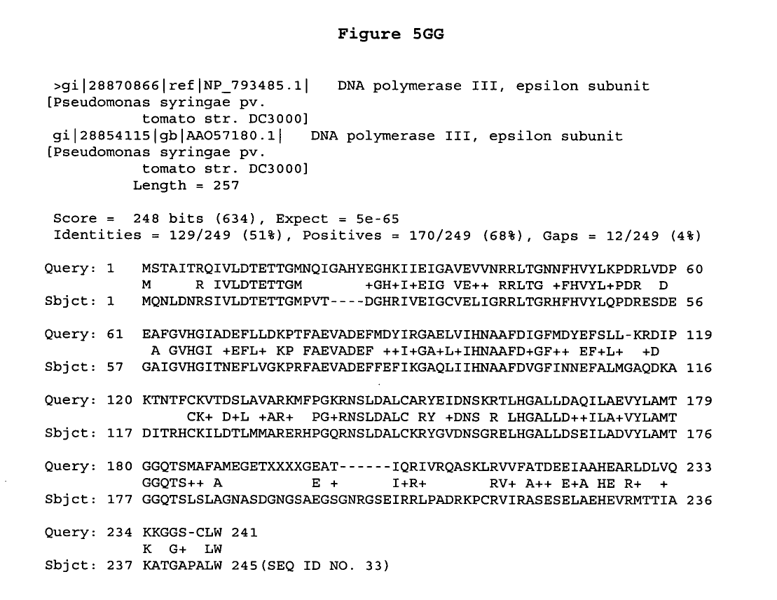

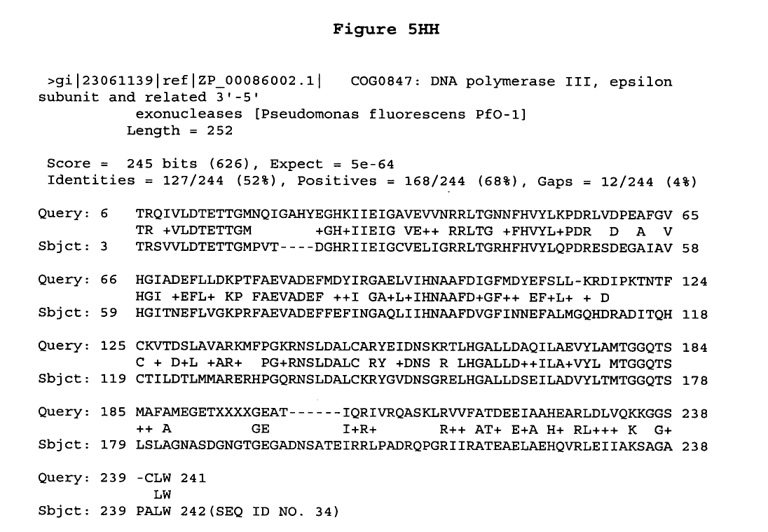

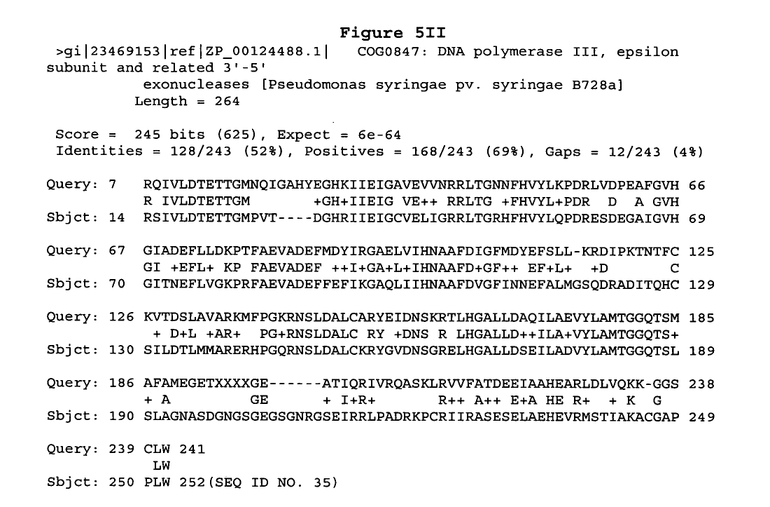

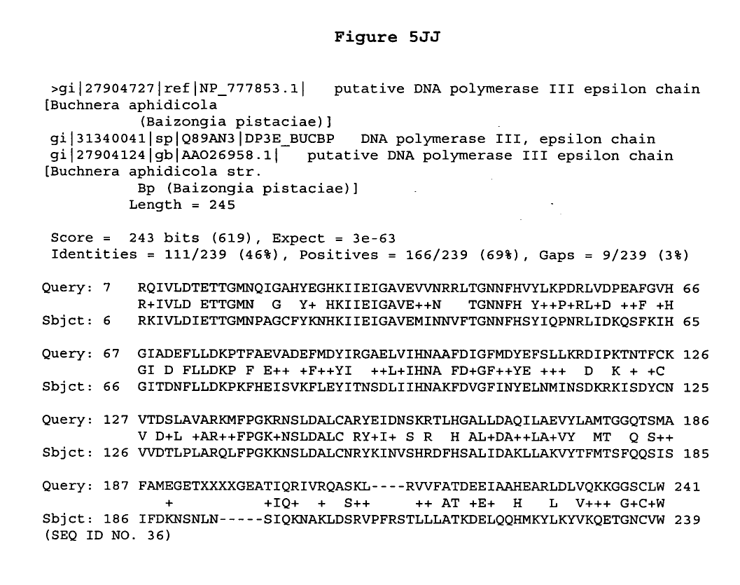

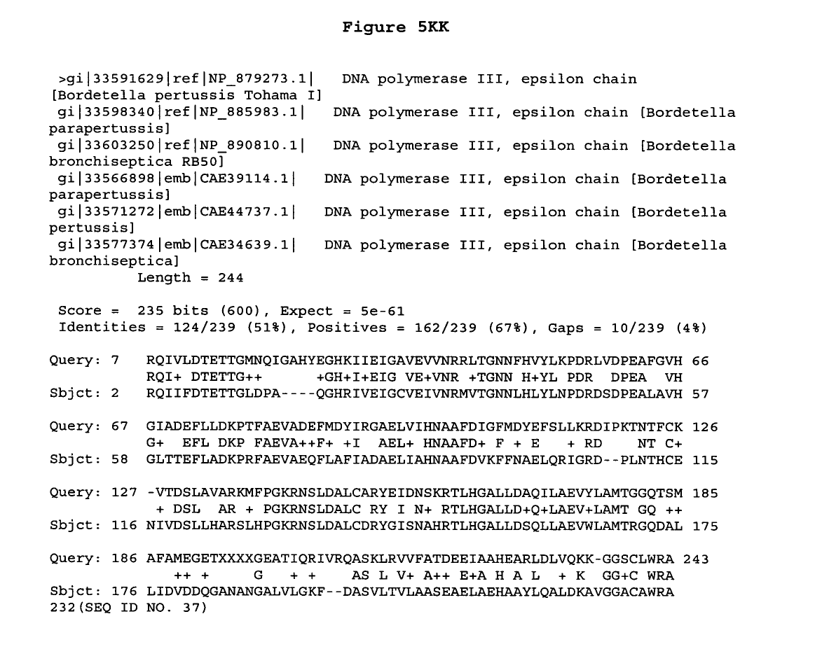

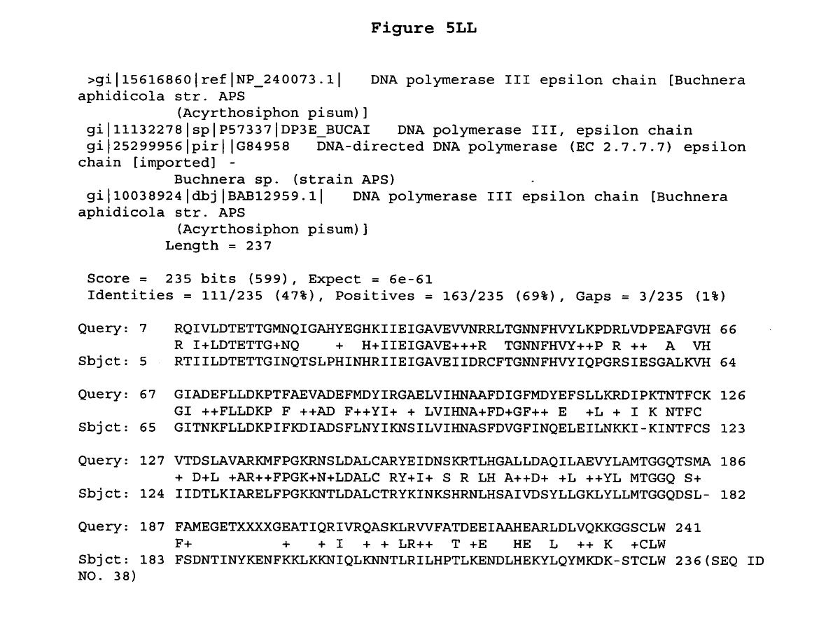

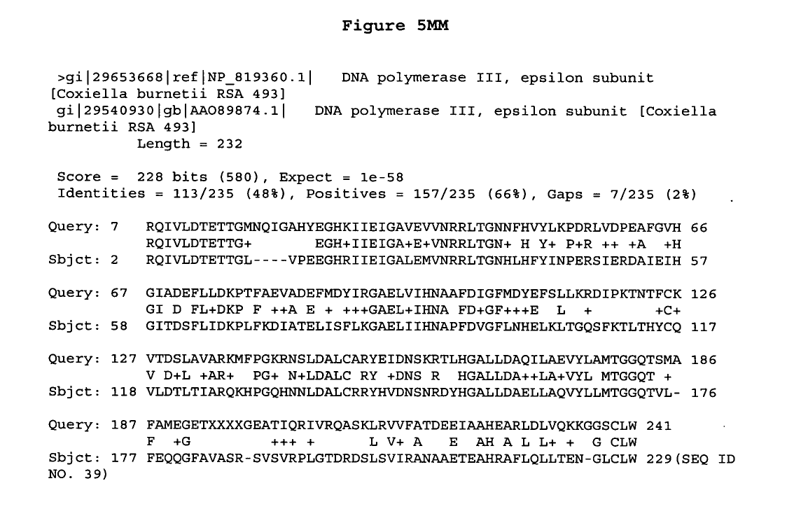

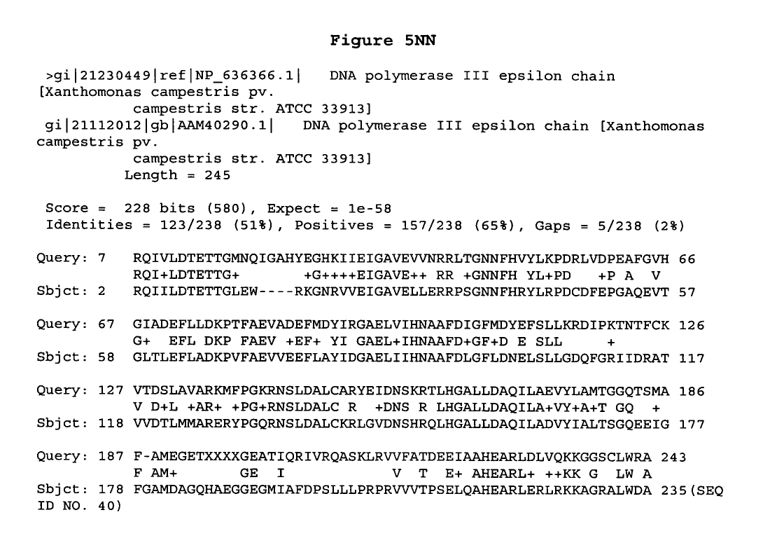

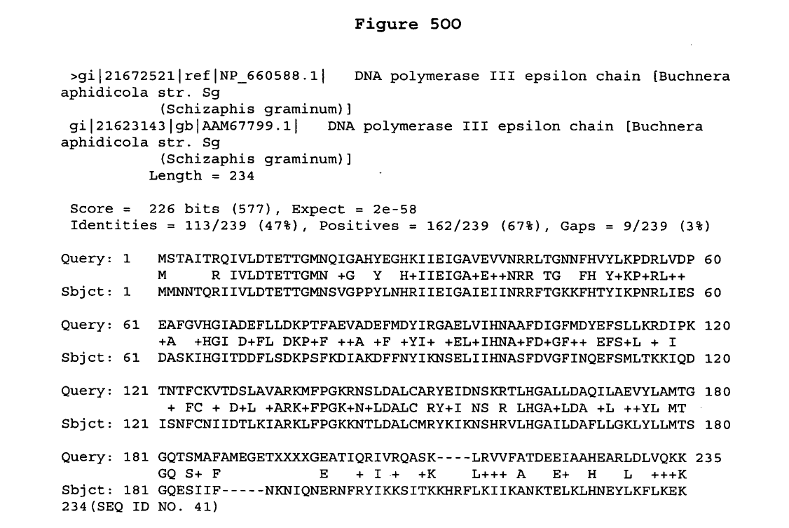

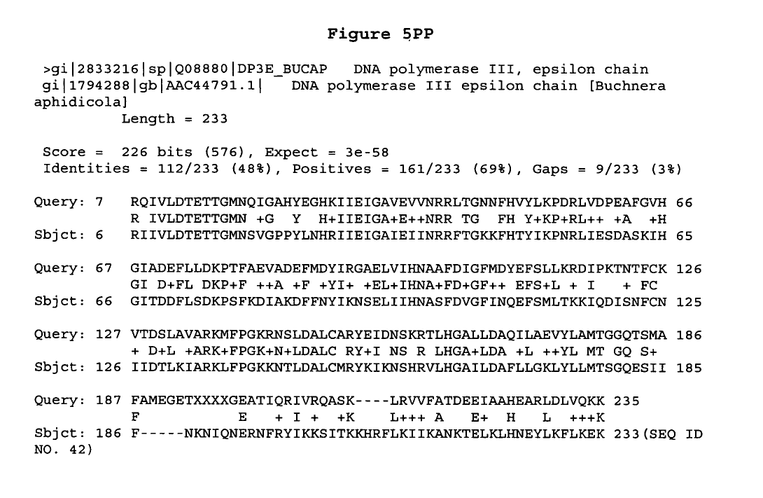

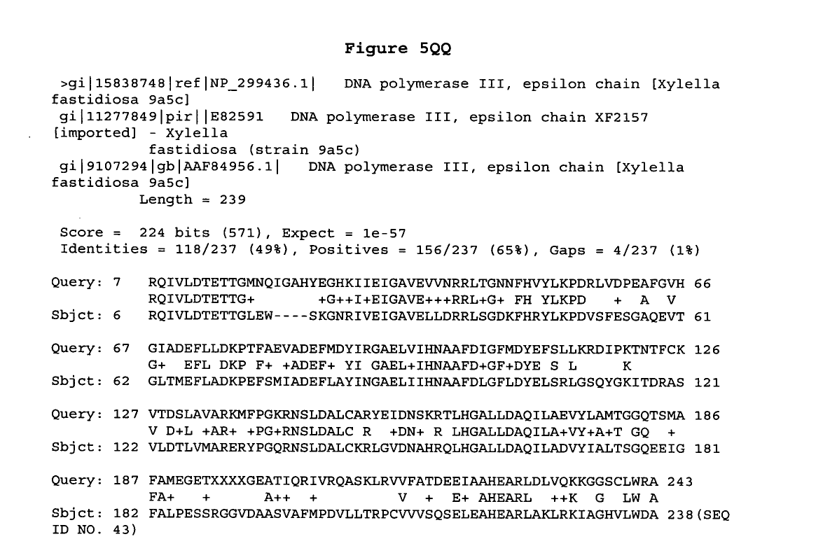

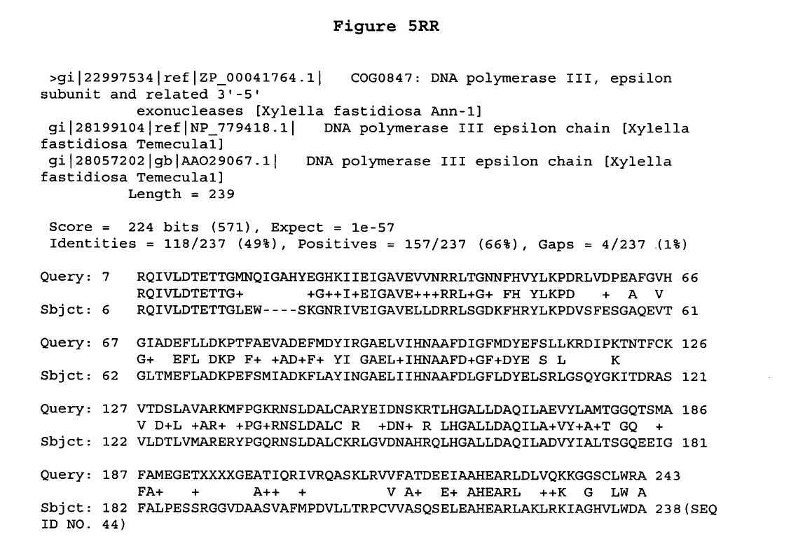

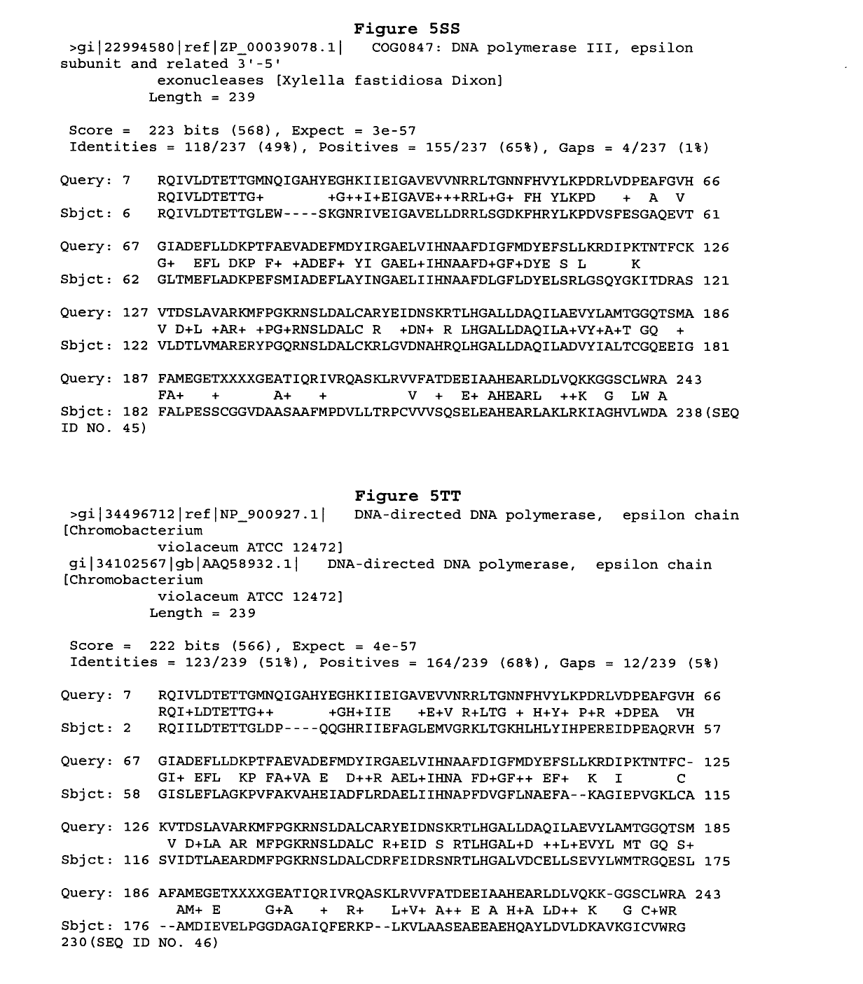

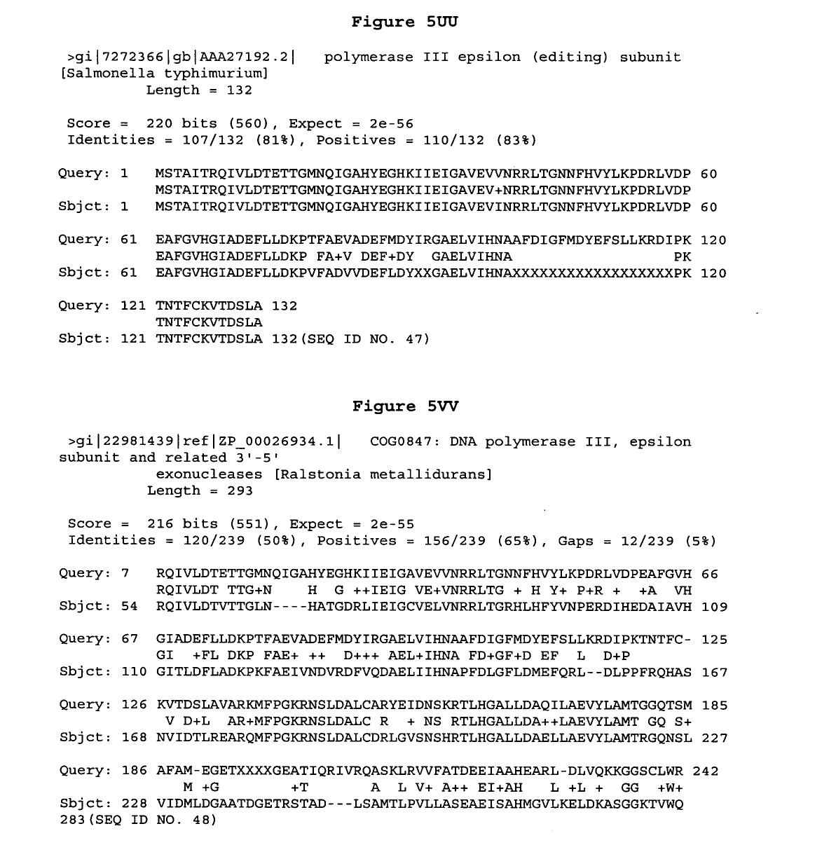

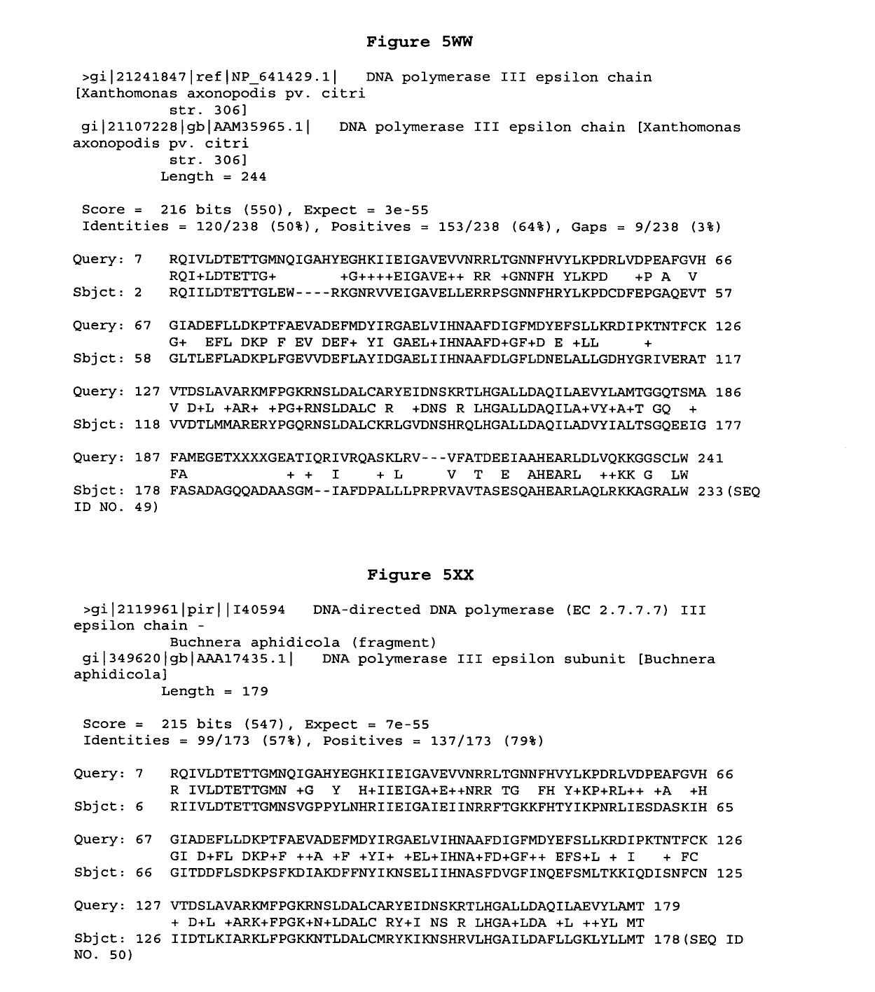

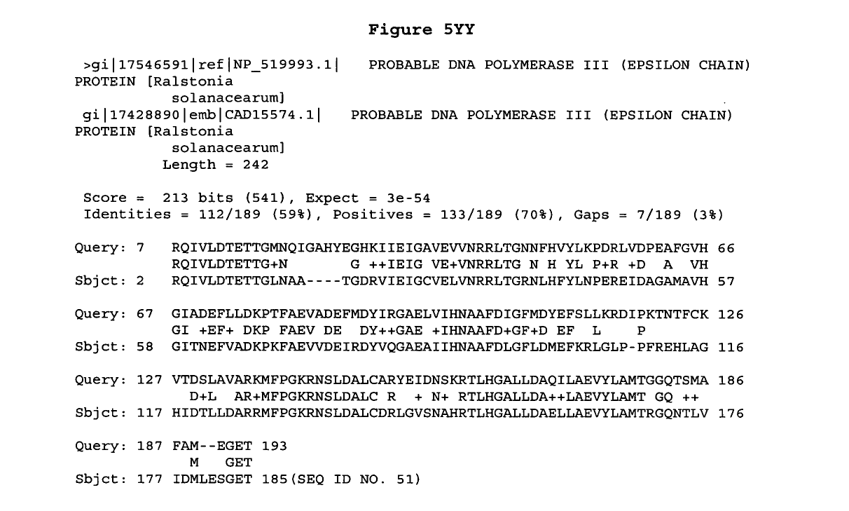

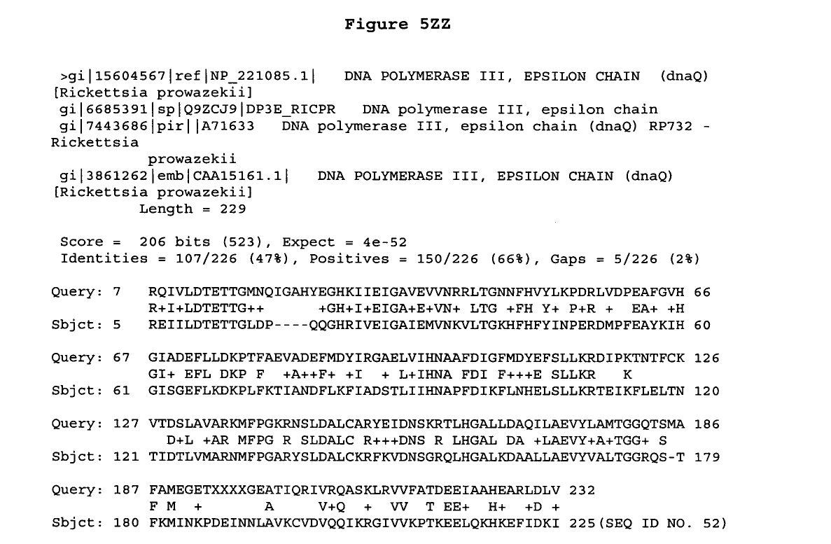

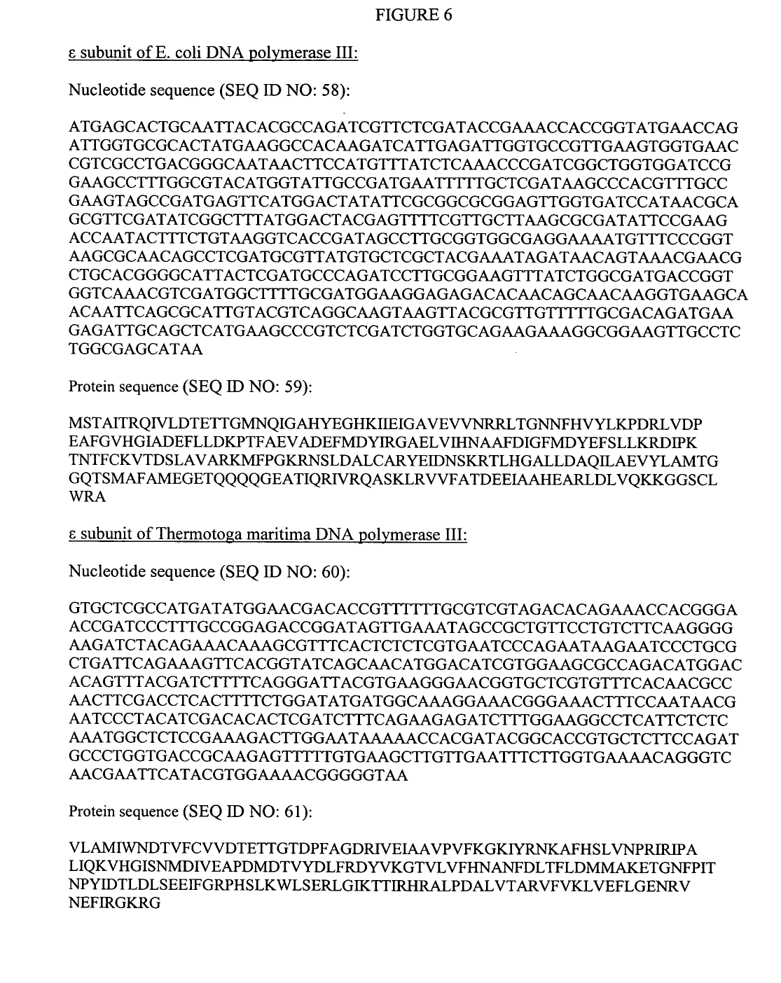

The term “epsilon (ε) subunit,” according to the present invention, refers to a ε subunit having 3′-5′ exonuclease activity. An epsilon subunit may be from any eubacteria, such as from E. coli, or from other organisms. The epsilon (ε) subunit of the E. coli DNA polymerase III holoenzyme is the 3′-5′ exonuclease of the holoenzyme and interacts with the α (polymerase unit) and θ (unknown function) subunits (see, e.g., Fijalkowska et al., 1996, Proc. Natl. Acad. Sci. USA, 93: 2856-2861, the entirety is hereby incorporated by reference). The epsilon (ε) subunit of E. coli DNA polymerase III holoenzyme (i.e., SEQ. ID NO:1) is encoded by dnaQ gene, e.g., SEQ. ID NO:2. The epsilon subunit of the present invention also include a wild type polypeptide which is at least 50% homologous (e.g., 60%, 70%, 80%, 90%, or identical) to SEQ. ID NO:1 and contains 3′-5′ exonuclease activity, e.g., as shown in FIGS. 5A-ZZ and FIG. 6. The epsilon (ε) subunit, according to the present invention, further include a mutant epsilon (ε) subunit which still contains 3′-5′ exonuclease activity. Such mutant epsilon may contain deletion (e.g., truncation), substitution, point mutation, mutation of multiple amino acids, or insertion to the wild type epsilon subunit. For example, a truncated epsilon useful according to the invention may be as what's disclosed in Hamdan S. et al., Biochemistry 2002, 41: 5266-5275, the entirety hereby incorporated by reference.

As used herein, the term “eubacteria” refers to unicelled organisms which are prokaryotes (e.g., as described in Garrity, et al., 2001, Taxonomic outline of the procaryotic genera. Bergey's Manual® of Systematic Bacteriology, Second Edition. Release 1.0, April 2001, and in Werren, 1997, Annual Review of Entomology 42: 587-609). Eubacteria include the following genera: Escherichia, Pseudomonas, Proteus, Micrococcus, Acinetobacter, Klebsiella, Legionella, Neisseria, Bordetella, Vibrio, Staphylococcus, Lactobaccilus, Streptococcus, Bacillus, Corynebacteria, Mycobacteria, Clostridium, and others (see Kandler, O., Zbl. Bakt.Hyg., I.Abt.Orig. C3, 149-160 (1982)), as well as major sub-groups of eubacteria such as Aquifex (extremely thermophilic chemolithotrophs), Thermotoga (extremely thermophilic chemoorganotrophs), Chloroflexus (thermophilic photosynthetic bacteria), Deinococcus (radiation resistant bacteria), Thermus (thermophilic chemoheterotrophs), Spirochaetes (helical bacteria with periplasmic flagella), Proteobacteria (Gram-negative and purple photosynthetic bacteria), Cyanobacteria (blue-green photosynthetic bacteria), Gram-positives (Gram-positive bacteria), Bacteroides/Flavobacterium (strict anaerobes/ strict aerobes with gliding motility), Chlorobium (photoautotrophic sulphur-oxidisers), Planctomyces (budding bacteria with no peptidoglycan), Chlamydia (intracellular parasites).

As used herein, a “blend,” according to the present invention, refers to a mix of two or more purified enzymes comprising at least a first enzyme and a second enzyme as described above. The blend may be in liquid or dry form. Each individual enzyme (e.g., the first enzyme or the second enzyme) in the blend may no longer exist as a “purified” or “isolated” enzyme as defined herein below.

As used herein, an enzyme composition “consisting essentially of E. coli polymerase III epsilon subunit and a reverse transcriptase” refers to an enzyme composition where its 3′-5′ exonuclease activity is substantially (i.e., at least 50%, e.g., 60%, 70%, 80%, 90%, or 100%) provided by E. coli polymerase III epsilon subunit.

The term “fidelity,” as used herein, refers to the accuracy of DNA synthesis by template-dependent DNA polymerase, e.g., RNA-dependent or DNA-dependent DNA polymerase. The fidelity of a DNA polymerase, including a reverse transcriptase, is measured by the error rate (the frequency of incorporating an inaccurate nucleotide, i.e., a nucleotide that is not incorporated at a template-dependent manner). The accuracy or fidelity of DNA polymerization is maintained by both the polymerase activity and the 3′-5′ exonuclease activity. The term “high fidelity” refers to an error rate equal to or lower than 33×10⁻⁶ per base pair (see Roberts J. D. et al., Science, 1988, 242: 1171-1173, the entirety hereby incorporated by reference). The fidelity or error rate of a DNA polymerase may be measured using assays known to the art (see for example, Lundburg et al., 1991 Gene, 108:1-6), and as described in Example 2 of the present specification.

A reverse transcriptase having an “increased (or enhanced or higher) fidelity” is defined as a mutant or modified reverse transcriptase (including a DNA polymerase exhibiting reverse transcriptase activity) having any increase in fidelity compared to its wild type or unmodified form, i.e., a reduction in the number of misincorporated nucleotides during synthesis of any given nucleic acid molecule of a given length. Preferably there is 1.5 to 1,000 fold (more preferably 2 to 100 fold, more preferably 3 to 10 fold) reduction in the number of misincorporated nucleotides during synthesis of any given nucleic acid molecule of a given length. For example, a mutated reverse transcriptase may misincorporate one nucleotide in the synthesis of a nucleic acid molecule segment of 1000 bases compared to an unmutated reverse transcriptase misincorporating 10 nucleotides in the same size segment. Such a mutant reverse transcriptase would be said to have an increase of fidelity of 10 fold.

An enzyme with “reduced” RNase H activity is meant that the enzyme has less than 50%, e.g., less than 40%, 30%, or less than 25%, 20%, more preferably less than 15%, less than 10%, or less than 7.5%, and most preferably less than 5% or less than 2%, of the RNase H activity of the corresponding wild type enzyme containing RNase H activity. The enzyme containing RNase activity is preferably a reverse transcriptase, such as wild type Moloney Murine Leukemia Virus (M-MLV), Avian Myeloblastosis Virus (AMV) or Rous Sarcoma Virus (RSV) reverse transcriptases and other reverse transcriptases known in the art (such as described in U.S. patent application 2003/0198944, the entirety is hereby incorporated by reference). The RNase H activity of an enzyme may be determined by a variety of assays, such as those described, for example, in U.S. Pat. Nos. 5,405,776; 6,063,608; 5,244,797; and 5,668,005 in Kotewicz, M. L., et al., Nucl. Acids Res. 16:265 (1988) and Gerard, G. F., et al., FOCUS 14(5):91 (1992), the disclosures of all of which are fully incorporated herein by reference.

As used herein, an “amplified product” refers to the single- or double-strand polynucleotide population at the end of an amplification reaction. The amplified product contains the original polynucleotide template and polynucleotide synthesized by DNA polymerase using the polynucleotide template during the amplification reaction. An amplified product preferably is produced by a reverse transcriptase and/or a DNA polymerase.

As used herein, “polynucleotide template” or “target polynucleotide template” refers to a polynucleotide (RNA or DNA) which serves as a template for a DNA polymerase to synthesize DNA in a template-dependent manner. The “amplified region,” as used herein, is a region of a polynucleotide that is to be either synthesized by reverse transcription or amplified by polymerase chain reaction (PCR). For example, an amplified region of a polynucleotide template may reside between two sequences to which two PCR primers are complementary to.

As used herein, the term “template dependent manner” refers to a process that involves the template dependent extension of a primer molecule (e.g., DNA synthesis by DNA polymerase). “Template dependent manner” refers to polynucleotide synthesis of RNA or DNA wherein the sequence of the newly synthesized strand of polynucleotide is dictated by the well-known rules of complementary base pairing (see, for example, Watson, J. D. et al., In: Molecular Biology of the Gene, 4th Ed., W. A. Benjamin, Inc., Menlo Park, Calif. (1987)).

As used herein, the term “thermostable DNA polymerase” refers to a DNA polymerase that is stable to heat, i.e., does not become irreversibly denatured (inactivated) when subjected to the elevated temperatures for the time necessary to effect denaturation of double-stranded nucleic acids. The heating conditions necessary for nucleic acid denaturation are well known in the art. As used herein, a thermostable polymerase is suitable for use in a temperature cycling reaction such as the polymerase chain reaction (“PCR”) amplification methods described in U.S. Pat. No. 4,965,188, incorporated herein by reference. A “thermostable DNA polymerase with increased reverse transcriptase activity,” according to the invention, retains the ability to effect primer extension reactions from a RNA template in a reverse transcription reaction carried out at a temperature at least 40° C., preferably, 40° C. to 80° C., and more preferably 50° C. to 70° C.

As used herein, “nucleotide” refers to a base-sugar-phosphate combination. Nucleotides are monomeric units of a nucleic acid sequence (DNA and RNA) and deoxyribonucleotides are “incorporated” into DNA by DNA polymerases. The term nucleotide includes, but is not limited to, deoxyribonucleoside triphosphates such as dATP, dCTP, dITP, dUTP, dGTP, dTTP, or derivatives thereof. Such derivatives include, for example, [aS]dATP, 7-deaza-dGTP, 7-deaza-dATP, amino-allyl dNTPs, fluorescent labeled dNTPs including Cy3, Cy5 labeled dNTPs. The term nucleotide as used herein also refers to dideoxyribonucleoside triphosphates (ddNTPs and acyclic nucleotides) and their derivatives (e.g., as described in Martinez et al., 1999, Nucl. Acids Res. 27: 1271-1274, hereby incorporated by reference in its entirety).

As used herein, a “primer” refers to a sequence of deoxyribonucleotides or ribonucleotides comprising at least 3 nucleotides. Generally, the primer comprises from about 3 to about 100 nucleotides, preferably from about 5 to about 50 nucleotides and even more preferably from about 5 to about 25 nucleotides. A primer having less than 50 nucleotides may also be referred to herein as an “oligonucleotide primer”. The primers of the present invention may be synthetically produced by, for example, the stepwise addition of nucleotides or may be fragments, parts, portions or extension products of other nucleotide acid molecules. The term “primer” is used in its most general sense to include any length of nucleotides which, when used for amplification purposes, can provide a free 3′ hydroxyl group for the initiation of DNA synthesis by a DNA polymerase, either using a RNA or a DNA template. DNA synthesis results in the extension of the primer to produce a primer extension product complementary to the nucleic acid strand to which the primer has hybridized.

“Complementary” refers to the broad concept of sequence complementarity between regions of two polynucleotide strands or between two nucleotides through base-pairing. It is known that an adenine nucleotide is capable of forming specific hydrogen bonds (“base pairing”) with a nucleotide which is thymine or uracil. Similarly, it is known that a cytosine nucleotide is capable of base pairing with a guanine nucleotide.

As used herein, the term “homology” refers to the optimal alignment of sequences (either nucleotides or amino acids), which may be conducted by computerized implementations of algorithms. “Homology”, with regard to polynucleotides, for example, may be determined by analysis with BLASTN version 2.0 using the default parameters. “Homology”, with respect to polypeptides (i.e., amino acids), may be determined using a program, such as BLASTP version 2.2.2 with the default parameters, which aligns the polypeptides or fragments being compared and determines the extent of amino acid identity or similarity between them. It will be appreciated that amino acid “homology” includes conservative substitutions, i.e. those that substitute a given amino acid in a polypeptide by another amino acid of similar characteristics. Typically seen as conservative substitutions are the following replacements: replacements of an aliphatic amino acid such as Ala, Val, Leu and Ile with another aliphatic amino acid; replacement of a Ser with a Thr or vice versa; replacement of an acidic residue such as Asp or Glu with another acidic residue; replacement of a residue bearing an amide group, such as Asn or Gln, with another residue bearing an amide group; exchange of a basic residue such as Lys or Arg with another basic residue; and replacement of an aromatic residue such as Phe or Tyr with another aromatic residue. A polypeptide sequence (i.e., amino acid sequence) or a polynucleotide sequence comprising at least 50% homology to another amino acid sequence (e.g., SEQ. ID NO:1) or another nucleotide sequence (e.g., a polynucleotide SEQ. ID NO:2 encoding SEQ ID NO.1) respectively has a homology of 50% or greater than 50%, e.g., 60%, 70%, 80%, 90% or 100% (i.e., identical).

The term “wild-type” refers to a gene or gene product which has the characteristics of that gene or gene product when isolated from a naturally occurring source. In contrast, the term “modified” or “mutant” refers to a gene or gene product which displays altered nucleotide or amino acid sequence(s) (i.e., mutations) when compared to the wild-type gene or gene product. For example, a mutant enzyme in the present invention is a mutant DNA polymerase which exhibits an increased reverse transcriptase activity, compared to its wild-type form.

As used herein, the term “mutation” refers to a change in nucleotide or amino acid sequence within a gene or a gene product, or outside the gene in a regulatory sequence compared to wild type. The change may be a deletion, substitution, point mutation, mutation of multiple nucleotides or amino acids, transposition, inversion, frame shift, nonsense mutation or other forms of aberration that differentiate the polynucleotide or protein sequence from that of a wild-type sequence of a gene or a gene product.

As used herein, the term “RT-PCR accessory factor” and “PCR accessory factor” are used interchangeably and refers to a polypeptide factor that enhances the reverse transcriptase or polymerase activity of an enzyme. The accessory factor can enhance the fidelity and/or processivity of the DNA polymerase or reverse transcriptase activity of the enzyme. Non-limiting examples of PCR accessory factors include DMSO, formamide, trehalose, nucleo capsid protein, Replication protein A, ssb, PCNA/β subunit of E. coli DNA polymerase III and θ subunit of E. coli DNA polymerase III, PEG, Glycogen, and those provided in WO 01/09347, U.S. Pat. Nos. 6,333,158 and 6,183,997, as well as Hogrefe et al., 1997, Strategies 10::93-96, which are incorporated herein by reference in their entirety.

As used herein, the term “vector” refers to a polynucleotide used for introducing exogenous or endogenous polynucleotide into host cells. A vector comprises a nucleotide sequence which may encode one or more polypeptide molecules. Plasmids, cosmids, viruses and bacteriophages, in a natural state or which have undergone recombinant engineering, are non-limiting examples of commonly used vectors to provide recombinant vectors comprising at least one desired isolated polynucleotide molecule.

As used herein, the term “transformation” or the term “transfection” refers to a variety of art-recognized techniques for introducing exogenous polynucleotide (e.g., DNA) into a cell. A cell is “transformed” or “transfected” when exogenous DNA has been introduced inside the cell membrane. The terms “transformation” and “transfection” and terms derived from each are used interchangeably.

As used herein, an “expression vector” refers to a recombinant expression cassette which has a polynucleotide which encodes a polypeptide (i.e., a protein) that can be transcribed and translated by a cell. The expression vector can be a plasmid, virus, or polynucleotide fragment.

As used herein, “isolated” or “purified” when used in reference to a polynucleotide or a polypeptide means that a naturally occurring nucleotide or amino acid sequence has been removed from its normal cellular environment or is synthesized in a non-natural environment (e.g., artificially synthesized). Thus, an “isolated” or “purified” sequence may be in a cell-free solution or placed in a different cellular environment. The term “purified” does not imply that the nucleotide or amino acid sequence is the only polynucleotide or polypeptide present, but that it is essentially free (about 90-95%, up to 99-100% pure) of non-polynucleotide or polypeptide material naturally associated with it. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein the term “encoding” refers to the inherent property of specific sequences of nucleotides in a polynucleotide, such as a gene in a chromosome or an MRNA, to serve as templates for synthesis of other polymers and macromolecules in biological processes having a defined sequence of nucleotides (i.e., rRNA, tRNA, other RNA molecules) or amino acids and the biological properties resulting therefrom. Thus a gene encodes a protein, if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. A polynucleotide that encodes a protein includes any polynucleotides that have different nucleotide sequences but encode the same amino acid sequence of the protein due to the degeneracy of the genetic code.

As used herein, the term “polymerase chain reaction” (“PCR”) refers to the method of K. B. Mullis, e.g., as described in U.S. Pat. Nos. 4,683,195 4,683,202, and 4,965,188 (each hereby incorporated in its entirety by reference) and any other improved method known in the art. PCR is a method for increasing the concentration of a segment of a target sequence in a mixture of genomic DNA without cloning or purification. This process for amplifying the target sequence typically consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i. e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

As used herein, the term “RT-PCR” refers to the replication and amplification of RNA sequences. In this method, reverse transcription is coupled to PCR, e.g., as described in U.S. Pat. No. 5,322,770, herein incorporated by reference in its entirety. In RT-PCR, the RNA template is converted to cDNA due to the reverse transcriptase activity of an enzyme, and then amplified using the polymerizing activity of the same or a different enzyme. Both thermostable and thermolabile reverse transcriptase and polymerase can be used.

Amino acid residues identified herein are preferred in the natural L-configuration. In keeping with standard polypeptide nomenclature, J. Biol. Chem., 243:3557-3559, 1969, abbreviations for amino acid residues are as shown in the following Table I.

TABLE I

| 1-Letter | 3-Letter | AMINO ACID |
|---|---|---|
| Y | Tyr | L-tyrosine |
| G | Gly | glycine |
| F | Phe | L-phenylalanine |
| M | Met | L-methionine |
| A | Ala | L-alanine |
| S | Ser | L-serine |
| I | Ile | L-isoleucine |
| L | Leu | L-leucine |
| T | Thr | L-threonine |
| V | Val | L-valine |
| P | Pro | L-proline |
| K | Lys | L-lysine |
| H | His | L-histidine |
| Q | Gln | L-glutamine |
| E | Glu | L-glutamic acid |
| W | Trp | L-tryptophan |
| R | Arg | L-arginine |
| D | Asp | L-aspartic acid |
| N | Asn | L-asparagine |
| C | Cys | L-cysteine |

### Useful Enzymes for the Invention

The present invention provides a composition containing a first enzyme exhibiting a reverse transcriptase activity and a second enzyme exhibiting a 3′-5′ exonuclease activity.

Enzymes Containing Reverse Transcriptase Activity—the First Enzyme.

Enzymes for use in the compositions, methods and kits of the present invention include any enzyme having reverse transcriptase activity. Such enzymes include, but are not limited to, retroviral reverse transcriptase, retrotransposon reverse transcriptase, hepatitis B reverse transcriptase, cauliflower mosaic virus reverse transcriptase, bacterial reverse transcriptase, E. coli DNA polymerase and klenow fragment, Tth DNA polymerase, Taq DNA polymerase (Saiki, R. K., et al., Science 239:487-491 (1988); U.S. Pat. Nos. 4,889,818 and 4,965,188), Tne DNA polymerase (WO 96/10640), Tma DNA polymerase (U.S. Pat. No. 5,374,553), C. Therm DNA polymerase from Carboxydothermus hydrogenoformans (EP0921196A1, Roche, Pleasanton, Calif., Cat. No. 2016338), ThermoScript (Invitrogen, Carsbad, Calif. Cat. No. 11731-015) and mutants, fragments, variants or derivatives thereof. As will be understood by one of ordinary skill in the art, modified reverse transcriptases may be obtained by recombinant or genetic engineering techniques that are routine and well-known in the art. Mutant reverse transcriptases can, for example, be obtained by mutating the gene or genes encoding the reverse transcriptase of interest by site-directed or random mutagenesis. Such mutations may include point mutations, deletion mutations and insertional mutations. Preferably, one or more point mutations (e.g., substitution of one or more amino acids with one or more different amino acids) are used to construct mutant reverse transcriptases of the invention. Fragments of reverse transcriptases may be obtained by deletion mutation by recombinant techniques that are routine and well-known in the art, or by enzymatic digestion of the reverse transcriptase(s) of interest using any of a number of well-known proteolytic enzymes. Mutant DNA polymerase containing reverse transcriptase activity can also be used as described in U.S. patent application Ser. No. 10/435,766, incorporated by reference in its entirety.

Polypeptides having reverse transcriptase activity that may be advantageously used in the present methods include, but are not limited to, Moloney Murine Leukemia Virus (M-MLV) reverse transcriptase, Rous Sarcoma Virus (RSV) reverse transcriptase, Avian Myeloblastosis Virus (AMV) reverse transcriptase, Rous-Associated Virus (RAV) reverse transcriptase, Myeloblastosis Associated Virus (MAV) reverse transcriptase, Human Immunodeficiency Virus (HIV) reverse transcriptase, Avian Sarcoma-Leukosis Virus (ASLV) reverse transcriptase, retroviral reverse transcriptase, retrotransposon reverse transcriptase, hepatitis B reverse transcriptase, cauliflower mosaic virus reverse transcriptase, bacterial reverse transcriptase, Thermus thermophilus (Tth) DNA polymerase, Thermus aquaticus (Taq) DNA polymerase, Thermotoga neopolitana (Tne) DNA polymerase, Thermotoga maritima (Tma) DNA polymerase, Thermococcus litoralis (Tli or VENT^{RTM}) DNA polymerase, Pyrococcus furiosus (Pfu) DNA polymerase, DEEPVENT™. Pyrococcus species GB-D DNA polymerase, Pyrococcus woosii (Pwo) DNA polymerase, Bacillus sterothermophilus (Bst) DNA polymerase, Bacillus caldophilus (Bca) DNA polymerase, Sulfoloblus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME™) DNA polymerase, Methanobacterium thermoautotrophicum (Mth) DNA polymerase, and mutants, variants and derivatives thereof.

In a preferred embodiment, an M-MLV or an AMV reverse transcriptase is used.

Particularly preferred for use in the invention are the variants of these enzymes that are reduced in RNase H activity (i.e., RNase H-enzymes). Preferably, the enzyme has less than 20%, more preferably less than 15%, 10% or 5%, and most preferably less than 2%, of the RNase H activity of a wildtype or “RNase H⁺” enzyme such as wildtype M-MLV or AMV reverse transcriptases. The RNase H activity of any enzyme may be determined by a variety of assays, such as those described, for example, in U.S. Pat. Nos. 5,244,797; 5,405,776; 5,668,005; and 6,063,608; in Kotewicz, M. L., et al., Nucl. Acids Res. 16:265 (1988) and in Gerard, G. F., et al., FOCUS 14(5):91 (1992), the disclosures of all of which are filly incorporated herein by reference.

Particularly preferred RNase H-reverse transcriptase enzymes for use in the invention include, but are not limited to, M-MLV H-reverse transcriptase, RSV H-reverse transcriptase, AMV H-reverse transcriptase, RAV H-reverse transcriptase, MAV H-reverse transcriptase and HIV H-reverse transcriptase for example as previously described (see U.S. Pat. Nos. 5,244,797; 5,405,776; 5,668,005 and 6,063,608; and WO 98/47912, the entirety of each is incorporated by reference). The RNase H activity of any enzyme may be determined by a variety of assays, such as those described, for example, in U.S. Pat. Nos. 5,244,797; 5,405,776; 5,668,005 and 6,063,608; in Kotewicz, M. L., et al., Nucl. Acids Res. 16:265 (1988); and in Gerard, G. F., et al., FOCUS 14(5):91 (1992), the disclosures of all of which are fully incorporated herein by reference. It will be understood by one of ordinary skill, however, that any enzyme capable of producing a DNA molecule from a ribonucleic acid molecule (i.e., having reverse transcriptase activity) that is substantially reduced in RNase H activity may be equivalently used in the compositions, methods and kits of the invention.

Polypeptides having reverse transcriptase activity for use in the invention may be obtained commercially, for example, from Invitrogen, Inc. (Carlsbad, Calif.), Pharmacia (Piscataway, N.J.), Sigma (Saint Louis, Mo.) or Roche Molecular System (Pleasanton, Calif.). Alternatively, polypeptides having reverse transcriptase activity may be isolated from their natural viral or bacterial sources according to standard procedures for isolating and purifying natural proteins that are well-known to one of ordinary skill in the art (see, e.g., Houts, G. E., et al., J. Virol. 29:517 (1979)). In addition, the polypeptides having reverse transcriptase activity may be prepared by recombinant DNA techniques that are familiar to one of ordinary skill in the art (see, e.g., Kotewicz, M. L., et al., Nucl. Acids Res. 16:265 (1988); Soltis, D. A., and Skalka, A. M., Proc. Natl. Acad. Sci. USA 85:3372-3376 (1988)). The entire teaching of the above references is hereby incorporated by reference.

Enzymes that are reduced in RNase H activity may be obtained by methods known in the art, e.g., by mutating the RNase H domain within the reverse transcriptase of interest, preferably by one or more point mutations, one or more deletion mutations, and/or one or more insertion mutations as described above, e.g., as described in U.S. Pat. No. 6,063,608 hereby incorporated in its entirety by reference.

In a preferred embodiment of the present invention, a M-MLV reverse transcriptase with reduced RNase H activity or a AMV reverse transcriptase with reduced RNase H activity was used.

Two or more enzymes with reverse transcriptase activity may be used in a single composition, e.g., the same reaction mixture. Enzymes used in this fashion may have distinct reverse transcription pause sites with respect to the template nucleic acid, as described in U.S. patent application 2003/0198944A1, hereby incorporated in its entirety by reference.

The enzyme containing reverse transcriptase activity of the present invention may also include a mutant or modified reverse transcriptase where one or more amino acid changes have been made which renders the enzyme more faithful (higher fidelity) in nucleic acid synthesis, e.g., as described in U.S. patent application 2003/0003452A1, hereby incorporated in its entirety by reference.

Enzymes Containing 3′-5′ Exonuclease Activity—the Second Enzyme.

The second enzyme comprising 3′-5′ exonuclease activity (i.e., proofreading DNA polymerase or an autonomous exonuclease) according to the invention includes, but is not limited to, DNA polymerases, E. coli exonuclease I, E. coli exonuclease III, E. coli recBCD nuclease, mung bean nuclease, and the like (see for example, Kuo, 1994, Ann N Y Acad Sci., 726:223-34, Shevelev IV, Hubscher U., 2002, Nat Rev Mol Cell Biol. 3(5):364-76).

Any proofreading DNA polymerase could be used as the second enzyme of the present invention. Examples can be found in many DNA polymerase families including, but are not limited to, family A DNA polymerases (e.g., T7 DNA polymerase), family C DNA polymerases, family B DNA polymerases (e.g., including Bacteriophage T4 DNA polymerase, φ29 DNA polymerase; E. coli pol II DNA polymerase; human DNA polymerase δ, human DNA polymerase γ, archaeal DNA polymerase (as described in U.S. patent application 2003/0143577, hereby incorporated by reference in its entirety), Eubacterial Family A DNA polymerases (with proofreading activity, such as Thermotoga maritima (UlTma fragment)); family D DNA polymerases (unrelated to Families A, B, C) etc. A DNA polymerase with reduced DNA polymerization activity but containing 3′-5′ exonuclease activity, e.g., as described in U.S. patent application 2003/0143577 (incorporated in its entirety by reference) can be also used.

Enzymes possessing 3′-5′ exonuclease activity for use in the present compositions and methods may be isolated from natural sources or produced through recombinant DNA techniques. Preferably, the enzyme comprising 3′-5′ exonuclease activity is a DNA polymerase.

More preferably, the second enzyme containing 3′-5′ exonuclease activity is a non-alpha type DNA polymerase.

Still more preferably, the second enzyme containing 3′-5′ exonuclease activity is a family A or family C DNA polymerase, e.g., as listed in Ito. et al., Nucleic Acids Research (1991), 19: 4045-4057, the entirety of which is incorporated by reference.

Classification of DNA polymerases

| | | | | References |
|---|---|---|---|---|
| A. Family A DNA polymerases | | | | |
| | 1. Bacterial DNA polymerases | | | |
| | | a) | E. coli DNA polymerase I | Joyce, C.M. et al., |
| | | | | (1982), J. Biol. Chem., |
| | | | | 257: 1958-1964. |
| | | b) | Streptococcus pneumoniae | Lopez, P. et al., |
| | | | DNA polymerase I | (1989), J. Biol. Chem., |
| | | | | 264: 4255-4263. |
| | | c) | Thermus aquaticus DNA | Lawyer, F. C. et al., |
| | | | polymerase I | (1989), J. Biol. Chem., |
| | | | | 264: 6427-6437. |
| | 2. Bacteriophage DNA polymerases | | | |
| | | a) | T5 DNA polymerase | Leavitt, M. C. et al., |
| | | | | (1989), Proc. Natl. |
| | | | | Acad. Sci. U.S.A., |
| | | | | 86: 4465-4469. |
| | | b) | T7 DNA polymerase | Dunn, J. J. et al., |
| | | | | (1983), J. Mol. Biol., |
| | | | | 166: 477-535. |
| | | c) | Spo2 DNA polymerase | Rådén, B., et al., |
| | | | | (1984), J. Virol., |
| | | | | 52: 9-15. |
| | 3. Mitochondrial DNA polymerase | | | |
| | | | Yeast mitochondrial DNA | Foury, F., (1989), |
| | | | polyermerase (MIP1) | J. Biol. Chem., |
| | | | | 264: 20552-20560. |
| B. Family C DNA polymerases | | | | |
| | | Bacterial replicative DNA polymerases | | |
| | | a) | E. coli DNA polymerase III | Tomasiewicz, H. G. et |
| | | | or subunit | al., (1987), J. |
| | | | | Bacteriol, 169: |
| | | | | 5735-5744. |
| | | b) | Salmonella typhimurium DNA | Lancy, E. D. et al., |
| | | | polymerase III or subunit | (1989), J. Bacteriol., |
| | | | | 171: 5581-5586. |
| | | c) | Bacillus subtilis DNA poly- | Hammond, R. A. et al., |
| | | | merase III | (1991), Gene, 98: |
| C. Family X DNA polymerases | | | | 29-36. |
| | | a) | Rat DNA polymerase β | Matsukage, A. et al., |
| | | | | (1987), J. Biol. Chem., |
| | | | | 262: 8960-8962. |
| | | b) | Human DNA polymerase β | 1) Abbotts, J. et al., |
| | | | | (1988), Biochemistry, |
| | | | | 27: 901-909. |
| | | | | 2) SenGupta, D. N. |
| | | | | et al., (1986), Biochem. |
| | | | | Biophys. Res. Comm., |
| | | | | 136: 341-347. |
| | | c) | Human terminal deoxynucleo- | Peterson, R. C. et al., |
| | | | tidyltransferase (TdT) | (1985), J. Biol. Chem., |
| | | | | 260: 10495-19502. |
| | | d) | Bovine terminal deoxynucleo- | Koiwai, O. et al., |
| | | | tidyltransferase (TdT) | (1986), Nucl. Acids |
| | | | | Res., 14: 5777-5792. |
| | | e) | Mouse terminal deoxynucleo- | Koiwai, O. et al., |
| | | | tidyltransferase (TdT) | (1986), Nucl. Acids |
| | | | | Res., 14: 5777-5792. |

The second enzyme containing 3′-5′ exonuclease activity may be thermostable or non-thermo stable.

A thermostable second enzyme can be any enzyme exhibiting 3′5′ exonuclease activity known in the art such as those described above. A thermostable second enzyme can also be, e.g., the dnaQ gene product of T. thermophilus, as described in U.S. Pat. No. 6,238,905, hereby incorporated in its entirety by reference.

In preferred embodiments of the invention, the second enzyme exhibiting 3′-5′ exonuclease activity is a non-thermostable DNA polymerase.

In one embodiment, the second enzyme containing 3′-5′ exonuclease activity is P53 protein, or φ29 DNA polymerase.

In another embodiment, the second enzyme containing 3′-5′ exonuclease activity is a family A or family C DNA polymerase.

In another embodiment of the invention, the second enzyme exhibiting 3′-5′ exonuclease activity is E. coli DNA polymerase III, e.g., as described in Perrino et al. (supra, hereby incorporated by reference in its entirety).

Preferably, the second enzyme exhibiting 3′-5′ exonuclease activity is the epsilon (ε) subunit of E. coli DNA polymerase III.

In one embodiment of the present invention, the second enzyme exhibiting 3′-5′ exonuclease activity contains an amino acid sequence of SEQ. ID NO:1.

3′-5′ exonuclease activity can be measured by any known methods in the art. In one embodiment, unit activity of a 3′-5′ exonuclease (e.g., the epsilon subunit of E. coli DNA polymerase III) is determined (e.g., as described in Hamdan, S. et al., Biochemistry 2002, 41:5266-5275) spectrophotometrically by monitoring the production of p-nitrophenolate anion produced by hydrolysis of pNP-TMP at 420 nm. A stock solution of pNP-TMP is diluted with assay buffer [50 mM Tris.HCl (pH 8), 150 mM NaCl, and 1 mM DTT, to 970-980 μl] to a final concentration of 3 mM. Following equilibration at 25° C., solutions of MnCl₂ (10 μl) and enzyme (10-20 μl) are added to give final concentrations of 1 mM and 100-400 nM, respectively. Changes in A₄₂₀ are followed over a 90 seconds. Rates of pNP-TMP hydrolysis are calculated using a value of 12950 M⁻cm⁻¹ for the ε420 of p-nitrophenol at pH 8.

Formulation of Enzyme Blend

The present invention provides a composition containing the first and the second enzymes as described above. The first and second enzymes may be provided separately and then mixed in a reaction mixture or they may be provided as a mixed blend prior to use in a reaction. To form the preferred compositions of the present invention, the first and the second enzymes are preferably admixed in a buffered salt solution. One or more DNA polymerases and/or one or more nucleotides may optionally be added to make the compositions of the invention. More preferably, the enzymes are provided at working concentrations in buffered salt solutions.

The water used in forming the compositions of the present invention is preferably distilled, deionized and sterile filtered (through a 0. 1-0.2 micrometer filter), and is free of contamination by DNase and RNase enzymes. Such water is available commercially, for example from Sigma Chemical Company (Saint Louis, Mo.), or may be made as needed according to methods well known to those skilled in the art.

Two or more enzymes containing reverse transcriptase activity and/or two or more enzymes containing 3′-5′ exonuclease activity may be included in the compositions of the present invention. In addition to the enzyme components, the present compositions preferably comprise one or more buffers and other components necessary for synthesis of a nucleic acid molecule. Particularly preferred buffers for use in forming the present compositions are the acetate, sulfate, hydrochloride, phosphate or free acid forms of Tris-(hydroxymethyl)aminomethane (TRIS^{RTM}), although alternative buffers of the same approximate ionic strength and pKa as TRIS^{RTM} may be used with equivalent results. In addition to the buffer salts, cofactor salts such as those of potassium (preferably potassium chloride or potassium acetate) and magnesium (preferably magnesium chloride or magnesium acetate) are included in the compositions. Addition of one or more carbohydrates and/or sugars to the compositions and/or synthesis reaction mixtures may also be advantageous, to support enhanced stability of the compositions and/or reaction mixtures upon storage. Preferred such carbohydrates or sugars for inclusion in the compositions and/or synthesis reaction mixtures of the invention include, but are not limited to, sucrose, trehalose, and the like. Furthermore, such carbohydrates and/or sugars may be added to the storage buffers for the enzymes used in the production of the enzyme compositions and kits of the invention. Such carbohydrates and/or sugars are commercially available from a number of sources, including Sigma (St. Louis, Mo.).

It is often preferable to first dissolve the buffer salts, cofactor salts and carbohydrates or sugars at working concentrations in water and to adjust the pH of the solution prior to addition of the enzymes. In this way, the pH-sensitive enzymes will be less subject to acid- or alkaline-mediated inactivation during formulation of the present compositions.

To formulate the buffered salts solution, a buffer salt which is preferably a salt of Tris(hydroxymethyl)aminomethane (TRIS^{RTM}), and most preferably the hydrochloride salt thereof, is combined with a sufficient quantity of water to yield a solution having a TRIS^{RTM} concentration of 5-150 millimolar, preferably 10-60 millimolar, and most preferably about 20-60 millimolar. To this solution, a salt of magnesium (preferably either the chloride or acetate salt thereof) may be added to provide a working concentration thereof of 1-10 millimolar, preferably 1.5-8.0 millimolar, and most preferably about 3-7.5 millimolar. A salt of potassium (preferably a chloride or acetate salt of potassium) may also be added to the solution, at a working concentration of 10-100 millimolar and most preferably about 75 millimolar. A reducing agent such as dithiothreitol may be added to the solution, preferably at a final concentration of about 1-100 mM, more preferably a concentration of about 5-50 mM or about 7.5-20 mM, and most preferably at a concentration of about 10 mM. Preferred concentrations of carbohydrates and/or sugars for inclusion in the compositions of the invention range from about 5% (w/v) to about 30% (w/v), about 7.5% (w/v) to about 25% (w/v), about 10% (w/v) to about 25% (w/v), about 10% (w/v) to about 20% (w/v), and preferably about 10% (w/v) to about 15% (w/v). A small amount of a salt of ethylenediaminetetraacetate (EDTA), such as disodium EDTA, may also be added (preferably about 0.1 millimolar), although inclusion of EDTA does not appear to be essential to the function or stability of the compositions of the present invention. After addition of all buffers and salts, this buffered salt solution is mixed well until all salts are dissolved, and the pH is adjusted using methods known in the art to a pH value of 7.4 to 9.2, preferably 8.0 to 9.0, and most preferably about 8.4.

To these buffered salt solutions, the enzymes (reverse transcriptase and/or DNA polymerase) are added to produce the compositions of the present invention. In a preferred embodiment, M-MLV RT or AMV is preferably added at a working concentration in the solution of 500 to 50,000 units per milliliter, 500 to 30,000 units per milliliter, 500 to 25,000 units per milliliter, 500 to 22,500 units per milliliter, 500 to 20,000 units per milliliter. In one preferred embodiment, the M-MLV RT with reduced RNase H activity is added at a working concentration of 1250 unit per milliliter (25 unit per 20 μl reaction). In another preferred embodiment, the AMV RT is added at a working concentration of 500 unit per milliliter (10 unit per 20 μl reaction).

The ratio of the first enzyme to the second enzyme in the subject composition may vary according to the present invention. Preferably, for a 20 μl reaction, the composition results in a working amount of 0.1-500 units of reverse transcriptase activity from the first enzyme (e.g., a reverse transcriptase or a DNA polymerase with reverse transcription activity), more preferably, 5-100 units of reverse transcriptase activity from the first enzyme, more preferably, 10-50 units of reverse transcriptase activity from the first enzyme, more preferably, 20-40 units of reverse transcriptase activity from the first enzyme. Preferably, for a 20 μl reaction, the composition results in a working amount of 0.001-50 units of 3′-5′ exonuclease activity from the second enzyme, more preferably, 0.01-25 units of 3′-5′ exonuclease activity from the second enzyme, more preferably, 0.01-10 units of 3′-5′ exonuclease activity from the second enzyme. The ratio of the reverse transcriptase activity (in units) over the 3′-5′ exonuclease activity (in units) ranges from 5000 to 1, preferably, between 1500-5, more preferably between 100-10.

In a preferred embodiment, the second enzyme containing 3′-5′ exonuclease activity is a DNA polymerase. The DNA polymerase can be either thermostable or non-thermostable. The enzymes may be added to the solution in any order, or may be added simultaneously.

In another preferred embodiment, the second enzyme containing 3′-5′ exonuclease activity is an autonomous exonuclease as described in Igor V. Shevelev & Ulrich Hübscher (2002, supra). Such exonuclease may be thermostable or non-thermostable.

A thermostable 3′-5′ exonuclease may be one from archaea or a high temperature eubacteria. A non-thermostable 3′-5′ exonuclease may be one from mammalian or eubactria, for example, exonuclease III, E. coli epsilon subunit, P53, etc.

Preferably, the non-thermostable second enzyme is a polypeptide having at least 50% homology to SEQ. ID NO:1. More preferably, the non-thermostable second enzyme is the epsilon subunit of E. coli DNA polymerase III. Preferably, for a 20 μl reaction, the epsilon subunit of E. coli DNA polymerase III is used at a working amount of 0.017 u to 3.4 u (5 ng to 1000 ng per 20 μl reaction), more preferably 0.1 u to 1 u.

The compositions of the invention may further comprise one or more nucleotides, which are preferably deoxynucleoside triphosphates (dNTPs) or dideoxynucleoside triphosphates (ddNTPs). The dNTP components of the present compositions serve as the “building blocks” for newly synthesized nucleic acids, being incorporated therein by the action of the polymerases, and the ddNTPs may be used in sequencing methods according to the invention. Examples of nucleotides suitable for use in the present compositions include, but are not limited to, dUTP, dATP, dTTP, dCTP, dGTP, dITP, 7-deaza-dGTP, α-thio-dATP, α-thio-dTTP, α-thio-dGTP, α-thio-dCTP, ddUTP, ddATP, ddTTP, ddCTP, ddGTP, ddITP, 7-deaza-ddGTP, α-thio-ddATP, α-thio-ddTTP, α-thio-ddGTP, α-thio-ddCTP, amino allyl modified nucleotides such as amino allyl dUTP, amino allyl UTP or amino allyl dCTP, fluorescent labeled nucleotides such as Cy5 or Cy3 labeled dNTPs, or derivatives thereof, all of which are available commercially from sources including New England BioLabs (Beverly, Mass.) and Sigma Chemical Company (Saint Louis, Mo.).

“Amino allyl modified nucleotide” refers to a nucleotide that has been modified to contain a primary amine at the 5′-end of the nucleotide, preferably with one or more methylene groups disposed between the primary amine and the nucleic acid portion of the nucleic acid polymer. Six is a preferred number of methylene groups. Amino allyl modified nucleotides can be introduced into nucleic acids by polymerases disclosed herein, with amino allyl dUTP or amino allyl dCTP.

The nucleotides may be unlabeled, or they may be detectably labeled by coupling them by methods known in the art with radioisotopes (e.g.,³H, ¹⁴C, ³²p or ³⁵S), vitamins (e.g., biotin), fluorescent moieties (e.g., fluorescein, rhodamine, Texas Red, or phycoerythrin, Cy3, Cy5), chemiluminescent labels (e.g., using the PHOTO-GENE™ or ACES™ chemiluminescence systems, available commercially from Invitrogen, Inc., Carlsbad, Calif.), dioxigenin and the like. Labeled nucleotides may also be obtained commercially, for example from Invitrogen, Inc. (Carlsbad, Calif.) or Sigma Chemical Company (Saint Louis, Mo.). In the present compositions, the nucleotides are added to give a working concentration of each nucleotide of 10-4000 micromolar, 50-2000 micromolar, 100-1500 micromolar, or 200-1200 micromolar, and most preferably a concentration of 1000 micromolar.

The compositions of the present invention may also include PCR accessory factors and other additives that facilitate reverse transcription or amplification.

PCR enhancing factors may also be used to improve efficiency of the amplification. For example, one PCR accessory factor is PEF as described in U.S. Pat. No. 6,183,997, hereby incorporated in its entirety by reference. PEF comprises either P45 in native form (as a complex of P50 and P45) or as a recombinant protein. In the native complex of Pfu P50 and P45, only P45 exhibits PCR enhancing activity. The P50 protein is similar in structure to a bacterial flavoprotein. The P45 protein is similar in structure to dCTP deaminase and dUTPase, but it functions only as a dUTPase converting dUTP to dUMP and pyrophosphate. PEF, according to the present invention, can also be selected from the group consisting of: an isolated or purified naturally occurring polymerase enhancing protein obtained from an archeabacteria source (e.g., Pyrococcus furiosus); a wholly or partially synthetic protein having the same amino acid sequence as Pfu P45, or analogs thereof possessing polymerase enhancing activity; polymerase-enhancing mixtures of one or more of said naturally occurring or wholly or partially synthetic proteins; polymerase-enhancing protein complexes of one or more of said naturally occurring or wholly or partially synthetic proteins; or polymerase-enhancing partially purified cell extracts containing one or more of said naturally occurring proteins (U.S. Pat. No. 6,183,997, supra). The PCR enhancing activity of PEF is defined by means well known in the art. The unit definition for PEF is based on the dUTPase activity of PEF (P45), which is determined by monitoring the production of pyrophosphate (PPi) from dUTP. For example, PEF is incubated with dUTP (10 mM dUTP in 1 x cloned Pfu PCR buffer) during which time PEF hydrolyzes dUTP to dUMP and PPi. The amount of PPi formed is quantitated using a coupled enzymatic assay system that is commercially available from Sigma (#P7275). One unit of activity is functionally defined as 4.0 nmole of PPi formed per hour (at 85° C.).

Other PCR additives may also affect the accuracy and specificity of PCR reaction. EDTA less than 0.5 mM may be present in the amplification reaction mix. Detergents such as Tween-20™ and Nonide™ P-40 are present in the enzyme dilution buffers. A final concentration of non-ionic detergent approximately 0.1% or less is appropriate, however, 0.01-0.05% is preferred and will not interfere with polymerase activity. Similarly, glycerol is often present in enzyme preparations and is generally diluted to a concentration of 1-20% in the reaction mix. Glycerol (5-10%), formamide (1-5%) or DMSO (2-10%) can be added in PCR for template DNA with high GC content or long length (e.g., >1 kb). These additives change the Tm (melting temperature) of primer-template hybridization reaction and the thermostability of polymerase enzyme. BSA (up to 0.8 μg/μl) can improve efficiency of PCR reaction. Betaine (0.5-2M) is also useful for PCR over high GC content and long fragments of DNA. Tetramethylammonium chloride (TMAC, >50 mM), Tetraethylammonium chloride (TEAC), and Trimethlamine N-oxide (TMANO) may also be used. Test PCR reactions may be performed to determine optimum concentration of each additive mentioned above.

In one embodiment, θ subunit of E. coli DNA polymerase III is used to increase the thermostability of the epsilon subunit (e.g., see Hamdan et al., 2002, Biochemistry, 41:5266-5275). The θ subunit may also be used with any other epsilon subunit according to the invention to increase the thermostability of the enzyme blend and to improve the sensitivity and fidelity of thermostable reverse transcriptases.

To reduce component deterioration, storage of the reagent compositions is preferably at about 4° C. for up to one day, or most preferably at −20° C. for up to one year.

In another aspect, the compositions of the invention may be prepared and stored in dry form in the presence of one or more carbohydrates, sugars, or synthetic polymers. Preferred carbohydrates, sugars or polymers for the preparation of dried compositions or reverse transcriptases include, but are not limited to, sucrose, trehalose, and polyvinylpyrrolidone (PVP) or combinations thereof See, e.g., U.S. Pat. Nos. 5,098,893, 4,891,319, and 5,556,771, the disclosures of which are entirely incorporated herein by reference. Such dried compositions and enzymes may be stored at various temperatures for extended times without significant deterioration of enzymes or components of the compositions of the invention. Preferably, the dried reverse transcriptases or compositions are stored at 4° C. or at −20° C.

cDNA Synthesis

In accordance with the invention, cDNA molecules (single-stranded or double-stranded) may be prepared from a variety of nucleic acid template molecules. Preferred nucleic acid molecules for use in the present invention include single-stranded or double-stranded DNA and RNA molecules, as well as double-stranded DNA:RNA hybrids. More preferred nucleic acid molecules include messenger RNA (mRNA), transfer RNA (tRNA) and ribosomal RNA (rRNA) molecules, although MRNA molecules are the preferred template according to the invention.

The present invention provides compositions and methods for high fidelity cDNA synthesis. The subject compositions and methods may also increase the efficiency and of the reverse transcription as well as the length of the cDNA synthesized. As a result, the fidelity, efficiency, and yield of subsequent manipulations of the synthesized cDNA (e.g., amplification, sequencing, cloning, etc.) are also increased. The nucleic acid molecules that are used to prepare cDNA molecules according to the methods of the present invention may be prepared synthetically according to standard organic chemical synthesis methods that will be familiar to one of ordinary skill. More preferably, the nucleic acid molecules may be obtained from natural sources, such as a variety of cells, tissues, organs or organisms. Cells that may be used as sources of nucleic acid molecules may be prokaryotic (bacterial cells, including but not limited to those of species of the genera Escherichia, Bacillus, Serratia, Salmonella, Staphylococcus, Streptococcus, Clostridium, Chlamydia, Neisseria, Treponema, Mycoplasma, Borrelia, Legionella, Pseudomonas, Mycobacterium, Helicobacter, Erwinia, Agrobacterium, Rhizobium, Xanthomonas and Streptomyces) or eukaryotic (including fungi (especially yeasts), plants, protozoans and other parasites, and animals including insects (particularly Drosophila spp. cells), nematodes (particularly Caenorhabditis elegans cells), and mammals (particularly human cells)).

Mammalian somatic cells that may be used as sources of nucleic acids include blood cells (reticulocytes and leukocytes), endothelial cells, epithelial cells, neuronal cells (from the central or peripheral nervous systems), muscle cells (including myocytes and myoblasts from skeletal, smooth or cardiac muscle), connective tissue cells (including fibroblasts, adipocytes, chondrocytes, chondroblasts, osteocytes and osteoblasts) and other stromal cells (e.g., macrophages, dendritic cells, Schwann cells). Mammalian germ cells (spermatocytes and oocytes) may also be used as sources of nucleic acids for use in the invention, as may the progenitors, precursors and stem cells that give rise to the above somatic and germ cells. Also suitable for use as nucleic acid sources are mammalian tissues or organs such as those derived from brain, kidney, liver, pancreas, blood, bone marrow, muscle, nervous, skin, genitourinary, circulatory, lymphoid, gastrointestinal and connective tissue sources, as well as those derived from a mammalian (including human) embryo or fetus.

Any of the above prokaryotic or eukaryotic cells, tissues and organs may be normal, diseased, transformed, established, progenitors, precursors, fetal or embryonic. Diseased cells may, for example, include those involved in infectious diseases (caused by bacteria, fungi or yeast, viruses (including AIDS, HIV, HTLV, herpes, hepatitis and the like) or parasites), in genetic or biochemical pathologies (e.g., cystic fibrosis, hemophilia, Alzheimer's disease, muscular dystrophy or multiple sclerosis) or in cancerous processes. Transformed or established animal cell lines may include, for example, COS cells, CHO cells, VERO cells, BHK cells, HeLa cells, HepG2 cells, K562 cells, 293 cells, L929 cells, F9 cells, and the like. Other cells, cell lines, tissues, organs and organisms suitable as sources of nucleic acids for use in the present invention will be apparent to one of ordinary skill in the art.

Once the starting cells, tissues, organs or other samples are obtained, nucleic acid molecules (such as mRNA) may be isolated therefrom by methods that are well-known in the art (See, e.g., Maniatis, T., et al., Cell 15:687-701 (1978); Okayama, H., and Berg, P., Mol. Cell. Biol. 2:161-170 (1982); Gubler, U., and Hoffman, B. J., Gene 25:263-269 (1983)). The nucleic acid molecules thus isolated may then be used to prepare cDNA molecules and cDNA libraries in accordance with the present invention.

In the practice of the invention, cDNA molecules or cDNA libraries may be produced by mixing one or more nucleic acid molecules obtained as described above, which is preferably one or more MRNA molecules such as a population of mRNA molecules, with the composition of the invention, under conditions favoring the reverse transcription of the nucleic acid molecule by the action of the enzymes of the compositions to form a cDNA molecule (single-stranded or double-stranded). Thus, the method of the invention comprises (a) mixing one or more nucleic acid templates (preferably one or more RNA or mRNA templates, such as a population of mRNA molecules) with a composition of the invention (e.g., an enzyme mixture comprising a first enzyme exhibiting a reverse transcriptase activity with reduced RNase H activity and a second enzyme exhibiting a 3′-5′ exonuclease activity) and (b) incubating the mixture under conditions sufficient to permit cDNA synthesis, e.g., to all or a portion of the one or more templates.

The compositions of the present invention may be used in conjunction with methods of CDNA synthesis such as those described in the Examples below, or others that are well-known in the art (see, e.g., Gubler, U., and Hoffman, B. J., Gene 25:263-269(1983); Krug, M. S., and Berger, S. L., Meth. Enzymol. 152:316-325 (1987); Sambrook, J., et al., Molecular Cloning: A Laboratory Manual, 2nd ed., Cold Spring Harbor, N.Y.: Cold Spring Harbor Laboratory Press, pp. 8.60-8.63 (1989)), to produce cDNA molecules or libraries.

The invention is directed to such methods which further produce a first strand and a second strand cDNA, as known in the art. According to the invention, the first and second strand cDNAs produced by the methods may form a double stranded DNA molecule which may be a full length cDNA molecule.

Other methods of cDNA synthesis which may advantageously use the present invention will be readily apparent to one of ordinary skill in the art.

Subsequent Manipulation of Synthesized cDNA

Having obtained cDNA molecules or libraries according to the present methods, these cDNAs may be isolated or the reaction mixture containing the cDNAs may be directly used for further analysis or manipulation. Detailed methodologies for purification of cDNAs are taught in the GENETRAPPER™ manual (Invitrogen, Inc. Carlsbad, Calif.), which is incorporated herein by reference in its entirety, although alternative standard techniques of cDNA isolation such as those described in the Examples below or others that are known in the art (see, e.g., Sambrook, J., et al., Molecular Cloning: A Laboratory Manual, 2nd ed., Cold Spring Harbor, N.Y.: Cold Spring Harbor Laboratory Press, pp. 8.60-8.63 (1989)) may also be used.

In other aspects of the invention, the invention may be used in methods for amplifying and sequencing nucleic acid molecules. Nucleic acid amplification methods according to this aspect of the invention may be one- step (e.g., one-step RT-PCR) or two-step (e.g., two-step RT-PCR) reactions. According to the invention, one-step RT-PCR type reactions may be accomplished in one tube thereby lowering the possibility of contamination. Such one-step reactions comprise (a) mixing a nucleic acid template (e.g., mRNA) with a composition of present invention and (b) incubating the mixture under conditions sufficient to permit amplification. Two-step RT-PCR reactions may be accomplished in two separate steps. Such a method comprises (a) mixing a nucleic acid template (e.g., mRNA) with a composition of present invention, (b) incubating the mixture under conditions sufficient to permit cDNA synthesis, (c) mixing the reaction mixture in (b) with one or more DNA polymerases and (d) incubating the mixture of step (c) under conditions sufficient to permit amplification. For amplification of long nucleic acid molecules (i.e., greater than about 3-5 Kb in length), a combination of DNA polymerases may be used, such as one DNA polymerase having 3′-5′ exonuclease activity and another DNA polymerase being reduced in 3′-5′ exonuclease activity.

The subject composition may be used for nucleic acid sequencing. Nucleic acid sequencing methods according to this aspect of the invention may comprise both cycle sequencing (sequencing in combination with amplification) and standard sequencing reactions. The sequencing method of the invention thus comprises (a) mixing a nucleic acid molecule to be sequenced with a composition of the present invention and one or more terminating agents, (b) incubating the mixture under conditions sufficient to permit cDNA synthesis and/or amplification, and (c) separating the population to determine the nucleotide sequence of the nucleic acid molecule sequenced.

Amplification methods which may be used in accordance with the present invention include PCR (e.g., U.S. Pat. Nos. 4,683,195 and 4,683,202), Strand Displacement Amplification (SDA; e.g., U.S. Pat. No. 5,455,166; EP 0 684 315), and Nucleic Acid Sequence-Based Amplification (NASBA; e.g., U.S. Pat. No. 5,409,818; EP 0 329 822). Nucleic acid sequencing techniques which may employ the present compositions include dideoxy sequencing methods such as those disclosed in U.S. Pat. Nos. 4,962,022 and 5,498,523, as well as more complex PCR-based nucleic acid fingerprinting techniques such as Random Amplified Polymorphic DNA (RAPD) analysis (Williams, J. G. K., et al., Nucl. Acids Res. 18(22):6531-6535, 1990), Arbitrarily Primed PCR (AP-PCR; Welsh, J., and McClelland, M., Nucl. Acids Res. 18(24):7213-7218, 1990), DNA Amplification Fingerprinting (DAF; Caetano-Anolles et al., Bio/Technology 9:553-557, 1991), microsatellite PCR or Directed Amplification of Minisatellite-region DNA (DAMD; Heath, D. D., et al., Nucl. Acids Res. 21(24): 5782-5785, 1993), and Amplification Fragment Length Polymorphism (AFLP) analysis (EP 0 534 858; Vos, P., et al., Nucl Acids Res. 23(21):4407-4414, 1995; Lin, J. J., and Kuo, J., FOCUS 17(2):66-70, 1995). In a particularly preferred aspects, the invention may be used in methods of amplifying or sequencing a nucleic acid molecule comprising one or more polymerase chain reactions (PCRs), such as any of the PCR-based methods described above. All references are entirely incorporated by reference.

The primer used for synthesizing a cDNA from an RNA as a template in the present invention is not limited to a specific one as long as it is an oligonucleotide that has a nucleotide sequence complementary to that of the template RNA and that can anneal to the template RNA under reaction conditions used. The primer may be an oligonucleotide such as an oligo(dT) or an oligonucleotide having a random sequence (a random primer) or a gene-specific primer.

The nucleic acid molecules (e.g., synthesized cDNA or amplified product) or cDNA libraries prepared by the methods of the present invention may be further characterized, for example by cloning and sequencing (i.e., determining the nucleotide sequence of the nucleic acid molecule), by the sequencing methods of the invention or by others that are standard in the art (see, e.g., U.S. Pat. Nos. 4,962,022 and 5,498,523, which are directed to methods of DNA sequencing). Alternatively, these nucleic acid molecules may be used for the manufacture of various materials in industrial processes, such as hybridization probes by methods that are well-known in the art. Production of hybridization probes from cDNAs will, for example, provide the ability for those in the medical field to examine a patient's cells or tissues for the presence of a particular genetic marker such as a marker of cancer, of an infectious or genetic disease, or a marker of embryonic development. Furthermore, such hybridization probes can be used to isolate DNA fragments from genomic DNA or cDNA libraries prepared from a different cell, tissue or organism for further characterization.

The nucleic acid molecules (e.g., synthesized cDNA or amplified product) of the present invention may also be used to prepare compositions for use in recombinant DNA methodologies. Accordingly, the present invention relates to recombinant vectors which comprise the cDNA or amplified nucleic acid molecules of the present invention, to host cells which are genetically engineered with the recombinant vectors, to methods for the production of a recombinant polypeptide using these vectors and host cells, and to recombinant polypeptides produced using these methods.

Recombinant vectors may be produced according to this aspect of the invention by inserting, using methods that are well-known in the art, one or more of the cDNA molecules or amplified nucleic acid molecules prepared according to the present methods into a vector. The vector used in this aspect of the invention may be, for example, a phage or a plasmid, and is preferably a plasmid. Preferred are vectors comprising cis-acting control regions to the nucleic acid encoding the polypeptide of interest. Appropriate trans-acting factors may be supplied by the host, supplied by a complementing vector or supplied by the vector itself upon introduction into the host.

In certain preferred embodiments in this regard, the vectors provide for specific expression (and are therefore termed “expression vectors”), which may be inducible and/or cell type-specific. Particularly preferred among such vectors are those inducible by environmental factors that are easy to manipulate, such as temperature and nutrient additives.

Expression vectors useful in the present invention include chromosomal-, episomal- and virus-derived vectors, e.g., vectors derived from bacterial plasmids or bacteriophages, and vectors derived from combinations thereof, such as cosmids and phagemids, and will preferably include at least one selectable marker such as a tetracycline or ampicillin resistance gene for culturing in a bacterial host cell. Prior to insertion into such an expression vector, the cDNA or amplified nucleic acid molecules of the invention should be operatively linked to an appropriate promoter, such as the phage lambda PL promoter, the E coli lac, trp and tac promoters. Other suitable promoters will be known to the skilled artisan.

Among vectors preferred for use in the present invention include pQE70, pQE60 and pQE-9, available from Qiagen; pBS vectors, Phagescript vectors, Bluescript vectors, pNH8A, pNHI6a, pNH18A, pNH46A, available from Stratagene; pcDNA3 available from Invitrogen; pGEX, pTrxfus, pTrc99a, pET-5, pET-9, pKK223-3, pKK233-3, pDR540, pRIT5 available from Pharmacia; and pSPORT1, pSPORT2 and pSV.multidot.SPORTI, available from Life Technologies, Inc. Other suitable vectors will be readily apparent to the skilled artisan.

The invention also provides methods of producing a recombinant host cell comprising the cDNA molecules, amplified nucleic acid molecules or recombinant vectors of the invention, as well as host cells produced by such methods. Representative host cells (prokaryotic or eukaryotic) that may be produced according to the invention include, but are not limited to, bacterial cells, yeast cells, plant cells and animal cells. Preferred bacterial host cells include Escherichia coli cells (most particularly E. coli strains DH10B and Stb12, which are available commercially (Life Technologies, Inc; Rockville, Md.)), Bacillus subtilis cells, Bacillus megaterium cells, Streptomyces spp. cells, Erwinia spp. cells, Klebsiella spp. cells and Salmonella typhimurium cells. Preferred animal host cells include insect cells (most particularly Spodoptera frugiperda SJ9 and Sf21 cells and Trichoplusa High-Five cells) and mammalian cells (most particularly CHO, COS, VERO, BHK and human cells). Such host cells may be prepared by well-known transformation, electroporation or transfection techniques that will be familiar to one of ordinary skill in the art.

In addition, the invention provides methods for producing a recombinant polypeptide, and polypeptides produced by these methods. According to this aspect of the invention, a recombinant polypeptide may be produced by culturing any of the above recombinant host cells under conditions favoring production of a polypeptide therefrom, and isolation of the polypeptide. Methods for culturing recombinant host cells, and for production and isolation of polypeptides therefrom, are well-known to one of ordinary skill in the art.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

Kits

The present compositions may be assembled into kits for use in reverse transcription or amplification of a nucleic acid molecule, or into kits for use in sequencing of a nucleic acid molecule. Kits according to this aspect of the invention comprise a carrier means, such as a box, carton, tube or the like, having in close confinement therein one or more container means, such as vials, tubes, ampules, bottles and the like. The first enzyme exhibiting a reverse transcriptase activity and the second enzyme exhibiting a 3′-5′ exonuclease activity may be in a single container as mixtures of the two enzymes, or in separate containers. The kits of the invention may also comprise (in the same or separate containers) one or more reverse transcriptases or DNA polymerases, a suitable buffer, one or more nucleotides and/or one or more primers or any other reagents described for compositions of the present invention.

The ratio of the first enzyme to the second enzyme in the subject kit may vary according to the present invention. Preferably, for a 20 μl reaction, the kit results in a working amount of 0.1-500 units of reverse transcriptase activity from the first enzyme, more preferably, 5-100 units of reverse transcriptase activity from the first enzyme, more preferably 10-50 units of reverse transcriptase activity from the first enzyme. Preferably, for a 20 μl reaction, the kit results in a working amount of 0.001-50 units of 3′-5′ exonuclease activity from the second enzyme, more preferably, 0.01-25 units of 3′-5′ exonuclease activity from the second enzyme, more preferably, 0.01-10 units of 3′-5′ exonuclease activity from the second enzyme. The ratio of the reverse transcriptase activity (in units) over the 3′-5′ exonuclease activity (in units) ranges from 5000 to 1, preferably, between 1500-5, more preferably between 100-10.

The kit of the present invention may include reagents facilitating the subsequent manipulation of cDNA synthesized as known in the art.

### EXAMPLES

The following examples further illustrate the present invention in detail but are not to be construed to limit the scope thereof.

### Example 1

### RT-PCR Reactions

The effect of addition of an enzyme having a 3′-5′ exonuclease activity during cDNA synthesis is examined using a blend containing E. coli epsilon subunit and RNase H⁻ M-MLV RT (StrataScript RT, Stratagene, Inc. CA) or AMV-RT.

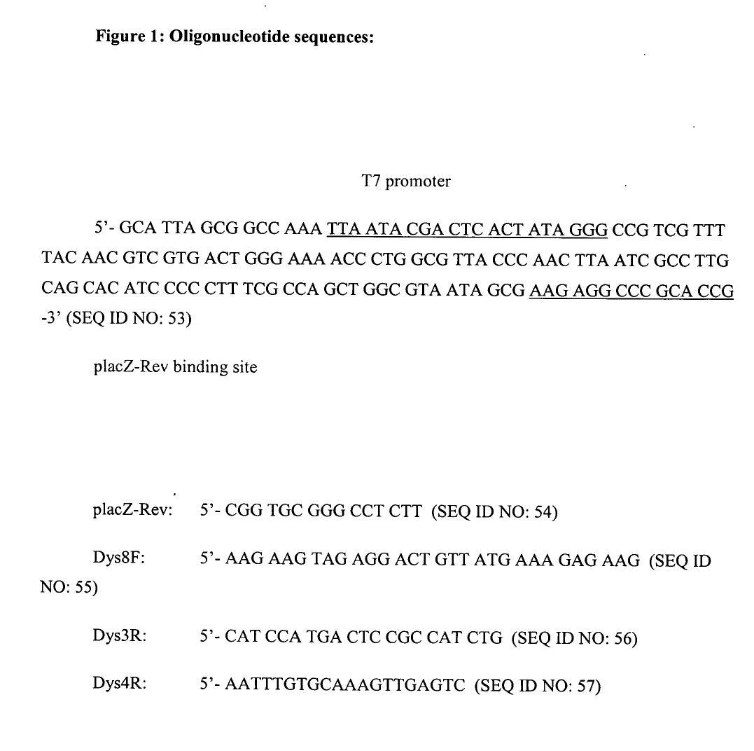

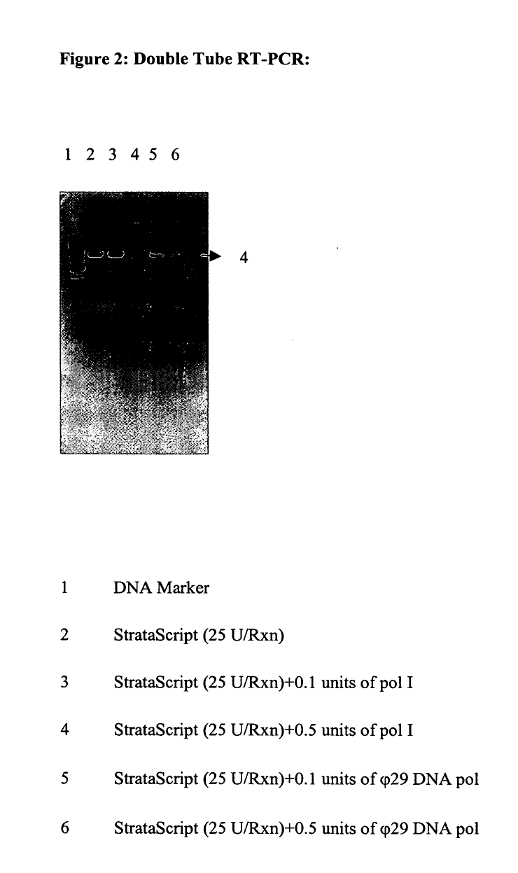

Each RT reaction was carried out in a total volume of 20 μl. The final reagent concentrations in each reaction were as follows: 500 ng human skeletal muscle total RNA, 500 ng oligo(dT)₁₈, and 4 mM total dNTPs in 1 x StrataScript buffer (Stratagene) for StrataScript or 100 mM Tris PH 8.3, 50 mM KCL, 10 mM MgCl₂, and 10 mM DTT for AMV-RT (RNase H⁺, Stratagene). All reactions were incubated at 42° C. for 60 minutes. 2 μl of each CDNA synthesis reaction was used in a PCR containing 2.5 units of TaqPlusPrecision (Stratagene), 1× TaqPlusPrecision buffer (Stratagene), 200 μM each dNTP, and 100 ng of each of the Dys8F (5′-AAG AAG TAG AGG ACT GTT ATG AAA GAG AAG) (SEQ ID NO:55) and Dys3R primers (5′-CAT CCA TGA CTC CGC CAT CTG) (SEQ ID NO:56) for amplification of the 4 kb fragment or the Dys8F and Dys4R 5′-AATTTGTGCAAAGTTGAGTC) (SEQ ID NO:57) primers for the amplification of the 6 kb fragment (FIG. 1). Amplification of the 4 kb fragment was carried out using the temperature cycling profile as follows: one cycle of 95° C. for 2 min, followed by 40 cycles of 95° C. for 30s, 55° C. for 30s, and 72° C. for 4 min using a PE9600 (Applied Biosystems). Amplification reactions for the 6 kb fragment were carried out using the temperature cycling profile as follows: one cycle of 95° C. for 2 min, followed by 40 cycles of 95° C. for 1 min, 55° C for 1 min, and 68° C. for 12min using a Robocycler (Stratagene). All PCR amplifications were performed with TaqPlus Precision (Stratagene). 8 μl of each reaction was run on a 1% agarose gel and stained with ethidium bromide (FIGS. 2-4).

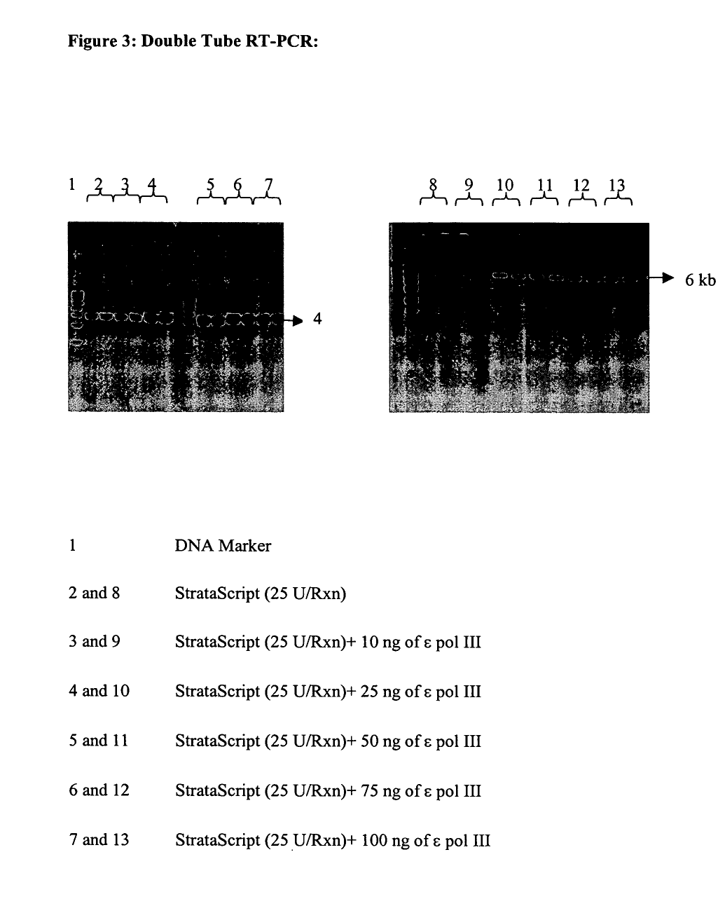

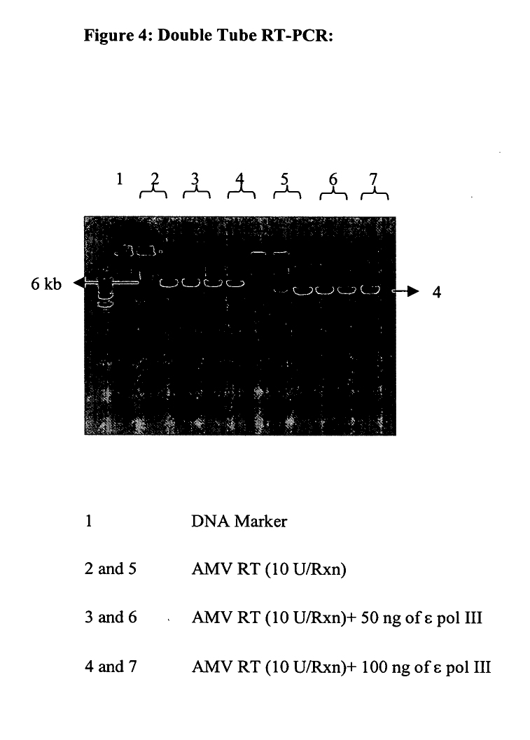

FIG. 2 shows that very low concentrations of E. coli DNA pol I and (φ29 DNA polymerase are inhibiting the RT reaction. FIG. 3, on the other hand, demonstrates that adding the ε subunit of E. coli DNA pol III to the RT reaction, increases the yield and length of cDNA amplified fragments significantly. The addition of the ε subunit of E. coli DNA pol III to the AMV-RT reaction demonstrates that the ε subunit also significantly increases the yield and length of cDNA synthesis by AMV-RT (FIG. 4). Therefore, enhancement by ε subunit is not limited to MMLV-RT, but appears to apply to broad class of RTs, including both monomeric (MMLV-like) and heterodimeric (AMV-like) RTs.

### Example 2

### Fidelity Assay

An 111 nucleotide fragment of the lacZα gene was fused to the T7 promoter (FIG. 1 ). The lacZα RNA for first strand DNA synthesis was produced by T7 RNA polymerase in vitro using the RNAMaxx Transcription kit (Stratagene Inc., CA) according to the manufacturer's recommendations. The purified RNA was dissolved in RNase free water.

cDNA synthesis: 500 ng of placZ-Rev was annealed to 2 μg of lacZ RNA by incubation at 60° C. for 3 minutes followed by 10 minutes cooling at room temperature. The extension reactions in 20 μl (in triplicates) contained 2 x StrataScript buffer, 25 units of StrataScript, 4 mM total dNTPs. For the fidelity assay, 25 units of StrataScript was used either alone or in combination with 50 ng of the ε subunit of Escherichia coli DNA polymerase III, 100 ng of p53 protein, 0.2 units of φ29 DNA polymerase, 0.1 units of Escherichia coli DNA polymerase I (non-inhibitory amount). The reactions were incubated at 42° C. for 60 minutes. The RNA was then hydrolyzed by the addition of 2 μl of RNace-IT (Stratagene, RNase-T1 5 U/μl, RNase A 2 μg/μl) and incubation at 37° C. for 30 minutes followed by 10 minutes at 80° C. The cDNA was then purified using RNA binding spin columns (Stratagene). 10-20% of the final cDNA product was used in a QuikChange reaction (Stratagene) to replace the wild type lacZ fragment. A 25 μl QuikChange reaction contained 2.5 μl 10× QuikChange Multi buffer (Stratagene), 15 units Taq DNA ligase (New England Biolabs), 50 ng of pBlueScript II (Stratagene), 0.8 mM total dNTPs, 2.5 units of PfuTurbo DNA polymerase. A 30 cycle PCR included 95° C. for 1 min, 55° C. for 1 min, and 65° C. for 6 minutes. The product was then digested with 10 units of DpnI at 37° C. for 60 minutes. 3 μl of this reaction was transformed into library efficiency DH5a competent cells (Invitrogen) and the cells were incubated at 37° C. overnight. The number of white colonies were then determined and divided by the total number of colonies to result in mutation frequency. Background mutation frequency was determined by direct sequencing of white colonies.

Results: After determining the average mutation frequencies from triplicate experiments in Tables II and III and subtracting the background from them, the fold difference in fidelity between StrataScript (RNase H minus MMLV-RT) and the blends are as follows:
1—RNase H minus MMLV-RT (StrataScript) plus the c subunit of Escherichia coli DNA polymerase III blend has 3 fold higher fidelity than RNase H minus MMLV-RT alone.

2—RNase H minus MMLV-RT plus Escherichia coli DNA pol I blend has 4 fold higher fidelity than RNase H minus MMLV-RT alone.

3—RNase H minus MMLV-RT plus φ29 DNA polymerase blend has 2.5 fold higher fidelity than RNase H minus MMLV-RT alone.

4—RNase H minus MMLV-RT plus p53 blend has 4 fold higher fidelity than RNase H minus MMLV-RT alone.

TABLE II
Mutation frequency comparisons (white colonies/total colony number): All
numbers include a background mutation frequency of 12.9 × 10⁻⁴

| | | S.S. (25 U/Rxn) + | S.S. (25 U/Rxn) + |
|---|---|---|---|
| | S.S. (25 U/Rxn) + | E. coli DNA pol I | φ29 DNA pol |
| StrataS. (25 U/Rxn) | p53 (100 ng/Rxn) | (0.1 U/Rxn) | (0.2 U/Rxn) |
| 79/25056 = 31.5 × 10⁻⁴ | 62/30306 = 20.4 × 10⁻⁴ | 63/34333 = 18.3 × 10⁻⁴ | 50/22746 = 22 × 10⁻⁴ |
| 98/30720 = 32 × 10⁻⁴ | 52/25491 = 20.4 × 10⁻⁴ | 58/36091 = 16.1 × 10⁻⁴ | 41/25760 = 16 × 10⁻⁴ |
| 82/28017 = 29.2 × 10⁻⁴ | 61/30128 = 20.2 × 10⁻⁴ | 24/13240 = 18.2 × 10⁻⁴ | 31/13990 = 22.1 × 10⁻⁴ |

TABLE III
Mutation frequency comparisons* (white
colonies/total colony number):

| | S.S. (25 U/Rxn) + ε subunit of DNA |
|---|---|
| StrataS. (25 U/Rxn) | pol III (50 ng/Rxn) |
| 117/34320 = 34.1 × 10⁻⁴ | 71/31984 = 22.2 × 10⁻⁴ |
| 182/49744 = 36.6 × 10⁻⁴ | 34/18174 = 18.7 × 10⁻⁴ |
| 81/23340 = 34.7 × 10⁻⁴ | 23/10698 = 21.5 × 10⁻⁴ |

*The background mutation frequency for the StrataScript reaction is 15.85 × 10⁻⁴ and for the blend with the ε subunit is 14.56 × 10⁻⁴.

### Other Embodiments

The foregoing examples demonstrate experiments performed and contemplated by the present inventors in making and carrying out the invention. It is believed that these examples include a disclosure of techniques which serve to both apprise the art of the practice of the invention and to demonstrate its usefulness. It will be appreciated by those of skill in the art that the techniques and embodiments disclosed herein are preferred embodiments only that in general numerous equivalent methods and techniques may be employed to achieve the same result.

All of the references identified hereinabove, are hereby expressly incorporated herein by reference to the extent that they describe, set forth, provide a basis for or enable compositions and/or methods which may be important to the practice of one or more embodiments of the present inventions.

1. A composition comprising a first enzyme exhibiting a reverse transcriptase activity and a second enzyme exhibiting a 3′-5′ exonuclease activity, wherein said second enzyme exhibiting a 3′-5′ exonuclease activity comprises an epsilon subunit from an eubacteria.

2. The composition of claim 1, wherein said second enzyme is thermostable.

3. The composition of claim 1, wherein said second enzyme is thermolabile.

4. The composition of claim 1, wherein said epsilon subunit is from E. coli.

5. The composition of claim 1, wherein said first enzyme exhibiting a reverse transcriptase activity is a DNA polymerase.

6. The composition of claim 5, wherein said DNA polymerase is a mutant DNA polymerase with an increased reverse transcriptase activity.

7. The composition of claim 1, wherein said first enzyme exhibiting a reverse transcriptase activity is a reverse transcriptase (RT).

8. The composition of claim 7, wherein said reverse transcriptase (RT) is a virus reverse transcriptase selected from the group consisting of: Moloney Murine Leukemia Virus (M-MLV) RT, Human Immunodeficiency Virus (HIV) RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus UR2AV RT, Avian Sarcoma Virus Y73 Helper Virus YAV RT, Rous Associated Virus (RAV) RT, and Myeloblastosis Associated Virus (MAV) RT.

9. The composition of claim 7, wherein said reverse transcriptase is M-MLV reverse transcriptase or AMV reverse transcriptase.

10. The composition of claim 7, wherein said reverse transcriptase is a reverse transcriptase with reduced RNase H activity.

11. The composition of claim 10, wherein said reverse transcriptase with reduced RNase H activity is an M-MLV reverse transcriptase with reduced RNase H activity or an AMV reverse transcriptase with reduced RNase H activity.

12. The composition of claim 1, wherein said first enzyme comprises an M-MLV reverse transcriptase with reduced RNase H activity and said second enzyme comprises E. coli DNA polymerase III epsilon subunit.

13. The composition of claim 12, wherein said M-MLV reverse transcriptase with reduced RNase H activity is added at a working amount of 0.1-500 units per 20 μl reaction.

14. The composition of claim 13, wherein said M-MLV reverse transcriptase with reduced RNase H activity is added at a working amount of 10-50 units per 20 μl reaction.

15. The composition of claim 14, wherein said M-MLV reverse transcriptase with reduced RNase H activity is added at a working amount of 20-40 units per 20 μl reaction.

16. The composition of claim 12, wherein said E. coli DNA polymerase III epsilon subunit is added at a working amount of 0.001-50 units per 20 μl reaction.

17. The composition of claim 16, wherein said E. coli DNA polymerase III epsilon subunit is added at a working amount of 0.01-25 units units per 20 μl reaction.

18. The composition of claim 17, wherein said E. coli DNA polymerase III epsilon subunit is added at a working amount of 0.01-10 units per 20 μl reaction.

19. A kit for cDNA synthesis comprising a first enzyme exhibiting a reverse transcriptase activity with reduced RNase H activity and a second enzyme exhibiting a 3′-5′ exonuclease activity and packaging materials therefor, wherein said second enzyme exhibiting a 3′-5′ exonuclease activity comprises an epsilon subunit from an eubacteria.

20. The kit of claim 19, wherein said epsilon subunit is from E. coli.

21. The kit of claim 19, wherein said first enzyme comprises M-MLV reverse transcriptase with reduced RNase H activity and said second enzyme comprises E. coli DNA polymerase III epsilon subunit.

22. The kit of claim 21, wherein said M-MLV reverse transcriptase with reduced RNase H activity is added at a working amount of 20-40 units per 20 μl reaction.

23. The kit of claim 21, wherein said E. coli DNA polymerase III epsilon subunit is added at a working amount of 0.01-10 units per 20 μl reaction.

24. The kit of claim 19, further comprising one or more of components selected from the group consisting of: one or more oligonucleotide primers, one or more nucleotides, a suitable buffer, one or more PCR accessory factors, and one or more terminating agents.

25. A method for cDNA synthesis comprising:
(a) contacting one or more nucleic acid templates with an enzyme composition comprising a first enzyme exhibiting a reverse transcriptase activity with reduced RNase H activity and a second enzyme exhibiting a 3′-5′ exonuclease activity, wherein said second enzyme exhibiting a 3′-5′ exonuclease activity comprises an epsilon subunit from an eubacteria; and
(b) incubating said templates and said enzyme composition under conditions sufficient to permit cDNA synthesis.

26. The method of claim 25, further comprising (c) incubating said synthesized cDNA under conditions sufficient to make one or more nucleic acid molecules complementary to said cDNA.

27. A method for amplifying one or more nucleic acid molecules, said method comprising:
(a) contacting one or more nucleic acid templates with an enzyme composition comprising a first enzyme exhibiting a reverse transcriptase activity with reduced RNase H activity and a second enzyme exhibiting a 3′-5′ exonuclease activity, wherein said second enzyme exhibiting a 3′-5′ exonuclease activity comprises an epsilon subunit from an eubacteria; and
(b) incubating said templates and said enzyme composition under conditions sufficient to permit amplification of one or more nucleic acid molecules.

28. The method of claim 25 or 27, wherein said nucleic acid template is a messenger RNA molecule or a population of MRNA molecules.

29. The method of claim 25 or 27, wherein said epsilon subunit is from E. coli.
